(19) United States
(12) Patent Application Publication
(10) Pub. No.: US 2021/0130493 A1
(43) Pub. Date: May 6, 2021

(54) MEDICAMENT COMPRISING ANTI-PHOSPHOLIPASE D4 ANTIBODY

(71) Applicant: SBI Biotech Co., Ltd., Tokyo (JP)

(72) Inventors: Tomohide Yamazaki, Tokyo (JP); Mayuki Endo, Tokyo (JP); Koji Ishida, Tokyo (JP)

(21) Appl. No.: 17/127,202

(22) Filed: Dec. 18, 2020

Related U.S. Application Data
(62) Division of application No. 14/908,004, filed on Jan. 27, 2016, now abandoned.

(30) Foreign Application Priority Data
Jul. 30, 2013 (JP) 2013-158258

(51) Int. Cl. C07K 16/40; G01N 33/573

(52) U.S. Cl. CPC C07K 16/40; G01N 33/573; A61K 2039/505; C07K 2317/73; C07K 2317/76; C07K 2317/24

(57) ABSTRACT

The present application provides the medicaments comprising the antibodies binding to phospholipase D4 (PLD4) as well as a method using said medicaments for detecting and suppressing activated B cells. The present application is further directed to therapy of auto-immune diseases and allergosis, resulting from the active-repressing function. In order to solves these problems, the present application provides that a monoclonal antibody binding to the extracellular domain of phospholipase D4 (PLD4) protein, or a fragment containing an antigen-binding region thereof as an active ingredient.

### TECHNICAL FIELD

The present invention relates to a use of an antibody binding to phospholipase D4. Hereinafter, “phospholipase D” may be abbreviated as PLD and “phospholipase D4” and the like may be abbreviated as PLD4 and the like.

### BACKGROUND ART

PLD is an enzyme which catalyzes a reaction to produce phosphatidic acid and choline by hydrolyzing phosphatidyl choline and causes various intracellular signaling. It has been believed that the produced phosphatidic acid functions as a lipid signal molecule.

PLD1 and PLD2 have been known as two types of mammal PLD, which have been previously known, and contain a phosphatidyl inositide-binding Phox homology domain (PX domain) and a phosphatidyl inositide-binding pleckstrin homology domain (PH domain) in the N-terminal region thereof. Both domains are involved in membrane localization of PLD.

PLD1 and PLD2 further contain two His-x-Lys-x-x-x-x-Asp sequences (HKD motifs). The HKD motifs are essential domains for PLD activity.

Phosphatidic acid produced by PLD1 and PLD2 has been suggested to be involved in cytoskeleton reconstruction, exocytosis, phagocytosis, canceration, cell adhesion, chemotaxis and the like, and mainly acts on nervous systems, immune systems and the like.

Human Hu-K4 and mouse SAM9, which are now officially named PLD3, lack the PX and PH domains and do not show PLD activity despite having two HKD motifs. Although there are further three PLD family members, PLD4, PLD5 and PLD6, little has been known about these non-classical PLDs.

As a result of searching a gene expression pattern in mouse cerebellar development in Cerebellar Development Transcriptome Database (CDT-DB), a transcription product, PLD4, controlled during the development was identified (see Non Patent Literature 1). Basic characteristics of PLD4 have not been reported. Enzymatic activity of PLD4 with or without glycosylation needs to be determined.

PLD4 has a 506 amino acid sequence shown in SEQ ID NO: 1 and is encoded by a cDNA base sequence of SEQ ID NO: 44 (Non Patent Literatures 1 and 2). The PLD4 protein has two tentative PDE regions (phosphodiesterase motifs) constituted of two HKD motifs (His-x-Lys-x-x-x-x-Asp amino acid sequence, x represents other amino acids) conserved in the C-terminal region, and a putative phosphorylation site (Thr 472). The structure of the PLD4 protein is estimated as a type II transmembrane protein. In addition, PLD4 does not have PX and PH domains, which PLD1 and PLD2 in a classical PLD family have them, in the N-terminal region.

On the other hand, PLD4 belongs to the PLD family because of having two HKD motifs, but lacks the PX domain and the PH domain and has a putative transmembrane domain instead (Non Patent Literature 3).

The expression of PLD4 mRNA has been found at low to medium levels in small cell clusters preferentially localized around white matter regions including corpus callosum and cerebellar white matter of 1 week old mice. These PLD4 mRNA-expressing cells have been identified as Iba1-positive microglia (Non Patent Literature 3). However, the PLD4-positive cells in mouse cerebellum is dispersed 10-day-old mice. It suggested that PLD4 expression is temporarily restricted during early postnatal development in mouse cerebellum.

Myelin formation in mouse begins in the corpora callosa and the cerebellar white matter at one week after birth. At this time, PLD4 is highly expressed in amoeboid (an activated state) microglia existing in the white matter, and thus it has been also believed that there is a possibility that PLD4-expressing cells in the white matter in this time are involved in myelin formation. In particular, it has also been revealed that PLD4 accumulates in food vacuoles, and it has been suggested that there is a possibility that PLD4 is involved in phagocytosis. In amoeboid microglia which is in an activated state, various cytokines and growth factors are secreted and simultaneously phagocytosis is activated. It has been believed that in the brain white matter of mouse in a developmental period, surplus oligodendrocytes (central nervous system glial cells, which form myelin by wrapping around axons) undergo apoptosis. There is a possibility that the oligodendrocytes are decomposed and removed in amoeboid microglia to secrete signal molecules and thereby adjust a myelin-forming environment in the white matter. It has been suggested that PLD4 is involved in these processes including the myelin formation.

Expression of mouse PLD4 mRNA is also observed in non-neuronal tissues and mainly distributed in the spleen. Strong expression of PLD4 protein is detected around a marginal zone of the splenic red pulp, and splenic PLD4 protein collected from subcellular membrane fractions is highly N-glycosylated. When PLD4 was expressed in a heterologous cell system, PLD4 was localized in the endoplasmic reticulum and Golgi apparatus. The heterologously expressed PLD4 did not show PLD enzyme activity (Non Patent Literature 3).

From the expression pattern of PLD4, which is spatiotemporally restricted, it has been suggested that PLD4 may play a role in common functions among the microglia and splenic marginal zone cells during early postnatal brain development.

On the other hand, the present inventors have found that PLD4 is specifically highly expressed in pDC (plasmacytoid Dendritic Cell) in a resting period (resting pDC) (Patent Literature 1). The present inventors further have reported that a PLD4-specific antibody can be utilized for suppression of pDC activity.

Further, PLD4 has been reported as one of novel susceptibility genes of Systemic Sclerosis in Japanese (Non Patent Literature 4). As a result of the same analysis in Europe, however, significant correlation with PLD4 has not been found and strong results showing a relationship between PLD4 and autoimmune diseases such as Systemic Sclerosis have not been obtained.

An immune mechanism is roughly classified into two groups. One is “natural immunity (innate immunity)” which detects foreign substances such as pathogens and carries out an initial attack, and the other is “acquired immunity” through information exchange which is presentation of antigen peptides and the like derived from foreign substances. Neutrophils, macrophage, dendritic cells (DC), NK (Natural Killer) cells and the like are mainly involved in the “natural immunity”, and T cells and B cells to which information of antigen peptides and the like presented by the above dendritic cells and the like is transmitted are involved in the “acquired immunity”. T cells activated by transmission of information of antigen peptides are capable of specifically recognizing and attacking pathogens in a direct manner as the cell-mediated immunity, and B cells activated in the same manner as above are capable of specific recognition and attack against pathogens in an indirect manner by producing antibodies (hormonal immunity).

In the “natural immunity”, pathogen-associated molecular patterns (PAMPs) universally existing in pathogens (LPS, CpG DNA, lipoproteins, RNA etc.) are recognized through Toll-like receptors (TLR), and secretion of inflammatory cytokines is promoted via NF-kB, or secretion of interferon (IFN) is promoted via IRF (Interferon regulatory factor). TLR is roughly classified into two groups by subcellular localization sites: a group expressed on cell surfaces and a group expressed in endosomes and endoplasmic reticula (ER). In pDC, IRF7 is activated via TLR7 and TLR9 localized in endosomes and endoplasmic reticula to induce IFN-α production. The reason why these TLRs are not expressed on cell surfaces but in cells has been suggested to decrease a risk of onset of autoimmune diseases. TLR7 and TLR9 recognize single-stranded RNA and DNA respectively as a ligand. Not only foreign pathogenic bacteria but also hosts hold these nucleic acids, and thus it has been suggested that receptors, which recognize nucleic acids and activate immune cells, always induce the autoimmune diseases.

On the other hand, B cells (B lymphocytes) showing an important role in the “acquired immunity” are lymphocytes which express immunoglobulin Ig receptors on the surface thereof. B cells are produced from hematopoietic stem cells in the bone marrow, and are differentiated into pre-B cells and immature B cells, and then mature into naive B cells (mature, unprimed B cells). The naive B cells are activated by not only the stimulation through the above T cells but also the direct antigen stimulation, and further become antibody-producing cells by differentiation and proliferation to produce and secrete antibodies such as IgM, IgD, IgA, IgE, IgG (including subclasses such as IgG1, IgG2, IgG2b, IgG3 and the like). It has been known that in addition to B cell receptors (BCR) recognizing specific foreign antigens, the above TLRs are expressed in B cells. It has been previously known, for example, that LPS which has been known to cause the proliferation and antibody production of B cells is a ligand of TLR4 and the above TLR7 and TLR9 are also expressed in B cells. Such B cells have been suggested to have a possibility to induce not only the above autoimmune diseases but also allergic diseases due to the overreaction of the antibody-producing ability thereof.

IgG, immunoglobulin G, is an antibody isotype consisting of four peptide chains—two identical heavy chains and two identical light chains. IgG is produced by B cells and plays a critical role for adaptive immunity. Naive B cells which do not produce IgG, differentiate into plasmablasts, and eventually into plasma cells. Plasmablasts and plasma cells can produce a large amount of antibodies. Conventionally, myeloid dendritic cells (DCs) have been shown to trigger B cell growth and differentiation by stimulating with IL-12 and IL-6 and/or membrane molecules such as BAFF/APRIL (Non Patent Literatures 5, 6 and 7). In addition, plasmacytoid DCs (pDCs) induce maturation and differentiation of naive B cells into antibody-secreting plasmablasts and plasma cells producing IFN-α and IL-6 (Non Patent Literature 8). The variable region of IgG captures various pathogens such as viruses, bacteria, and fungi, resulting in protection of the body from such infections.

SLE is regarded as a classic immune complex-mediated autoimmune disease. Immune complexes (ICs) are formed in circulation or in situ as a result of produced auto-antibodies against nucleic acids and their associated proteins, such as dsDNA, ribonucleoprotein, and histone. Such ICs cause inflammation with disease-characteristic clinical symptoms such as nephritis, arthritis, skin rashes, and vasculitis. Blood from SLE patient is characterized by reduction of naive B cells and increased memory B cells, plasmablasts and plasma cells (Non Patent Literatures 9, 10 and 11). Therefore, suppression of differentiation into plasma cells and antibody production through manipulation of auto-reactive antibody-secreting plasmablasts would result in a promising strategy to cure autoimmune diseases.

In PBMCs, there are various subsets of B cells, such as naive B cells, memory B cells, and plasmablasts. Most of B cell subset in PBMCs is naive B cells. Naive B cells are the one who are not exposed by foreign antigen. Memory B cells are the one who are formed by primary infection and are critical in quick antibody-mediated immune response by differentiation into plasmablasts. Plasmablasts are the one who secrete a large amount of antibody and marked by CD19+CD27+IgD−CD38+.

Once exposed by foreign antigen, naive B cells become activated B cells. The activated B cells are further differentiated in to memory B cell and/or also plasmablasts that secrete antibodies. This change is called “maturation”.

B cell maturation occurs in multiple phases. The initial, antigen-independent phase induces mature B cells that can bind to a unique antigen. This stage of maturation happens in the bone marrow and the spleen in living body. The antigen-dependent phase of B cell maturation happens following B cell activation by antigen binding and co-stimulation. These signals promote B cell maturation into either memory B cells or antibody-secreting plasmablasts. The antigen-dependent phase of B cell maturation involves activated B cell proliferation, antibody affinity maturation, and antibody class switching. Those maturations occur in the germinal centers of secondary lymphoid tissues.

It has been reported that, in vitro experimental condition, pDCs induce the maturation of activated B cells into Ig-secreting plasmablasts through release of IFN-α and IL-6. CpG2216 activates pDCs to induce IFN-α production and B cells to initiate maturation. IFN-α from pDCs further supports maturation of activated B cells into plasmablasts in the presence of IL-6.

### CITATION LIST

### Patent Literature

[PTL 1] PCT/JP2013/052781

### Non Patent Literature

[NPL 1] Tao et al., Nat. Methods 2(8), pp 591-598 (2005)
[NPL 2] Clark et al., Genome Res. 13(10), pp 2265-2270 (2003)
[NPL 3] Plos ONE www.plosone.org, November 2010, Volume 5, Issue 11, e13932
[NPL 4] ARTHRITIS & RHEUMATISM Vol. 65, No. 2, February 2013, pp 472-480
[NPL 5] Balazs et al., 2002, Immunity, 17, 341-352
[NPL 6] Litinskiy et al., 2002, Nat Immunol, 3, 822-829
[NPL 7] MacLennan and Vinuesa, 2002, Immunity, 17, 235-238
[NPL 8] Jego et al, 2003, Immunity, 19, 225-234
[NPL 9] Odendahl et al., 2000, JI, 165, 5970-5979
[NPL 10] Arce et al., 2001, JI, 167, 2361-2369
[NPL 11] Wei et al., 2007, JI, 178, 6624-6633

### SUMMARY

### Technical Problem

A problem to be solved by the present invention is to regulate activated B cells using an antibody binding to PLD4 and to improve symptoms of diseases caused thereby.

### Solution to Problem

Through research on PLD4, the present inventors verified that in addition to pDC cells in a resting period which have been previously reported, PLD4 expression was also induced in activated B cells. The present inventors therefore examined influence of PLD4 antibodies on activated B cells. A method for producing and purifying anti-PLD4 antibodies is carried out by a method in Patent Literature 1.

That is, the present invention relates to a second use using anti-PLD4 antibodies described below.

(1) A pharmaceutical composition for suppressing activated B cells, wherein the pharmaceutical composition comprises a monoclonal antibody binding to a phospholipase D4 (PLD4) protein, or a fragment containing an antigen-binding region thereof as an active ingredient.
(2) The pharmaceutical composition according to (1) above, wherein the monoclonal antibody or the fragment containing the antigen-binding region thereof has a sequence SYWMH (SEQ ID NO: 2) as CDR1, a sequence DIYPGSDSTNYNEKFKS (SEQ ID NO: 3) as CDR2 and GGWLDAMDY (SEQ ID NO: 4) as a sequence CDR3 in a variable region of a heavy chain.
(3) The pharmaceutical composition according to (1) above, wherein the monoclonal antibody or the fragment containing the antigen-binding region thereof has a sequence RASQDISNYLN (SEQ ID NO: 5) as CDR1, a sequence YTSRLHS (SEQ ID NO: 6) as CDR2 and a sequence QQGNTLPW (SEQ ID NO: 7) as CDR3 in a variable region of a light chain.
(4) The pharmaceutical composition according to (1) above, wherein the monoclonal antibody or the fragment containing the antigen-binding region thereof has the sequence SYWMH as CDR1, the sequence DIYPGSDSTNYNEKFKS as CDR2 and the sequence GGWLDAMDY as CDR3 in the variable region of the heavy chain, and has the sequence RASQDISNYLN as CDR1, the sequence YTSRLH as CDR2 and the sequence QQGNTLPW as CDR3 in the variable region of the light chain.
(5) The pharmaceutical composition according to (1) above, wherein the monoclonal antibody or the fragment containing the antigen-binding region thereof has a sequence TYWMH (SEQ ID NO: 8) as CDR1, a sequence AIYPGNSETSYNQKFKG (SEQ ID NO: 9) as CDR2 and GYSDFDY (SEQ ID NO: 10) as a sequence CDR3 in the variable region of the heavy chain.
(6) The pharmaceutical composition according to (1) above, wherein the monoclonal antibody or the fragment containing the antigen-binding region thereof has a sequence HASQGIRSNIG (SEQ ID NO: 11) as CDR1, a sequence HGTNLED (SEQ ID NO: 12) as CDR2 and a sequence VQYVQFP (SEQ ID NO: 13) as CDR3 in the variable region of the light chain.
(7) The pharmaceutical composition according to (1) above, wherein the monoclonal antibody or the fragment containing the antigen-binding region thereof has the sequence TYWMH as CDR1, the sequence AIYPGNSETSYNQKFKG as CDR2 and the sequence GYSDFDY as CDR3 in the variable region of the heavy chain, and has the sequence HASQGIRSNIG as CDR1, the sequence HGTNLED as CDR2 and the sequence VQYVQFP as CDR3 in the variable region of the light chain.
(8) The pharmaceutical composition according to (1) above, wherein the monoclonal antibody or the fragment containing the antigen-binding region thereof has a sequence DYNLH (SEQ ID NO: 14) as CDR1, a sequence YIYPYNGNTGYNQKFKR (SEQ ID NO: 15) as CDR2 and GGIYDDYYDYAIDY (SEQ ID NO: 16) as a sequence CDR3 in the variable region of the heavy chain.
(9) The pharmaceutical composition according to (1) above, wherein the monoclonal antibody or the fragment containing the antigen-binding region thereof has a sequence RASENIYSHIA (SEQ ID NO: 17) as CDR1, a sequence GATNLAH (SEQ ID NO: 18) as CDR2 and a sequence QHFWGTP (SEQ ID NO: 19) as CDR3 in the variable region of the light chain.
(10) The pharmaceutical composition according to (1) above, wherein the monoclonal antibody or the fragment containing the antigen-binding region thereof has the sequence DYNLH as CDR1, the sequence YIYPYNGNTGYNQKFKR as CDR2 and the sequence GGIYDDYYDYAIDY as CDR3 in the variable region of the heavy chain, and has the sequence RASENIYSHIA as CDR1, the sequence GATNLAH as CDR2 and the sequence QHFWGTP as CDR3 in the variable region of the light chain.
(11) The pharmaceutical composition according to (1) above, wherein the monoclonal antibody or the fragment containing the antigen-binding region thereof has a sequence SYYLY (SEQ ID NO: 20) as CDR1, a sequence LINPTNSDTIFNEKFKS (SEQ ID NO: 21) as CDR2 and EGGYGYGPFAY (SEQ ID NO: 22) as a sequence CDR3 in the variable region of the heavy chain.
(12) The pharmaceutical composition according to (1) above, wherein the monoclonal antibody or the fragment containing the antigen-binding region thereof has a sequence TSSQTLVHSNGNTYLH (SEQ ID NO: 23) as CDR1, a sequence KVSNRFS (SEQ ID NO: 24) as CDR2 and a sequence HSTHVP (SEQ ID NO: 25) as CDR3 in the variable region of the light chain.
(13) The pharmaceutical composition according to (1) above, wherein the monoclonal antibody or the fragment containing the antigen-binding region thereof has the sequence SYYLY as CDR1, the sequence LINPTNSDTIFNEKFKS as CDR2 and the sequence EGGYGYGPFAY as CDR3 in the variable region of the heavy chain, and has the sequence TSSQTLVHSNGNTYLH as CDR1, the sequence KVSNRFS as CDR2 and the sequence HSTHVP as CDR3 in the variable region of the light chain.
(14) The pharmaceutical composition according to (1) above, wherein the monoclonal antibody or the fragment containing the antigen-binding region thereof has a sequence SYGMS (SEQ ID NO: 26) as CDR1, a sequence TISSGGSYIYYPESVKG (SEQ ID NO: 27) as CDR2 and LYGGRRGYGLDY (SEQ ID NO: 28) as a sequence CDR3 in the variable region of the heavy chain.
(15) The pharmaceutical composition according to (1) above, wherein the monoclonal antibody or the fragment containing the antigen-binding region thereof has a sequence RSSKSLLHSDGITYLY (SEQ ID NO: 29) as CDR1, a sequence QMSNLAS (SEQ ID NO: 30) as CDR2 and a sequence AQNLEL (SEQ ID NO: 31) as CDR3 in the variable region of the light chain.
(16) The pharmaceutical composition according to (1) above, wherein the monoclonal antibody or the fragment containing the antigen-binding region thereof has the sequence SYGMS as CDR1, the sequence TISSGGSYIYYPESVKG as CDR2 and the sequence LYGGRRGYGLDY as CDR3 in the variable region of the heavy chain, and has the sequence RSSKSLLHSDGITYLY as CDR1, the sequence QMSNLAS as CDR2 and the sequence AQNLEL as CDR3 in the variable region of the light chain.
(17) The pharmaceutical composition according to (1) above, wherein the monoclonal antibody or the fragment containing the antigen-binding region thereof has a sequence SHYYWT (SEQ ID NO: 32) as CDR1, a sequence YISYDGSNNYNPSLKN (SEQ ID NO: 33) as CDR2 and EGPLYYGNPYWYFDV (SEQ ID NO: 34) as a sequence CDR3 in the variable region of the heavy chain.
(18) The pharmaceutical composition according to (1) above, wherein the monoclonal antibody or the fragment containing the antigen-binding region thereof has a sequence RASQDIDNYLN (SEQ ID NO: 35) as CDR1, a sequence YTSRLHS (SEQ ID NO: 36) as CDR2 and a sequence QQFNTLP (SEQ ID NO: 37) as CDR3 in the variable region of the light chain.
(19) The pharmaceutical composition according to (1) above, wherein the monoclonal antibody or the fragment containing the antigen-binding region thereof has the sequence SHYYWT as CDR1, the sequence YISYDGSNNYNPSLKN as CDR2 and the sequence EGPLYYGNPYWYFDV as CDR3 in the variable region of the heavy chain, and has the sequence RASQDIDNYLN as CDR1, the sequence YTSRLHS as CDR2 and the sequence QQFNTLP as CDR3 in the variable region of the light chain.
(20) The pharmaceutical composition according to (1) above, wherein the monoclonal antibody or the fragment containing the antigen-binding region thereof has a sequence SHYYWS (SEQ ID NO: 38) as CDR1, a sequence YISYDGSNNYNPSLKN (SEQ ID NO: 39) as CDR2 and EGPLYYGNPYWYFDV (SEQ ID NO: 40) as a sequence CDR3 in the variable region of the heavy chain.
(21) The pharmaceutical composition according to (1) above, wherein the monoclonal antibody or the fragment containing the antigen-binding region thereof has a sequence RASQDIDNYLN (SEQ ID NO: 41) as CDR1, a sequence YTSRLHS (SEQ ID NO: 42) as CDR2 and a sequence QQFNTLP (SEQ ID NO: 43) as CDR3 in the variable region of the light chain.
(22) The pharmaceutical composition according to (1) above, wherein the monoclonal antibody or the fragment containing the antigen-binding region thereof has the sequence SHYYWS as CDR1, the sequence YISYDGSNNYNPSLKN as CDR2 and the sequence EGPLYYGNPYWYFDV as CDR3 in the variable region of the heavy chain, and has the sequence RASQDIDNYLN as CDR1, the sequence YTSRLHS as CDR2 and the sequence QQFNTLP as CDR3 in the variable region of the light chain.
(23) A pharmaceutical composition for suppressing activated B cells, wherein the pharmaceutical composition comprises a monoclonal antibody produced by any of hybridomas mp5B7, mp7B4, mp13D4 and mp13H11 of Deposit Nos. NITE BP-1211, NITE BP-1212, NITE BP-1213 and NITE BP-1214, or a fragment containing an antigen-binding region thereof as an active ingredient.
(24) The pharmaceutical composition according to any one of (1) to (23) above, further for preventing or treating autoimmune diseases.
(25) The pharmaceutical composition according to any one of (1) to (23) above, further for preventing or treating allergic diseases.
(26) A method for detecting activated B cells, the method including a step of bringing a monoclonal antibody binding to an extracellular domain of PLD4 or a fragment containing an antigen-binding region thereof into contact with cells to be tested and detecting the monoclonal antibody or the fragment containing the antigen-binding region thereof which binds to the cells.
(27) A reagent for detecting activated B cells, wherein the reagent comprises a monoclonal antibody binding to an extracellular domain of PLD4 or a fragment containing an antibody-binding region thereof.
(28) A method for suppressing activated B cells, the method including a step of bringing either of the following components into contact with activated B cells:

(a) a monoclonal antibody which binds to PLD4 and suppresses activated B cells, or a fragment containing an antigen-binding region thereof, and

(b) immunoglobulin into which a complementarity-determining region of the monoclonal antibody in (a) is grafted, or a fragment containing an antigen-binding region thereof.

(29) A method for suppressing activated B cells in a living body, the method including a step of administering either of the following components to the living body:

(a) a monoclonal antibody which binds to PLD4 and suppresses an activity of activated B cells, or a fragment containing an antigen-binding region thereof, and

(b) immunoglobulin into which a complementarity-determining region of the monoclonal antibody in (a) is grafted, or a fragment containing an antigen-binding region thereof.

(30) The method according to (28) above or (29) above, wherein the activity of the activated B cells is an antibody-producing activity.
(31) An agent for suppressing activated B cells, wherein the agent comprises either of the following components as an active component:

(a) a monoclonal antibody which binds to PLD4 and suppresses activated B cells, or a fragment containing an antigen-binding region thereof, and

(b) immunoglobulin into which a complementarity-determining region of the monoclonal antibody in (a) is grafted, or a fragment containing an antigen-binding region thereof.

(32) The agent for suppressing activated B cells according to (31) above, wherein an activity of the activated B cells is an antibody-producing activity.

The “activated B cells” may include B cells possessing the activity of proliferation and antibody production and secretion by not only direct stimulation through BCR and TLR but also stimulation through T cells.

The “fragment containing an antigen-binding region” may include Fab, Fab′, F(ab′)₂ fragments and the like obtained by partial digestion with papain or pepsin, but is not limited thereto. In addition, the fragment containing an antigen-binding region also may include a fragment of immunoglobulin containing a variable region into which CDR (complementarily-determining region) of a monoclonal antibody is grafted. It is well known that these antibody fragments can be used as antibody molecules having binding affinity to antigens. Alternatively, insofar as required antigen-binding activity is maintained, antibodies constructed by gene recombination can be used. Examples of antibodies constructed by gene recombination can include chimeric antibodies, CDR-grafted antibodies, single chain Fv (scFv), diabody (diabodies), linear antibodies, and polyspecific antibodies formed from antibody fragments and the like. A method for obtaining these antibodies based on monoclonal antibodies or antibody-producing cells producing the monoclonal antibodies is known.

The “autoimmune diseases” are diseases which are caused by attacks of immune functions by misunderstanding one's own body tissues as foreign substances. Organ-specific autoimmune diseases include Guillain-Barre syndrome, myasthenia gravis, chronic gastritis (chronic atrophic gastritis), autoimmune hepatitis, primary biliary cirrhosis, primary sclerosing cholangitis, autoimmune pancreatitis, aortitis syndrome, Goodpasture syndrome, rapidly progressive glomerulonephritis, megaloblastic anemia, autoimmune hemolytic anemia, autoimmune neutropenia, idiopathic thrombocytopenic purpura, Basedow disease, Hashimoto thyroiditis, primary hypothyroidism, idiopathic Addison's disease, type 1 diabetes, ulcerative colitis, Crohn's disease, celiac disease and the like; and systemic autoimmune diseases include articular rheumatism, systemic lupus erythematosus, anti-phospholipid antibody syndrome, polymyositis, scleroderma, Sjogren's syndrome, vasculitis syndrome, autoimmune lymphoproliferative syndrome (ALPS) and the like, but are not limited thereto.

The “allergic diseases” are diseases caused by abnormal immune reactions against foreign substances, and include atopic dermatitis, bronchial asthma, pollinosis, allergic rhinitis, urticaria, infantile asthma, allergic gastroenteritis, contact dermatitis, serum sickness, vascular purpura and the like but are not limited thereto.

### Advantageous Effects of Invention

The present invention provides a therapeutic method attributable to suppression of activated B cells using an antibody specifically recognizing PLD4 and a fragment thereof, and a medicament having its therapeutic effect.

The present invention can be further expected to have preventive and therapeutic effects on patients with autoimmune diseases or allergic diseases by using the activated B cell-suppressing activity.

### BRIEF DESCRIPTION OF DRAWINGS

FIG. 1 is a FACS analysis diagram which shows staining of human B cells (CD19+) with anti-PLD4 antibodies. PLD4 protein was induced on CD19+ B cells by stimulation with TLR9 ligand, CpG2006. Induction of PLD4 in activated B cells (CD19+) could be detected by a TLR9 ligand (CpG2006). Monoclonal antibodies 11G9.6 and 5B7 were used to detect PLD4. Mouse IgG2b, [symbol] was used as a negative control.

FIG. 2 is a FACS analysis diagram which shows staining of human PBMC with an anti-PLD4 antibody and an anti-CD19 antibody. PLD4+ cells were increased in activated B cells (CD19+) by stimulation with TLR9 ligand. Mouse IgG1, [symbol] was used as a negative control.

FIG. 3 is a FACS analysis diagram which shows staining of human PBMC with anti-PLD4 antibodies and an anti-CD19 antibody in the presence or absence of TLR9 ligand stimulation. A significant increase of PLD4+ TLR9 ligand-stimulated B cells (CD19+) could be detected with anti-PLD4 antibodies (5B7, 13D4, 13H11 and 11G9.6). Mouse IgG2b, [symbol] was used as a negative control.

FIG. 4 is a FACS analysis diagram which shows reduction of PLD4+ activated B cells by the indicated each anti-PLD4 chimeric antibody. Co-culture of PBMCs with the anti-PLD4 chimeric antibodies (ch3B4, ch13D4, ch13H11, ch5B7 and ch11G9.6) reduced PLD4+ activated B cells in the presence of TLR9 ligand. In a case in which an antibody was not added (NoAb) and a case in which a non-specific antibody was used (Control Ig), however, the activation of B cells by adding CpG2006 could not be suppressed.

FIG. 5 is a diagram in which suppressive effect in FIG. 4 is expressed in numbers. An activated B cell group which expresses PLD4 and was treated with control Ig is considered as 100% and changes in an activated B cell group which expresses PLD4 and was treated with each anti-PLD4 chimeric antibody are shown.

FIG. 6 is a result of flow cytometry. PBMCs were cultured with the indicated chimeric PLD4 antibodies in the presence of TLR9 ligand and recombinant human IL-6. Plasmablast population (CD19+CD27+IgD−CD38+) was reduced by the treatment with ch3B4, ch5B7, ch13D4, ch13H11, or ch11G9.6 compared with control Ig treatment.

FIG. 7 is a result of ELISA assay of the culture supernatant of FIG. 6. Human IgG production from plasmablasts was reduced by the treatment with ch3B4, ch5B7, ch13D4, ch13H11, or ch11G9.6 compared with control Ig treatment.

### DESCRIPTION OF EMBODIMENTS

The present inventors newly found that PLD4 was a molecule whose expression is induced with activation of B cells.

The present inventors have previously reported expression, subcellular localization, structure and function of human PLD4 (Patent Literature 1). In the present invention, it further turned out that the expression of PLD4 is induced in not only pDC but also activated B cells. It was further newly found that anti-PLD4 antibodies suppressed activated B cells. Such findings not only strengthen a possibility that anti-PLD4 antibodies have a therapeutic effect on autoimmune diseases by suppression of pDC activity, which has been previously reported, but also B cell activity.

Proteins such as CD19, CD20, CD22 and BAFF-R are expressed on the surface of B cells. CD19 is expressed on B cells from an early stage such as pro-B cells to antibody-secreting plasma cells, and functions as an auxiliary receptor controlling activation in mature B cells. CD20 is expressed from pre B cells to activated B cells, CD22 is expressed on the cell surface of mature B cells, and the expression of BAFF-R is observed in the extensive differentiation stage of B cells. Therefore, there is concern that antibodies recognizing these proteins suppress not only activated B cells but also unprimed naive B cells. The anti-PLD4 antibodies of the present invention are however characterized by suppressing activated B cells without influence on naive B cells.

The anti-PLD4 antibodies used in the present invention are the same as those reported previously (Patent Literature 1). In short, using as an immunogen a recombinant PLD4-Ig fusion protein encoding an amino acid sequence containing an extracellular domain of PLD4 (the amino acid sequence corresponding to from position 54 to 506 in the amino acid sequence shown in SEQ ID NO: 1), an antibody against PLD4 was obtained as follows.

### <Creation of Anti-Human PLD4 Monoclonal Antibodies>

### 1) Immunization

As an immunogen, the above recombinant PLD4-Ig fusion protein was used. The PLD4-Ig fusion protein was administered to the dorsal hypodermis of three BALB/c mice. As adjuvants, Freund's Adjuvants, Complete and Incomplete (SIGMA), were used. The volume of first administration was 200 μg/mouse, and the volume of second to fourth administration was 50 μg/mouse.

### 2) Confirmation of Anti-Serum Titer

Blood was collected after third and fourth immunization and anti-serum titer was evaluated by ELISA.

The PLD4-Ig fusion protein was transformed into a solid phase on a 96 well microtiter plate. An antiserum was serially diluted in 3-fold increments from 1000-fold and a dilution series up to 729000-fold was prepared. To the antigen-coated plate, each 50 μl of each sample was added and a first-order reaction was carried out. After washing, a second-order reaction was carried out with the HRP-labeled anti-mouse IgG ( [symbol] , λ) antibody and color development was detected with OPD (orthophenylene diamine) (490 nm).

### 3) Cell Fusion

Splenic cells were extracted from mice in which an increase in anti-serum titer was observed. The extracted splenic cells and mouse myeloma cells (P3U1) were fused by the PEG method and the fused splenic cells were selectively cultured in an HAT medium.

### <FACS Screening of Hybridomas Using CAL-1 Cells>

An antibody produced from each clone of the fused splenic cells obtained by HAT selective culture was evaluated by FACS. Consequently, 3B4, 5B7, 7B4, 8C11, 10C3, 11D10, 13D4, 13H11, 14C1 and 11G9.6 in hybridoma culture supernatant well reacted to human PLD4.

In each monoclonal antibody produced from the above hybridomas, CDR regions (CDRs; CDR1, CDR2 and CDR3) and FW regions (Frame work regions) in a variable region and a sequence of the variable region were determined according to an analytical method of Kabat numbering system (Kabat et al, 1991, Sequences of Proteins of Immunological Interest, National Institutes of Health Publication No. 91-3242, 5th ed., United States Department of Health and Human Services, Bethesda, Md.).

The nucleic acid sequence of the heavy chain variable region of the obtained mouse 11G9.6 antibody is SEQ ID NO: 74, and the amino acid sequence is SEQ ID NO: 75. The amino acid sequences of CDR1, CDR2 and CDR3 within the heavy chain variable region of the mouse 11G9.6 antibody are SEQ ID NO: 2, SEQ ID NO: 3 and SEQ ID NO: 4, respectively.

The nucleic acid sequence of the heavy chain variable region of the obtained mouse 3B4 antibody is SEQ ID NO: 76, and the amino acid sequence is SEQ ID NO: 77. The amino acid sequences of CDR1, CDR2 and CDR3 within the heavy chain variable region of the mouse 3B4 antibody are SEQ ID NO: 8, SEQ ID NO: 9 and SEQ ID NO: 10, respectively.

The nucleic acid sequence of the heavy chain variable region of the obtained mouse 5B7 antibody is SEQ ID NO: 78, and the amino acid sequence is SEQ ID NO: 79. The amino acid sequences of CDR1, CDR2 and CDR3 within the heavy chain variable region of the mouse 5B7 antibody are SEQ ID NO: 14, SEQ ID NO: 15 and SEQ ID NO: 16, respectively.

The nucleic acid sequence of the heavy chain variable region of the obtained mouse 7B4 antibody is SEQ ID NO: 80, and the amino acid sequence is SEQ ID NO: 81. The amino acid sequences of CDR1, CDR2 and CDR3 within the heavy chain variable region of the mouse 7B4 antibody are SEQ ID NO: 14, SEQ ID NO: 15 and SEQ ID NO: 16, respectively. The 7B4 antibody is an antibody which has the same CDR sequences in the variable regions of the heavy and light chains as of the 5B7 antibody.

The nucleic acid sequence of the heavy chain variable region of the obtained mouse 8C11 antibody is SEQ ID NO: 82, and the amino acid sequence is SEQ ID NO: 83. The amino acid sequences of CDR1, CDR2 and CDR3 within the heavy chain variable region of the mouse 8C11 antibody are SEQ ID NO: 20, SEQ ID NO: 21 and SEQ ID NO: 22, respectively.

The nucleic acid sequence of the heavy chain variable region of the obtained mouse 10C3 antibody is SEQ ID NO: 84, and the amino acid sequence is SEQ ID NO: 85. The amino acid sequences of CDR1, CDR2 and CDR3 within the heavy chain variable region of the mouse 10C3 antibody are SEQ ID NO: 26, SEQ ID NO: 27 and SEQ ID NO: 28, respectively.

The nucleic acid sequence of the heavy chain variable region of the obtained mouse 11D10 antibody is SEQ ID NO: 86, and the amino acid sequence is SEQ ID NO: 87. The amino acid sequences of CDR1, CDR2 and CDR3 within the heavy chain variable region of the mouse 11D10 antibody are SEQ ID NO: 26, SEQ ID NO: 27 and SEQ ID NO: 28, respectively. The 11D10 antibody is an antibody which has the same CDR sequences in the variable regions of the heavy and light chains as of the 10C3 antibody. Their heavy chain isotypes are, however, different (10C3 has the constant region of mouse IgG2a and 11D10 has the constant region of mouse IgG2b).

The nucleic acid sequence of the heavy chain variable region of the obtained mouse 13D4 antibody is SEQ ID NO: 88, and the amino acid sequence is SEQ ID NO: 89. The amino acid sequences of CDR1, CDR2 and CDR3 within the heavy chain variable region of the mouse 13D4 antibody are SEQ ID NO: 32, SEQ ID NO: 33 and SEQ ID NO: 34, respectively.

The nucleic acid sequence of the heavy chain variable region of the obtained mouse 13H11 antibody is SEQ ID NO: 90, and the amino acid sequence is SEQ ID NO: 91. The amino acid sequences of CDR1, CDR2 and CDR3 within the heavy chain variable region of the mouse 13H11 antibody are SEQ ID NO: 38, SEQ ID NO: 39 and SEQ ID NO: 40, respectively.

The nucleic acid sequence of the heavy chain variable region of the obtained mouse 14C1 antibody is SEQ ID NO: 92, and the amino acid sequence is SEQ ID NO: 93. The amino acid sequences of CDR1, CDR2 and CDR3 within the heavy chain variable region of the mouse 14C1 antibody are SEQ ID NO: 38, SEQ ID NO: 39 and SEQ ID NO: 40, respectively. The 14C1 antibody is an antibody which has the same CDR sequences in the variable regions of the heavy and light chains as of the 13H11 antibody. Their heavy chain isotypes are, however, different (13H11 has the constant region of mouse IgG2b and 14C1 has the constant region of mouse IgG1).

The nucleic acid sequence of the light chain variable region of the mouse 11G9.6 antibody is SEQ ID NO: 94, and the amino acid sequence is SEQ ID NO: 95. The amino acid sequences of CDR1, CDR2 and CDR3 within the light chain variable region of the mouse 11G9.6 antibody are SEQ ID NO: 5, SEQ ID NO: 6 and SEQ ID NO: 7, respectively.

The nucleic acid sequence of the light chain variable region of the mouse 3B4 antibody is SEQ ID NO: 96, and the amino acid sequence is SEQ ID NO: 97. The amino acid sequences of CDR1, CDR2 and CDR3 within the light chain variable region of the mouse 3B4 antibody are SEQ ID NO: 11, SEQ ID NO: 12 and SEQ ID NO: 13, respectively.

The nucleic acid sequence of the light chain variable region of the mouse 5B7 antibody is SEQ ID NO: 98, and the amino acid sequence is SEQ ID NO: 99. The amino acid sequences of CDR1, CDR2 and CDR3 within the light chain variable region of the mouse 5B7 antibody are SEQ ID NO: 17, SEQ ID NO: 18 and SEQ ID NO: 19, respectively.

The nucleic acid sequence of the light chain variable region of the mouse 7B4 antibody is SEQ ID NO: 100, and the amino acid sequence is SEQ ID NO: 101. The amino acid sequences of CDR1, CDR2 and CDR3 within the light chain variable region of the mouse 7B4 antibody are SEQ ID NO: 17, SEQ ID NO: 18 and SEQ ID NO: 19, respectively.

The nucleic acid sequence of the light chain variable region of the mouse 8C11 antibody is SEQ ID NO: 102, and the amino acid sequence is SEQ ID NO: 103. The amino acid sequences of CDR1, CDR2 and CDR3 within the light chain variable region of the mouse 8C11 antibody are SEQ ID NO: 23, SEQ ID NO: 24 and SEQ ID NO: 25, respectively.

The nucleic acid sequence of the light chain variable region of the mouse 10C3 antibody is SEQ ID NO: 104, and the amino acid sequence is SEQ ID NO: 105. The amino acid sequences of CDR1, CDR2 and CDR3 within the light chain variable region of the mouse 10C3 antibody are SEQ ID NO: 29, SEQ ID NO: 30 and SEQ ID NO: 31, respectively.

The nucleic acid sequence of the light chain variable region of the mouse 11D10 antibody is SEQ ID NO: 106, and the amino acid sequence is SEQ ID NO: 107. The amino acid sequences of CDR1, CDR2 and CDR3 within the light chain variable region of the mouse 11D10 antibody are SEQ ID NO: 29, SEQ ID NO: 30 and SEQ ID NO: 31, respectively.

The nucleic acid sequence of the light chain variable region of the mouse 13D4 antibody is SEQ ID NO: 108, and the amino acid sequence is SEQ ID NO: 109. The amino acid sequences of CDR1, CDR2 and CDR3 within the light chain variable region of the mouse 13D4 antibody are SEQ ID NO: 35, SEQ ID NO: 36 and SEQ ID NO: 37, respectively.

The nucleic acid sequence of the light chain variable region of the mouse 13H11 antibody is SEQ ID NO: 100, and the amino acid sequence is SEQ ID NO: 111. The amino acid sequences of CDR1, CDR2 and CDR3 within the light chain variable region of the mouse 13H11 antibody are SEQ ID NO: 41, SEQ ID NO: 42 and SEQ ID NO: 43, respectively.

The nucleic acid sequence of the light chain variable region of the mouse 14C1 antibody is SEQ ID NO: 112, and the amino acid sequence is SEQ ID NO: 113. The amino acid sequences of CDR1, CDR2 and CDR3 within the light chain variable region of the mouse 14C1 antibody are SEQ ID NO: 41, SEQ ID NO: 42 and SEQ ID NO: 43, respectively.

Examples of more preferred monoclonal antibodies in the present invention can include monoclonal antibodies produced by hybridomas mp5B7, mp7B4, mp13D4 and mp13H11.

Hybridomas mp5B7, mp7B4, mp13D4 and mp13H11 were accepted by National Institute of Technology and Evaluation, International Patent Organism Depositary, under accession No. NITE ABP-1211, NITE ABP-1212, NITE ABP-1213 and NITE ABP-1214 as of Jan. 27, 2012. The details specifying the deposition will be described as follows.

(1) Name and Address of Depositary Authority Name: National Institute of Technology and Evaluation, Advanced Industrial Science and Technology, International Patent Organism Depositary Address: 2-5-8 Kazusa Kamatari Kisarazu-shi, Chiba Ibaraki, 292-0818, Japan

### (2) Deposit Date: Jan. 27, 2012

(3) Deposit number NITE BP-1211 (hybridoma mp5B7)

NITE BP-1212 (hybridoma mp7B4)

NITE BP-1213 (hybridoma mp13D4)

NITE BP-1214 (hybridoma mp13H11)

In particular, more preferred antibodies are an antibody having a combination of

the heavy chain CDR1: DYNLH, CDR2: YIYPYNGNTGYNQKFKR, and CDR3: GGIYDDYYDYAIDY, and
the light chain CDR1: RASENIYSHIA, CDR2: GATNLAH, and CDR3: QHFWGTP
as the sequences of CDRs constituting its variable regions; an antibody having a combination of
the heavy chain CDR1: SHYYWT, CDR2: YISYDGSNNYNPSLKN, and CDR3: EGPLYYGNPYWYFDV, and
the light chain CDR1: RASQDIDNYLN, CDR2: YTSRLHS, and CDR3: QQFNTLP
as the sequences of CDRs constituting its variable regions; and
an antibody having a combination of
the heavy chain CDR1: SHYYWS, CDR2: YISYDGSNNYNPSLKN, and CDR3: EGPLYYGNPYWYFDV, and
the light chain CDR1: RASQDIDNYLN, CDR2: YTSRLHS, and CDR3: QQFNTLP,
as the sequences of CDRs constituting its variable regions.

A chimeric antibody or a humanized antibody recognizing PLD4 can be produced by genetic engineering using a polynucleotide encoding it. As described in Patent Document 1, for example, each active chimeric antibody (ch3B4Ab, ch5B7Ab, ch7B4Ab, ch8C11Ab, ch10C3Ab, ch11D10Ab, ch13D4Ab, ch13H11Ab, ch14C1Ab, ch11G9.6Ab etc.) can be easily produced using each CDR region of the above mouse monoclonal antibodies (3B4, 5B7, 7B4, 8C11, 10C3, 11D10, 13D4, 13H11, 14C1, 11G9.6 etc.) by those of skill in the art.

The present inventors have verified that monoclonal antibodies against PLD4 have CDC (Complement Dependent Cytotoxicity) activity and ADCC (Antibody-dependent cellular cytotoxicity) activity against the PLD4-expressing cells. Therefore, the anti-PLD4 monoclonal antibodies according to the present invention have cytotoxicity action against PLD4-expressing cells.

That is, the present invention relates to an agent for suppressing activated B cells, wherein the agent comprises an antibody binding to an extracellular domain of PLD4 as an active component. Alternatively, the present invention provides a method for suppressing antibody production, the method including a step of administering an antibody binding to an extracellular domain of PLD4. The present invention further relates to use of an antibody binding to an extracellular domain of PLD4 in production of a pharmaceutical composition for suppressing activated B cells.

In the present invention, an antibody modified as needed can be used. According to the present invention, an antibody recognizing the extracellular domain of PLD4 has the activated B cell-suppressing action. That is, it has been believed that there is a possibility that an antibody itself have cytotoxicity action against activated B cells. The subclass of an antibody showing intense effector action is known. Alternatively, suppressive effect on activated B cells can be further increased by modifying an antibody with a cytotoxic agent. As the cytotoxic agents, the following substances can be mentioned.

Toxins: Pseudomonas Endotoxin (PE), diphtheria toxin, lysine

### Radioisotopes: Tc99m, Sr89, I131, Y90

Anticancer agents: calicheamicin, mitomycin, paclitaxel

The toxins containing proteins can be bound to an antibody or a fragment thereof or the like by a bifunctional reagent. Alternatively, by conjugating a gene encoding an antibody with a gene encoding a toxin, a fusion protein of the two can be also obtained. A method for binding a radioisotope to an antibody is also known. A method for labeling an antibody with a radioisotope, for example, using a chelating agent is known. Further, an anticancer agent can be bound to an antibody, using glycan or a bifunctional reagent or the like.

In the present invention, an antibody whose structure is artificially modified can be used as an active component. For example, various modification methods for improving the cytotoxicity action and stability of antibodies are known. Concretely, immunoglobulin in which the glycan of its heavy chain is modified is known (Shinkawa, T. et al. J. Biol. Chem. 278:3466-3473. 2003). By modification of glycan, the ADCC (Antibody Dependent Cell-mediated Cytotoxicity) activity of immunoglobulin was increased.

In the present invention, one or more monoclonal antibodies can be used. For example, several types of monoclonal antibodies recognizing the extracellular domain of PLD4 can be combined and used for the present invention.

As described below, it can be verified that anti-PLD4 antibodies have suppressive action on the acquired immune antibody-producing activity of activated B cells. B cells produce a large amount of antibodies by stimulation of a BCR ligand or a TLR ligand (preferably TLR4 ligand, TLR7 ligand or TLR9 ligand). An anti-PLD4 antibody is provided before and after stimulation of the above ligand on B cells or simultaneously with stimulation of the ligand, and using B cells for which an anti-PLD4 antibody is not provided as a control, ability to produce acquired immune antibodies derived from B cells is compared. The antibody-producing ability can be evaluated by measuring secretory immunoglobulin contained in a culture supernatant of B cells. As a result of the comparison, when the amount of the acquired immune antibody derived from B cells in the supernatant significantly declines by adding an anti-PLD4 antibody, it can be verified that the tested anti-PLD4 antibody has suppressive action on the antibody-producing ability of B cells. A method for measuring the antibodies is known. B cells are cells which produce hormonal immunity (secretory antibody) in a living body. Therefore, hormonal immunity can be adjusted by suppressing the antibody-producing ability of B cells.

When an antibody recognizing the extracellular domain of PLD4 is administered to a host different from an organism species from which the antibody is derived, it is desired to process into a form which is difficult to be recognized as a foreign substance by such a host. By processing into molecules described below, for example, it can be difficult that immunoglobulin is recognized as a foreign substance. Techniques for processing immunoglobulin molecules as described below are known:
a fragment containing an antigen-binding region which lacks a constant region (Monoclonal Antibodies: Principles and Practice. third edition, Academic Press Limited. 1995; Antibody Engineering, A Practical Approach, IRL PRESS, 1996);
a chimeric antibody constituted of an antigen-binding region of a monoclonal antibody and a constant region of host immunoglobulin (Experimental manual for genetic expression, Kodansha Ltd. 1994 (edited by Isao Ishida and Tamie Ando)); and
a CDR-substituted antibody in which a complementarity-determining region (CDR) in host immunoglobulin is substituted by the CDR of a monoclonal antibody (Experimental manual for genetic expression, Kodansha Ltd. 1994 (edited by Isao Ishida and Tamie Ando)).

Alternatively, a variable region gene of human immunoglobulin can be also obtained by the phage display method (McCafferty J. et al., Nature 348:552-554, 1990; Kretzschmar T et. al., Curr Opin Biotechnol. 2002 December; 13(6):598-602.). In the phage display method, a gene encoding a variable region of human immunoglobulin is incorporated into a phage gene. Using various types of immunoglobulin genes as sources, a phage library can be also created. A phage expresses such a variable region as a fusion protein of a protein constructing the phage itself. The variable region expressed by the phage on the phage surface maintains binding activity to antigens. Therefore, by selecting a phage binding to an antigen or cells expressing the antigen or the like, a phage expressing a variable region having target binding activity can be screened from a phage library. Further, a gene encoding a variable region having target binding activity is maintained in the phage particle selected as above. That is, in the phage display method, using the binding activity of a variable region as an index, a gene encoding a variable region having target binding activity can be obtained.

In the agent for suppressing B cell activity or the method for suppressing B cell activity according to the present invention, an antibody recognizing the extracellular domain of PLD4 or an antibody fragment containing at least the antigen-binding region thereof can be administered as a protein or a polynucleotide encoding it. In order to administer a polynucleotide, it is desired that a vector in which a polynucleotide encoding a target protein is arranged be used under control of a proper promoter so that the target protein can be expressed. In a vector, an enhancer and a terminator can be also arranged. Vectors which maintain the genes of heavy and light chains constituting immunoglobulin and in which an immunoglobulin molecule can be expressed are known. A vector in which immunoglobulin can be expressed can be administered by introduction into cells. For administration to a living body, a vector which can infect cells by administration to the living body can be directly administered. Alternatively, a vector is introduced into a lymphocyte separated from a living body and then the vector can be returned into the living body (ex vivo).

In the agent for suppressing B cell activity or the method for suppressing B cell activity based on the present invention, the amount of monoclonal antibody to be administered to a living body is normally 0.5 mg to 10 mg, for example 1 mg to 50 mg, preferably 2 mg to 10 mg as immunoglobulin per kg of body weight. An interval of administration of an antibody to a living body can be properly adjusted in order that an effective concentration of immunoglobulin in the living body during treatment period can be maintained. Concretely, for example, an antibody can be administered at intervals of 1 to 2 weeks. Any administration route can be used. Those of skill in the art can properly select an effective administration route for treatment. Concretely, oral or parenteral administration can be mentioned. By an intravenous injection, an intramuscular injection, an intraperitoneal injection or a subcutaneous injection or the like, for example, an antibody can be systemically or locally administered. The formulations suitable for parenteral administration in the present invention include injections, suppositories, sprays and the like. In addition, when provided to cells, immunoglobulin is provided in a culture fluid in an amount of normally 1 μg/ml, preferably 10 μg/mL or more, more preferably 50 μg/mL or more, and further preferably 0.5 mg/mL or more.

In the agent for suppressing B cell activity or the method for suppressing B cell activity based on the present invention, a monoclonal antibody can be administered to a living body by any method. A monoclonal antibody is normally combined with a pharmaceutically acceptable carrier. A monoclonal antibody can be combined with additives as needed, such as a thickener, a stabilizer, an antiseptic and a solubilizing agent. Such carriers or additives include lactose, a citric acid, a stearic acid, magnesium stearate, sucrose, starch, talc, gelatin, agar, plant oil, ethylene glycol and the like. The term “pharmaceutically acceptable” means to be approved by government authorities of various countries, or that its use for animals, mammals and, in particular, human is listed in pharmacopoeias of various countries or pharmacopoeias commonly acknowledged. The agent for suppressing B cell activity in the present invention can be also supplied in the form of freeze-drying powders or tablets at one or more doses. Further, sterilized water for injections, a physiological salt solution or a buffer solution, which are used for dissolution, can be combined with freeze-drying powders or tablets in order that the composition will obtain a desired concentration before administration.

Further, for administration as a vector expressing immunoglobulin, a heavy chain and a light chain are cotransfected as different plasmids and each plasmid can be administered at 0.1 to 10 mg, for example 1 to 5 mg per kg of body weight. In addition, 1 to 5 μg vectors/10⁶ cells are used to introduce into cells in vitro. The present invention will be now described in more detail by way of examples.

All of the related art literatures cited in the present description are incorporated by reference herein.

The present invention will be now described in more detail by way of examples. It should be noted, however, that the present invention is not limited to the examples.

### EXAMPLES

### Example 1

Human PBMC (1×10⁷ cells/ml) was stimulated by CpG2006, a ligand of TLR9, (a final concentration of 1 μM) and incubated in a 24 well plate in a CO₂ incubator (37° C., 5% CO₂) for about 20 hours. In parallel, human PBMC (1×10⁷ cells/ml) which was not stimulated was also cultured in a CO₂ incubator (37° C., 5% CO₂) for about 20 hours.

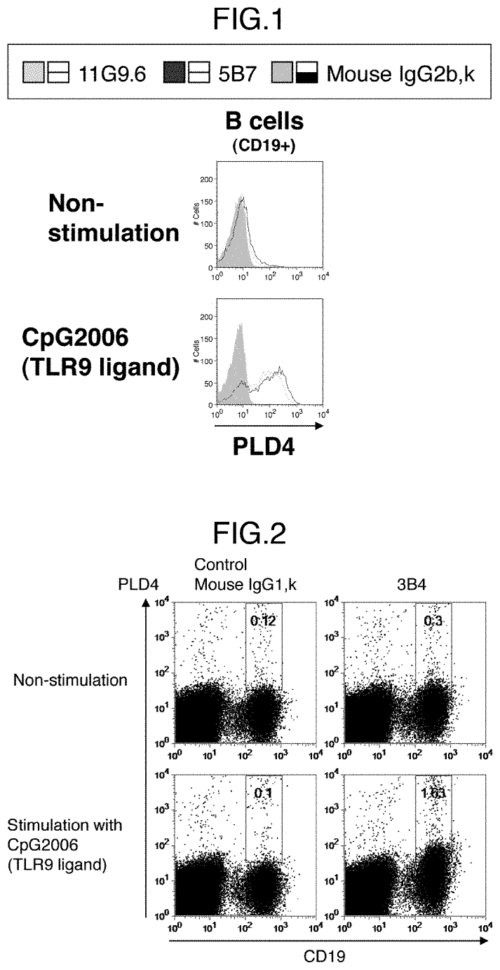

Human PBMC was treated with FcR Blocking Reagent (Miltenyi), which was diluted 5-fold with FACS buffer (1% FBS/PBS), at 4° C. for 20 minutes. After washing, staining was carried out with 5B7, 11G9.6 or mouse IgG2b, [symbol] , a primary antibody, (each 10 μg/ml) at 4° C. for 15 minutes. A secondary antibody and subsequent antibodies were diluted with FACS buffer so that FcR Blocking Reagent would be diluted 25-fold. PE-labeled anti-mouse Ig (BD), a secondary antibody, was diluted 100-fold and the solution was added thereto and mixed. Besides, to fractionate B cells on FACS, an APC-labeled anti-human CD 19 antibody (Biolegend) was diluted 30-fold with FACS buffer containing FcR Blocking Reagent and staining was carried out at 4° C. for 15 minutes. Using FACS Calibur (BD), data was incorporated. Living cells were gated on a dot plot of the X axis: FSC and the Y axis: SSC. Data was incorporated until the number of cells in the living cell gate became 100,000 counts. B cells: anti-marker molecule antibody-positive cells were gated. The gated cells were analyzed on the histogram with the X axis: PLD4, and the results of staining with mouse IgG2b, [symbol] were overlaid thereon. Consequently, anti-PLD4 antibodies were hardly bound to non-stimulated, but were selectively bound to activated B cells by stimulation with TLR9 ligand (FIG. 1). This shows that PLD4 is expressed on activated B cells.

### Example 2

### <Binding Test to B Cells by Each Monoclonal Antibody>

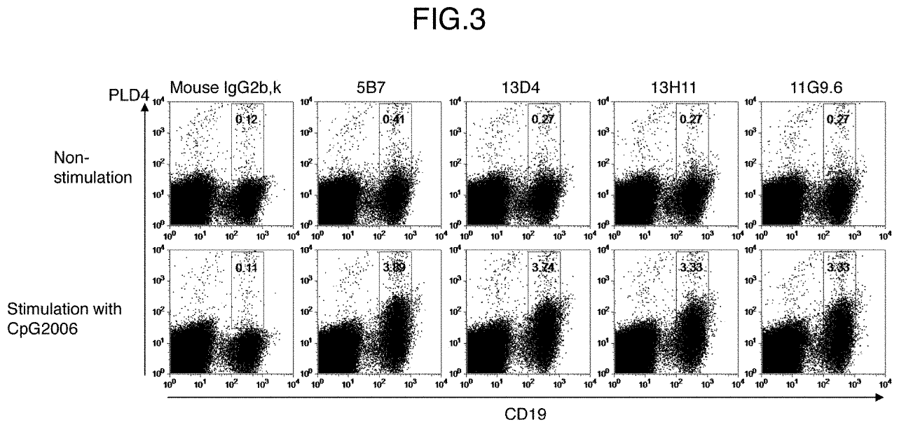

Human PBMC was stimulated with CpG2006 with a final concentration of 1 μM for about 20 hours. Cells were collected and treated with FcR Blocking Reagent at 4° C. for 20 minutes. After washing, staining was carried out with each 10 μg/ml of 3B4, 5B7, 13D4, 13H11, 11G9.6, mouse IgG1, [symbol] or mouse IgG2b, [symbol] , a primary antibody, at 4° C. for 15 minutes. Staining was carried out with PE-labeled anti-mouse Ig, a secondary antibody, at 4° C. for 15 minutes. For gating of a B cell group, double staining was carried out with an APC-labeled anti-human CD19 antibody at 4° C. for 15 minutes. A living cell group on the dot plot of the X axis: FSC and the Y axis: SSC was analyzed by binding of anti-PLD4 antibody to CD19+ B cells (FIG. 2 and FIG. 3). Consequently, all of the tested anti-PLD4 monoclonal antibodies were bound to B cells stimulated by TLR9. That is, it was verified that by all anti-PLD4 monoclonal antibodies, expression of PLD4 was induced in B cells in an activation-dependent manner.

### Example 3

### <Cytotoxic Activity of Anti-PLD4 Chimeric Antibodies Against Activated B Cells>

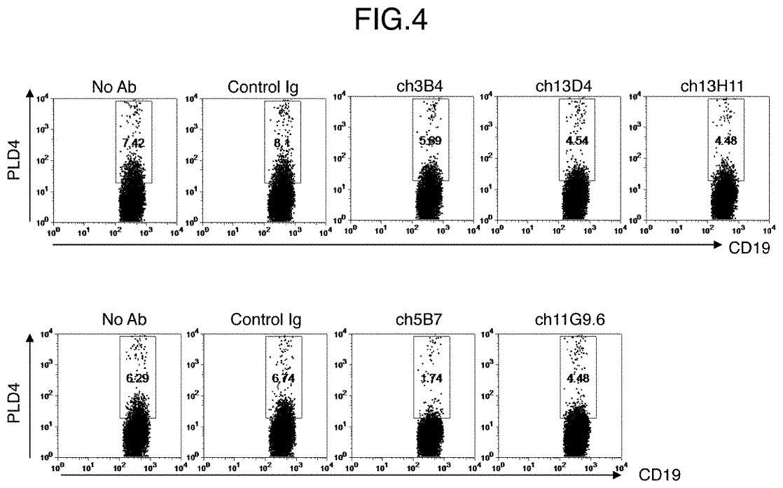

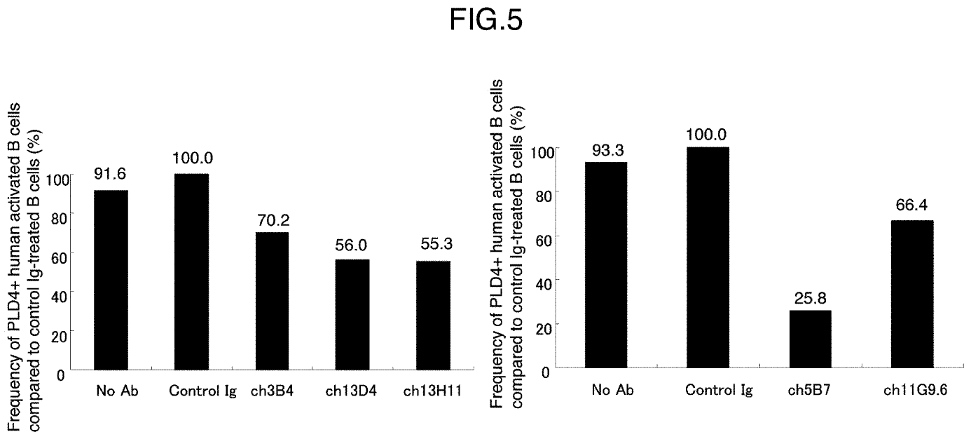

Frequency of PLD4+ activated B cells induced by stimulation with TLR9 ligand (1 μM) was used as an index. Human PBMC was cultured with CpG2006 and each anti-PLD4 chimeric antibody or control Ig for about 16 hours. As a medium, RPM11640 (SIGMA) was used (including 10% FBS (Equitech-bio), 5 ml of 200 mM L-Glutamine (GIBCO), 5 ml of Pen-Strep (GIBCO), 5 ml of Sodium Pyruvate (GIBCO), and 0.5 ml of 50 mM 2-ME (SIGMA)). The cells were collected and treated with FcR Blocking Reagent at 4° C. for 20 minutes. After washing, the cells were further stained by 5B7 or 13D4, 3B4 or mouse IgG2b, [symbol] , a primary antibody, at 4° C. for 15 minutes (each 10 μg/ml). A sample in which PBMC was treated with a chimeric 3B4 antibody (ch3B4), a chimeric 3D4 antibody (ch3D4), or a chimeric 13H11 antibody (ch13H11) was stained with 5B7, and a sample in which PBMC was treated with a chimeric 5B7 antibody (ch5B7) or a chimeric 11G9.6 antibody (ch11G9.6) was stained with 13D4. It has been verified that an anti-PLD4 antibody clone treated for ADCC and an anti-PLD4 antibody clone used for staining do not compete with each other. The binding of the anti-PLD4 was found by PE-labeled anti-mouse Ig, a secondary antibody, at 4° C. for 15 minutes. For gating of B cells, double staining was carried out with an APC-labeled anti-human CD19 antibody at 4° C. for 15 minutes (FIG. 4). The population of PLD4+ activated B cells treated with each chimeric anti-PLD4 antibody was compared with that of PLD4+ activated B cells treated with the control antibody (FIG. 5). Consequently, all of the chimeric anti-PLD4 antibodies reduced activated PLD4+ B cells compared to the treatment with control Ig (when a case of treating with control Ig was considered as 100%, ch3B4: 70.2%, ch13D4: 56.0%, ch13H11: 55.3%, ch5B7: 25.8%, ch11G9.6: 66.4%).

### Example 4

### <Inhibitory Effects of the Chimeric Anti-PLD4 Antibodies Against Activated B Cells>

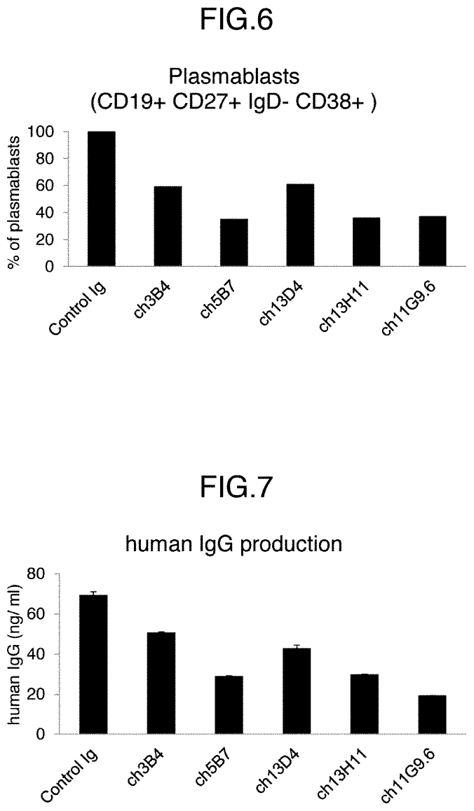

To determine the effect of anti-human PLD4 antibody on B cells maturation and Ig production through B cell activation, whole human PBMCs were treated with ch3B4, ch5B7, ch13D4, ch13H11, ch11G9.6, or control Ig for 24 h. Then, the PBMCs were further cultured in the presence of CpG2216 (1 μM) and recombinant human IL-6 to induce B cell activation, resulting in B cell maturation. In the result of culture of activated B cells for 7 days, Plasmablasts, CD19+CD27+IgD−CD38+, in the activated B cells was analyzed by flow cytometry with a PE-labeled anti-human CD19 antibody. In order to measure human IgG production, the cultured activated B cells were re-stimulated with 50 ng/ml of PMA (Phorbol myristate acetate) after washed with PBS 2 times. Two days later, human IgG production was measured in the culture supernatants by ELISA. Plasmablasts in the activated B cells were reduced by the treatment with ch3B4, ch5B7, ch13D4, ch13H11, or ch11G9.6 compared with control Ig treatment (FIG. 6). Also, human IgG production was reduced by the treatment with ch3B4, ch5B7, ch13D4, ch13H11, or ch11G9.6 compared to control Ig treatment (FIG. 7). These results indicated that the treatment with the chimeric anti-human PLD4 Abs reduced Ab-secreting activated human B cells.

### INDUSTRIAL APPLICABILITY

As shown in the above examples, anti-PLD4 antibodies recognize and suppress activated B cells. Therefore, the antibodies are useful for prevention and treatment of diseases involved in immune function (autoimmune diseases and allergic diseases).

### <Explanation of Sequence Information of Anti-PLD4 Monoclonal Antibodies According to the Present Invention>

### 1. Anti-PLD4 Mouse 11G9.6 Antibody

The nucleic acid sequence of the heavy chain variable region of the obtained anti-PLD4 mouse 11G9.6 antibody is SEQ ID NO: 74, and the amino acid sequence is SEQ ID NO: 75. The amino acid sequences of CDR1, CDR2 and CDR3 within the heavy chain variable region of the mouse 11G9.6 antibody are SEQ ID NO: 2, SEQ ID NO: 3 and SEQ ID NO: 4, respectively.

The nucleic acid sequence of the heavy chain variable region of the anti-PLD4 mouse 11G9.6 antibody (504 bp) [capital letters: mouse 11G9.6 VH variable region, small letters: mouse IgG2b heavy chain constant region] (SEQ ID NO: 74)

ATGAGATCACAGTTCTCTATACAGTTACTGAGCACACAGAACCTCACCTT
GGGATGGAGCTGTATCATCCTCTTCTTGGTAGCAACAGCTACAGGTGTCC
ACTCCCAGGTCCAACTGCAGCAGCCTGGGGCTGAACTGGTGAAGCCTGGG
ACTTCAGTGAAAATGTCCTGCAAGGCTTCTGGCTACACCTTCACCAGCTA
CTGGATGCACTGGGTGAAGCAGAGGCCGGGACAAGGCCTTGAGTGGATTG
GAGATATTTATCCTGGTAGTGATAGTACTAACTACAATGAGAAGTTCAAG
AGCAAGGCCACACTGACTGTAGACACATCCTCCAGCACAGCCTACATGCA
ACTCAGCAGCCTGACATCTGAGGACTCTGCGGTCTATTACTGTGCAAGAG
GAGGGTGGTTGGATGCTATGGACTACTGGGGTCAAGGAACCTCAGTCACC
GTCTCCTCAgccaaaacaacacccccatcagtctatccactggcccctaa
gggc

The amino acid sequence of the heavy chain variable region of the mouse 11G9.6 antibody (168 a. a.) [capital letters: mouse 11G9.6 VH variable region, small letters: mouse IgG2b heavy chain constant region] The underlined sequence shows its signal sequence and the double underline shows its CDR regions (CDR1, CDR2 and CDR3) (SEQ ID NO: 75).

MRSQFSIQLLSTQNLTLGWSCHLFLVATATGVIISQVQLQQPGAFLVKPG
TSVKMSCKASGYTFTSYWMHWVKQRPGQGLEWIGDIYPGSDSTNYNEKFK
SKATLTVDTSSSTAYMQLSSLTSEDSAVYYCARGGWLDAMDYWGQGTSVT
VSSakttppsvyplapkg

CDR1 in the heavy chain variable region of the 11G9.6 antibody

### (SEQ ID NO: 2)

CDR2 in the heavy chain variable region of the 11G9.6 antibody

### (SEQ ID NO: 3)

CDR3 in the heavy chain variable region of the 11G9.6 antibody

### (SEQ ID NO: 4)

The nucleic acid sequence of the light chain variable region of the obtained anti-PLD4 mouse 11G9.6 antibody is SEQ ID NO: 38, and the amino acid sequence is SEQ ID NO: 39. The amino acid sequences of CDR1, CDR2 and CDR3 within the light chain variable region of the mouse 11G9.6 antibody are SEQ ID NO: 40, SEQ ID NO: 41 and SEQ ID NO: 42, respectively.

The nucleic acid sequence of the light chain variable region of the anti-PLD4 mouse 11G9.6 antibody (421 bp) [capital letters: mouse 11G9.6 VL variable region, small letters: mouse Ig [symbol] light chain constant region] (SEQ ID NO: 94)

ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGAGACAGAGTCACCATCAGTTGCAGGGCAAGTCAGGACATTAGC
AATTATTTAAACTGGTATCAGCAGAAACCAGATGGAACTGTTAAACTCCT
GATCTACTACACATCAAGATTACACTCAGGAGTCCCATCAAGGTTCAGTG
GCAGTGGGTCTGGAACAGATTATTCTCTCACCATTAGCAACCTGGAGCAA
GAAGATATTGCCACTTACTTTTGCCAACAGGGTAATACGCTTCCGTGGAC
GTTCGGTGGAGGCACCAAGCTGGAAATCAAAcgggctgatgctgcaccaa
ctgtatccatcaagggcgaat

The amino acid sequence of the light chain variable region of the mouse 11G9.6 antibody (140 a. a.) [capital letters: mouse 11G9.6 VL variable region, small letters: mouse Ig [symbol] light chain constant region] The underlined sequence shows its signal sequence and the double underline shows its CDR regions (CDR1, CDR2 and CDR3) (SEQ ID NO: 95).

MMSSAQFLGLLLLCFQGTRCDIQMTQTTSSLSASLGDRVTISCRASQDIS
NYLNWYQQKPDGTVKLLIYYTSRLHSGVPSRFSGSGSGTDYSLTISNLEQ
EDIATYFCQQGNTLPWTFGGGTKLEIKradaaptvsikge

CDR1 in the light chain variable region of the 11G9.6 antibody

### (SEQ ID NO: 5)

CDR2 in the light chain variable region of the 11G9.6 antibody

### (SEQ ID NO: 6)

CDR3 in the light chain variable region of the 11G9.6 antibody

### (SEQ ID NO: 7)

### 2. Anti-PLD4 Mouse 3B4 Antibody

The nucleic acid sequence of the heavy chain variable region of the obtained anti-PLD4 mouse 3B4 antibody is SEQ ID NO: 76, and the amino acid sequence is SEQ ID NO: 77. The amino acid sequences of CDR1, CDR2 and CDR3 within the heavy chain variable region of the mouse 3B4 antibody are SEQ ID NO: 8, SEQ ID NO: 9 and SEQ ID NO: 10, respectively.

The nucleic acid sequence of the heavy chain variable region of the anti-PLD4 mouse 3B4 antibody (437 bp) [capital letters: mouse 3B4 VH variable region, small letters: mouse IgG1 heavy chain constant region]

ATGGAATGTAACTGGATACTTCCTTTTATTCTGTCGGTAATTTCAGGGGT
CTCCTCAGAGGTTCAGCTCCAGCAGTCTGGGACTGTGCTGTCAAGGCCTG
GGGCTTCCGTGACGATGTCCTGCAAGGCTTCTGGCGACAGCTTTACCACC
TACTGGATGCACTGGGTAAAACAGAGGCCTGGACAGGGTCTAGAATGGAT
TGGTGCTATCTATCCTGGAAATAGTGAAACTAGCTACAACCAGAAGTTCA
AGGGCAAGGCCAAACTGACTGCAGTCACATCCGCCAGCACTGCCTATATG
GAGTTCACTAGCCTGACAAATGAGGACTCTGCGGTCTATTACTGTACGGG
GGGTTATTCCGACTTTGACTACTGGGGCCAAGGCACCACTCTCACAGTCT
CCTCAgccaaaacgacacccccatctgtctatccact

The amino acid sequence of the heavy chain variable region of the mouse 3B4 antibody (145 a. a.) [capital letters: mouse 3B4 VH variable region, small letters: mouse IgG1 heavy chain constant region] The underlined sequence shows its signal sequence and the double underline shows its CDR regions (CDR1, CDR2 and CDR3).

MECNWILPFILSVISGVSSEVQLQQSGTVLSRPGASVTMSCKASGDSFTT
YWMHWVKQRPGQGLEWIGAIYPGNSETSYNKFKGKAKLTAVTSASTAYME
FTSLTNEDSAVYYCTGGYSDFDYWGQGTTLTVSSakttppsvyp

CDR1 in the heavy chain variable region of the 3B4 antibody

### TYWMH

CDR2 in the heavy chain variable region of the 3B4 antibody

### AIYPGNSETSYNQKFKG

CDR3 in the heavy chain variable region of the 3B4 antibody

### GYSDFDY

The nucleic acid sequence of the light chain variable region of the obtained anti-PLD4 mouse 3B4 antibody is SEQ ID NO: 96, and the amino acid sequence is SEQ ID NO: 97. The amino acid sequences of CDR1, CDR2 and CDR3 within the light chain variable region of the mouse 3B4 antibody are SEQ ID NO: 11, SEQ ID NO: 12 and SEQ ID NO: 13, respectively.

The nucleic acid sequence of the light chain variable region of the anti-PLD4 mouse 3B4 antibody (459 bp) [capital letters: mouse 3B4 VL variable region, small letters: mouse Ig [symbol] light chain constant region]

ATGATGGTCCTTGCTCAGTTTCTTGCATTCTTGTTGCTTTGGTTTCCAGG
TGCAGGATGTGACATCCTGATGACCCAATCTCCATCCTCCATGTCTGTAT
CTCTGGGAGACACAGTCAGCATCACTTGCCATGCAAGTCAGGGCATTAGA
AGTAATATAGGGTGGTTGCAGCAGAAACCAGGGAAATCATTTAAGGGCCT
GATCTTTCATGGAACCAACTTGGAAGATGGAGTTCCATCAAGGTTCAGTG
GCAGAGGATCTGGAGCAGATTATTCTCTCACCATCAACAGCCTGGAATCT
GAAGATTTTGCAGACTATTACTGTGTACAGTATGTTCAGTTTCCTCCAAC
GTTCGGCTCGGGGACAAAGTTGGAAATAAGAcgggctgatgctgcaccaa
ctgtatccatcttcccaccatccagtgagcagttaacatctggaggtgcc
tcagtcgtg

The amino acid sequence of the light chain variable region of the mouse 3B4 antibody (153 a. a.) [capital letters: mouse 3B4 VL variable region, small letters: mouse Ig [symbol] light chain constant region] The underlined sequence shows its signal sequence and the double underline shows its CDR regions (CDR1, CDR2 and CDR3).

MMVLAQFLAFLLLWFPGAGCDILMTQSPSSMSVSLGDTVSITCHASQGIR
SNIGWLQQKPGKSFKGLIFHGTNLEDGVPSRFSGRGSGADYSLTINSLES
EDFADYYCVQYVQFPPTFGSGTKLEIRradaaptvsifppsseqltsgga
svv

CDR1 in the light chain variable region of the 3B4 antibody

### HASQGIRSNIG

CDR2 in the light chain variable region of the 3B4 antibody

### HGTNLED

CDR3 in the light chain variable region of the 3B4 antibody

### VQYVQFP

### 3. Anti-PLD4 Mouse 5B7 Antibody

The nucleic acid sequence of the heavy chain variable region of the obtained anti-PLD4 mouse 5B7 antibody is SEQ ID NO: 78, and the amino acid sequence is SEQ ID NO: 79. The amino acid sequences of CDR1, CDR2 and CDR3 within the heavy chain variable region of the mouse 5B7 antibody are SEQ ID NO: 14, SEQ ID NO: 15 and SEQ ID NO: 16, respectively.

The nucleic acid sequence of the heavy chain variable region of the anti-PLD4 mouse 5B7 antibody (475 bp) [capital letters: mouse 5B7 VH variable region, small letters: mouse IgG2b heavy chain constant region]

ATGGGATGGAGCTGGATCTTTCTCTTCCTCCTGTCAGGAACTGCAGGCGT
CCACTCTGAGGTCCAGCTTCAGCAGTCAGGACCTGAACTGGTGAAACCTG
GGGCCTCAGTGAAGATATCCTGCAAGGCTTCTGGATACACATTCACTGAC
TACAACTTGCACTGGGTGAAGCAGAGCCATGGAAAGAGCCTTGAGTGGAT
TGGATATATTTATCCTTACAATGGTAATACTGGCTACAACCAGAAGTTCA
AGAGGAAGGCCACATTGACTGTAGACAATTCCTCCGGCACAGTCTACATG
GAGCTCCGCAGCCTGACATCTGAGGACTCTGCAGTCTATTACTGTGCAAG
AGGAGGGATCTATGATGATTACTACGACTATGCTATCGACTATTGGGGTC
AAGGAACCTCAGTCACCGTCTCCTCAgccaaaacaacacccccatcagtc
tatccactggcccctaagggcgaat

The amino acid sequence of the heavy chain variable region of the mouse 5B7 antibody (158 a. a.) [capital letters: mouse 5B7 VH variable region, small letters: mouse IgG2b heavy chain constant region] The underlined sequence shows its signal sequence and the double underline shows its CDR regions (CDR1, CDR2 and CDR3).

MGWSWIFLELLSGTAGVHSEVQLQQSGPELVKPGASVKISCKASGYTFTD
YNLHWVKQSHGKSLEWIGYIYPYNGNTGYNKFKRKATLTVDNSSGTVYME
LRSLTSEDSAVYYCARGGIYDDYYDYAIDYWGQGTSVTVSSakttppsvy
plapkge

CDR1 in the heavy chain variable region of the 5B7 antibody

### DYNLH

CDR2 in the heavy chain variable region of the 5B7 antibody

### YIYPYNGNTGYNQKFKR

CDR3 in the heavy chain variable region of the 5B7 antibody

### GGIYDDYYDYAIDY

The nucleic acid sequence of the light chain variable region of the obtained anti-PLD4 mouse 5B7 antibody is SEQ ID NO: 98, and the amino acid sequence is SEQ ID NO: 99. The amino acid sequences of CDR1, CDR2 and CDR3 within the light chain variable region of the mouse 5B7 antibody are SEQ ID NO: 17, SEQ ID NO: 18 and SEQ ID NO: 19, respectively.

The nucleic acid sequence of the light chain variable region of the anti-PLD4 mouse 5B7 antibody (467 bp) [capital letters: mouse 5B7 VL variable region, small letters: mouse Ig [symbol] light chain constant region]

ATGAGTGTGCCCACTCAGGTCCTGGGGTTGCTGCTGCTGTGGCTTACAGA
TGCCAGATGTGACATCCAGATGACTCAGTCTCCAGCCTCCCTATCTGTAT
CTGTGGGAGAAACTGTCGCCATCACATGTCGAGCAAGTGAGAATATTTAC
AGTCATATAGCATGGTATCAGCAGAAAGAGGGAAAATCTCCTCAGCGCCT
GGTCTATGGTGCAACAAACTTAGCACATGGTGTGCCATCAAGGTTCAGTG
GCAGTGGATCAGGCACACAGTATTCCCTCAAGATCAACAGCCTTCAGTCT
GAAGATTTTGGGAGTTATTACTGTCAACATTTTTGGGGTACTCCGTGGAC
GTTCGGTGGAGGCACCAAGCTGGAAATCAAAcgggctgatgctgcaccaa
ctgtatccatcttcccaccatccagtgagcagttaacatctggaggtgcc
tcagtcgtgtgcttctt

The amino acid sequence of the light chain variable region of the mouse 5B7 antibody (155 a. a.) [capital letters: mouse 5B7 VL variable region, small letters: mouse Ig [symbol] light chain constant region] The underlined sequence shows its signal sequence and the double underline shows its CDR regions (CDR1, CDR2 and CDR3).

MSVPTQVLGLLLLWLTDARCDIQMTQSPASLSVSVGETVAITCRASENIY
SHIAWYQQKEGKSPQRLVYGATNLAHGVPSRFSGSGSGTQYSLKINSLQS
EDFGSYYCQHFWGTPWTFGGGTKLEIKradaaptvsifppsseqltsgga
svvcf

CDR1 in the light chain variable region of the 5B7 antibody

### RASENIYSHIA

CDR2 in the light chain variable region of the 5B7 antibody

### GATNLAH

CDR3 in the light chain variable region of the 5B7 antibody

### QHFWGTP

### 4. Anti-PLD4 Mouse 7B4 Antibody

The nucleic acid sequence of the heavy chain variable region of the obtained anti-PLD4 mouse 7B4 antibody is SEQ ID NO: 80, and the amino acid sequence is SEQ ID NO: 81. The amino acid sequences of CDR1, CDR2 and CDR3 within the heavy chain variable region of the mouse 7B4 antibody are SEQ ID NO: 14, SEQ ID NO: 15 and SEQ ID NO: 16, respectively.

The nucleic acid sequence of the heavy chain variable region of the anti-PLD4 mouse 7B4 antibody (470 bp) [capital letters: mouse 7B4 VH variable region, small letters: mouse IgG2b heavy chain constant region]

ATGGGATGGAGCTGGATCTTTCTCTTCCTCCTGTCAGGAACTGCAGGCGT
CCACTCTGAGGTCCAGCTTCAGCAGTCAGGACCTGAACTGGTGAAACCTG
GGGCCTCAGTGAAGATATCCTGCAAGGCTTCTGGATACACATTCACTGAC
TACAACTTGCACTGGGTGAAGCAGAGCCATGGAAAGAGCCTTGAGTGGAT
TGGATATATTTATCCTTACAATGGTAATACTGGCTACAACCAGAAGTTCA
AGAGGAAGGCCACATTGACTGTAGACAATTCCTCCGGCACAGTCTACATG
GAGCTCCGCAGCCTGACATCTGAGGACTCTGCAGTCTATTACTGTGCAAG
AGGAGGGATCTATGATGATTACTACGACTATGCTATCGACTATTGGGGTC
AAGGAACCTCAGTCACCGTCTCCTCAgccaaaacaacacccccatcagtc
tatccactggcccctaaggg

The amino acid sequence of the heavy chain variable region of the mouse 7B4 antibody (156 a. a.) [capital letters: mouse 7B4 VH variable region, small letters: mouse IgG2b heavy chain constant region] The underlined sequence shows its signal sequence and the double underline shows its CDR regions (CDR1, CDR2 and CDR3).

MGWSWIFLFLLSGTAGVHSEVQLQQSGPELVKPGASVKISCKASGYTFTD
YNLHWVKQSHGKSLEWIGYIYPYNGNTGYNQKFKRKATLTVDNSSGTVYM
ELRSLTSEDSAVYYCARGGIYDDYYDYAIDYWGQGTSVTVSSakttppsv
yplapk

CDR1 in the heavy chain variable region of the 7B4 antibody

### DYNLH

CDR2 in the heavy chain variable region of the 7B4 antibody

### YIYPYNGNTGYNQKFKR

CDR3 in the heavy chain variable region of the 7B4 antibody

### GGIYDDYYDYAIDY

The nucleic acid sequence of the light chain variable region of the obtained anti-PLD4 mouse 7B4 antibody is SEQ ID NO: 100, and the amino acid sequence is SEQ ID NO: 101. The amino acid sequences of CDR1, CDR2 and CDR3 within the light chain variable region of the mouse 7B4 antibody are SEQ ID NO: 17, SEQ ID NO: 18 and SEQ ID NO: 19, respectively.

The nucleic acid sequence of the light chain variable region of the anti-PLD4 mouse 7B4 antibody (454 bp) [capital letters: mouse 7B4 VL variable region, small letters: mouse Ig [symbol] light chain constant region]

ATGAGTGTGCCCACTCAGGTCCTGGGGTTGCTGCTGCTGTGGCTTACAGA
TGCCAGATGTGACATCCAGATGACTCAGTCTCCAGCCTCCCTATCTGTAT
CTGTGGGAGAAACTGTCGCCATCACATGTCGAGCAAGTGAGAATATTTAC
AGTCATATAGCATGGTATCAGCAGAAAGAGGGAAAATCTCCTCAGCGCCT
GGTCTATGGTGCAACAAACTTAGCACATGGTGTGCCATCAAGGTTCAGTG
GCAGTGGATCAGGCACACAGTATTCCCTCAAGATCAACAGCCTTCAGTCT
GAAGATTTTGGGAGTTATTACTGTCAACATTTTTGGGGTACTCCGTGGAC
GTTCGGTGGAGGCACCAAGCTGGAAATCAAAcgggctgatgctgcaccaa
ctgtatccatcttcccaccatccagtgagcagttaacatctggaggtgcc
tcag

The amino acid sequence of the light chain variable region of the mouse 7B4 antibody (151 a. a.) [capital letters: mouse 7B4 VL variable region, small letters: mouse Ig [symbol] light chain constant region] The underlined sequence shows its signal sequence and the double underline shows its CDR regions (CDR1, CDR2 and CDR3).

MSVPTQVLGLLLLWLTDARCDIQMTQSPASLSVSVGETVAITCRASENIY
SHIAWYQQKEGKSPQRLVYGATNLAHGVPSRFSGSGSGTQYSLKINSLQS
EDFGSYYCQHFWGTPWTFGGGTKLEIKradaaptvsifppsseqltsgga
s

CDR1 in the light chain variable region of the 7B4 antibody

### RASENIYSHIA

CDR2 in the light chain variable region of the 7B4 antibody

### GATNLAH

CDR3 in the light chain variable region of the 7B4 antibody

### QHFWGTP

### 5. Anti-PLD4 Mouse 8C11 Antibody

The nucleic acid sequence of the heavy chain variable region of the obtained anti-PLD4 mouse 8C11 antibody is SEQ ID NO: 82, and the amino acid sequence is SEQ ID NO: 83. The amino acid sequences of CDR1, CDR2 and CDR3 within the heavy chain variable region of the mouse 8C11 antibody are SEQ ID NO: 20, SEQ ID NO: 21 and SEQ ID NO: 22, respectively.

The nucleic acid sequence of the heavy chain variable region of the anti-PLD4 mouse 8C11 antibody (462 bp) [capital letters: mouse 8C11 VH variable region, small letters: mouse IgG2b heavy chain constant region]

ATGGGATGGAGCTATATCATCCTCTTTTTGGTAGCAACAGCAACAGGGGT
CCACTCCCAGGTCCAACTGCAGCAGTCGGGGGCTGAACTGGTGAAGCCTG
GGGCTTCAGTGAAGTTGTCCTGCAAGGCTTCTGGCTACACCTTCACCAGC
TACTATTTGTACTGGGTGAGGCAGAGGCCTGGACAAGGCCTTGAGTGGAT
TGGACTGATTAATCCTACCAATAGTGATACTATCTTCAATGAGAAGTTCA
AGAGCAAGGCCACACTGACTGTAGACAAATCCTCCAGCACAGCATACATG
CAACTCAGCAGCCTGACATCTGAGGACTCTGCGGTCTATTACTGTACACG
AGAGGGGGGATATGGTTACGGCCCGTTTGCTTACTGGGGCCAAGGGACTC
TGGTCACTGTCTCTGCAgccaaaacaacacccccatcagtctatccactg
gcccctaagggc

The amino acid sequence of the heavy chain variable region of the mouse 8C11 antibody (154 a. a.) [capital letters: mouse 8C11 VH variable region, small letters: mouse IgG2b heavy chain constant region] The underlined sequence shows its signal sequence and the double underline shows its CDR regions (CDR1, CDR2 and CDR3).

MGWSYIILFLVATATGVHSQVQLQQSGAELVKPGASVKLSCKASGYTFTS
YYLYWVRQRPGQGLEWIGLINPTNSDTIFNEKFKSKATLTVDKSSSTAYM
QLSSLTSEDSAVYYCTREGGYGYGPFAYWGQGTLVTVSAakttppsvypl
apkg

CDR1 in the heavy chain variable region of the 8C11 antibody

### SYYLY

CDR2 in the heavy chain variable region of the 8C11 antibody

### LINPTNSDTIFNEKFKS

CDR3 in the heavy chain variable region of the 8C11 antibody

### EGGYGYGPFAY

The nucleic acid sequence of the light chain variable region of the obtained anti-PLD4 mouse 8C11 antibody is SEQ ID NO: 102, and the amino acid sequence is SEQ ID NO: 103. The amino acid sequences of CDR1, CDR2 and CDR3 within the light chain variable region of the mouse 8C11 antibody are SEQ ID NO: 23, SEQ ID NO: 24 and SEQ ID NO: 25, respectively.

The nucleic acid sequence of the light chain variable region of the anti-PLD4 mouse 8C11 antibody (457 bp) [capital letters: mouse 8C11 VL variable region, small letters: mouse Ig [symbol] light chain constant region]

ATGAAGTTGCCTGTTAGGCTGTTGGTGCTGATGTTCTGGATTCCTGCTTC
CAGCAGTGATGTTGTGATGACCCAAACTCCACTCTCCCTGCCTGTCAGTC
TTGGAGATCAAGCCTCCATCTCTTGCACATCTAGTCAGACCCTTGTACAC
AGTAATGGAAACACCTATTTACATTGGTACCTGCAGAAGCCAGGCCAGTC
TCCAAAGCTCCTGATCTACAAAGTTTCCAACCGATTTTCTGGGGTCCCAG
ACAGGTTCAGTGGCAGTGGATCAGGGACAGATTTCACACTCAAGATCAGC
AGAGTGGAGGCTGAGGATCTGGGAGTTTATTTCTGCTCTCACAGTACACA
TGTTCCATTCACGTTCGGCTCGGGGACAAAGTTGGAAATAAAAcgggctg
atgctgcaccaactgtatccatcttcccaccatccagtgagcagttaaca
tctggag

The amino acid sequence of the light chain variable region of the mouse 8C11 antibody (152 a. a.) [capital letters: mouse 8C11 VL variable region, small letters: mouse Ig [symbol] light chain constant region] The underlined sequence shows its signal sequence and the double underline shows its CDR regions (CDR1, CDR2 and CDR3).

MKLPVRLLVLMFWIPASSSDVVMTQTPLSLPVSLGDQASISCTSSQTLVH
SNGNTYLHWYLQKPGQSPKLLIYKVSNRFSGVPDRFSGSGSGTDFTLKIS
RVEAEDLGVYFCSHSTHVPFTFGSGTKLEIKradaaptvsifppsseqlt
sg

CDR1 in the light chain variable region of the 8C11 antibody

### TSSQTLVHSNGNTYLH

CDR2 in the light chain variable region of the 8C11 antibody

### KVSNRFS

CDR3 in the light chain variable region of the 8C11 antibody

### HSTHVP

### 6. Anti-PLD4 Mouse 10C3 Antibody

The nucleic acid sequence of the heavy chain variable region of the obtained anti-PLD4 mouse 10C3 antibody is SEQ ID NO: 84, and the amino acid sequence is SEQ ID NO: 85. The amino acid sequences of CDR1, CDR2 and CDR3 within the heavy chain variable region of the mouse 10C3 antibody are SEQ ID NO: 26, SEQ ID NO: 27 and SEQ ID NO: 28, respectively.

The nucleic acid sequence of the heavy chain variable region of the anti-PLD4 mouse 10C3 antibody (450 bp) [capital letters: mouse 10C3 VH variable region, small letters: mouse IgG2a heavy chain constant region]

ATGAACTTCGGGCTCAGCTTGATTTTCCTTGCCCTCATTTTAAAAGGTGT
CCAGTGTGAGGTGCAGCTGGTGGAGTCTGGGGGAGACTTAGTGAGGCCTG
GAGGGTCCCTGAAACTCTCCTGTGCAGCCTCTGGATTCAGTTTCAGTAGC
TATGGCATGTCTTGGTTTCGCCAGACTCCAGACAAGAGGCTGGAGTGGGT
CGCAACCATTAGTAGTGGTGGTAGTTACATCTACTATCCAGAAAGTGTGA
AGGGGCGATTCACCATCTCCAGAGACAATGCCAGGAACATCCTGTACCTG
CAAATGAGCAGTCTGAAGTCTGAGGACACAGCCATGTATTATTGTGTAAG
ACTCTACGGTGGTAGGAGAGGCTATGGTTTGGACTACTGGGGTCAAGGAA
CCTCAGTCACCGTCTCCTCAgccaaaacaacagccccatcggtctatcca

The amino acid sequence of the heavy chain variable region of the mouse 10C3 antibody (150 a. a.) [capital letters: mouse 10C3 VH variable region, small letters: mouse IgG2a heavy chain constant region] The underlined sequence shows its signal sequence and the double underline shows its CDR regions (CDR1, CDR2 and CDR3).

MNFGLSLIFLALILKGVQCEVQLVESGGDLVRPGGSLKLSCAASGFSFSS
YGMSWFRQTPDKRLEWVATISSGGSYIYYPESVKGRFTISRDNARNILYL
QMSSLKSEDTAMYYCVRLYGGRRGYGLDYWGQGTSVTVSSakttapsvyp

CDR1 in the heavy chain variable region of the 10C3 antibody

### SYGMS

CDR2 in the heavy chain variable region of the 10C3 antibody

### TISSGGSYIYYPESVKG

CDR3 in the heavy chain variable region of the 10C3 antibody

### LYGGRRGYGLDY

The nucleic acid sequence of the light chain variable region of the obtained anti-PLD4 mouse 10C3 antibody is SEQ ID NO: 104, and the amino acid sequence is SEQ ID NO: 105. The amino acid sequences of CDR1, CDR2 and CDR3 within the light chain variable region of the mouse 10C3 antibody are SEQ ID NO: 29, SEQ ID NO: 30 and SEQ ID NO: 31, respectively.

The nucleic acid sequence of the light chain variable region of the anti-PLD4 mouse 10C3 antibody (423 bp) [capital letters: mouse 10C3 VL variable region, small letters: mouse Ig [symbol] light chain constant region]

ATGAGGTTCTCTGCTCAGCTTCTGGGGCTGCTTGTGCTCTGGATCCCTGG
ATCCACTGCGGAAATTGTGATGACGCAGGCTGCATTCTCCAATCCAGTCA
CTCTTGGAACATCAGCTTCCATCTCCTGCAGGTCTAGTAAGAGTCTCCTA
CATAGTGATGGCATCACTTATTTGTATTGGTATCTGCAGAAGCCAGGCCA
GTCTCCTCAGCTCCTGATTTATCAGATGTCCAACCTTGCCTCAGGAGTCC
CAGACAGGTTCAGTAGCAGTGGGTCAGGAACTGATTTCACACTGAGAATC
AGCAGAGTGGAGGCTGAGGATGTGGGTGTTTATTACTGTGCTCAAAATCT
AGAACTTTACACGTTCGGAGGGGGGACCAAGCTGGAAATAAAAcgggctg
atgctgcaccaactgtatccatc

The amino acid sequence of the light chain variable region of the mouse 10C3 antibody (141 a. a.) [capital letters: mouse 10C3 VL variable region, small letters: mouse Ig [symbol] light chain constant region] The underlined sequence shows its signal sequence and the double underline shows its CDR regions (CDR1, CDR2 and CDR3).

MRFSAQLLGLLVLWIPGSTAEIVMTQAAFSNPVTLGTSASISCRSSKSLL
HSDGITYLYWYLQKPGQSPQLLIYQMSNLASGVPDRFSSSGSGTDFTLRI
SRVEAEDVGVYYCAQNLELYTFGGGTKLEIKradaaptvsi

CDR1 in the light chain variable region of the 10C3 antibody

### RSSKSLLHSDGITYLY

CDR2 in the light chain variable region of the 10C3 antibody

### QMSNLAS

CDR3 in the light chain variable region of the 10C3 antibody

### AQNLEL

### 7. Anti-PLD4 Mouse 11D10 Antibody

The nucleic acid sequence of the heavy chain variable region of the obtained anti-PLD4 mouse 11D10 antibody is SEQ ID NO: 86, and the amino acid sequence is SEQ ID NO: 87. The amino acid sequences of CDR1, CDR2 and CDR3 within the heavy chain variable region of the mouse 11D10 antibody are SEQ ID NO: 26, SEQ ID NO: 27 and SEQ ID NO: 28, respectively.

The nucleic acid sequence of the heavy chain variable region of the anti-PLD4 mouse 11D10 antibody (450 bp) [capital letters: mouse 11D10 VH variable region, small letters: mouse IgG2b heavy chain constant region]

ATGAACTTCGGGCTCAGCTTGATTTTCCTTGCCCTCATTTTAAAAGGTGT
CCAGTGTGAGGTGCAGCTGGTGGAGTCTGGGGGAGACTTAGTGAGGCCTG
GAGGGTCCCTGAAACTCTCCTGTGCAGCCTCTGGATTCAGTTTCAGTAGC
TATGGCATGTCTTGGTTTCGCCAGACTCCAGACAAGAGGCTGGAGTGGGT
CGCAACCATTAGTAGTGGTGGTAGTTACATCTACTATCCAGAAAGTGTGA
AGGGGCGATTCACCATCTCCAGAGACAATGCCAGGAACATCCTGTACCTG
CAAATGAGCAGTCTGAAGTCTGAGGACACAGCCATGTATTATTGTGTAAG
ACTCTACGGTGGTAGGAGAGGCTATGGTTTGGACTACTGGGGTCAAGGAA
CCTCAGTCACCGTCTCCTCAgccaaaacaacacccccatcagtctatcca

The amino acid sequence of the heavy chain variable region of the mouse 11D10 antibody (150 a. a.) [capital letters: mouse 11D10 VH variable region, small letters: mouse IgG2b heavy chain constant region] The underlined sequence shows its signal sequence and the double underline shows its CDR regions (CDR1, CDR2 and CDR3).

MNFGLSLIFLALILKGVQCEVQLVESGGDLVRPGGSLKLSCAASGFSFSS
YGMSWFRQTPDKRLEWVATISSGGSYIYYPESVKGRFTISRDNARNILYL
QMSSLKSEDTAMYYCVRLYGGRRGYGLDYWGQGTSVTVSS akttppsvy
p

CDR1 in the heavy chain variable region of the 11D10 antibody

### SYGMS

CDR2 in the heavy chain variable region of the 11D10 antibody

### TISSGGSYIYYPESVKG

CDR3 in the heavy chain variable region of the 11D10 antibody

### LYGGRRGYGLDY

The nucleic acid sequence of the light chain variable region of the obtained anti-PLD4 mouse 11D10 antibody is SEQ ID NO: 106, and the amino acid sequence is SEQ ID NO: 107. The amino acid sequences of CDR1, CDR2 and CDR3 within the light chain variable region of the mouse 11D10 antibody are SEQ ID NO: 29, SEQ ID NO: 30 and SEQ ID NO: 31, respectively.

The nucleic acid sequence of the light chain variable region of the anti-PLD4 mouse 11D10 antibody (423 bp) [capital letters: mouse 11D10 VL variable region, small letters: mouse Ig [symbol] light chain constant region]

ATGAGGTTCTCTGCTCAGCTTCTGGGGCTGCTTGTGCTCTGGATCCCTGG
ATCCACTGCGGAAATTGTGATGACGCAGGCTGCATTCTCCAATCCAGTCA
CTCTTGGAACATCAGCTTCCATCTCCTGCAGGTCTAGTAAGAGTCTCCTA
CATAGTGATGGCATCACTTATTTGTATTGGTATCTGCAGAAGCCAGGCCA
GTCTCCTCAGCTCCTGATTTATCAGATGTCCAACCTTGCCTCAGGAGTCC
CAGACAGGTTCAGTAGCAGTGGGTCAGGAACTGATTTCACACTGAGAATC
AGCAGAGTGGAGGCTGAGGATGTGGGTGTTTATTACTGTGCTCAAAATCT
AGAACTTTACACGTTCGGAGGGGGGACCAAGCTGGAAATAAAAcgggctg
atgctgcaccaactgtatccatc

The amino acid sequence of the light chain variable region of the mouse 11D10 antibody (141 a. a.) [capital letters: mouse 11D10 VL variable region, small letters: mouse Ig [symbol] light chain constant region] The underlined sequence shows its signal sequence and the double underline shows its CDR regions (CDR1, CDR2 and CDR3).

MRFSAQLLGLLVLWIPGSTAEIVMTQAAFSNPVTLGTSASISCRSSKSLL
HSDGITYLYWYLQKPGQSPQLLIYQMSNLASGVPDRFSSSGSGTDFTLRI
SRVEAEDVGVYYCAQNLELYTFGGGTKLEIKradaaptvsi

CDR1 in the light chain variable region of the 11D10 antibody

### RSSKSLLHSDGITYLY

CDR2 in the light chain variable region of the 11D10 antibody

### QMSNLAS

CDR3 in the light chain variable region of the 11D10 antibody

### AQNLEL

### 8. Anti-PLD4 Mouse 13D4 Antibody

The nucleic acid sequence of the heavy chain variable region of the obtained anti-PLD4 mouse 13D4 antibody is SEQ ID NO: 88, and the amino acid sequence is SEQ ID NO: 89. The amino acid sequences of CDR1, CDR2 and CDR3 within the heavy chain variable region of the mouse 13D4 antibody are SEQ ID NO: 32, SEQ ID NO: 33 and SEQ ID NO: 34, respectively.

The nucleic acid sequence of the heavy chain variable region of the anti-PLD4 mouse 13D4 antibody (472 bp) [capital letters: mouse 13D4 VH variable region, small letters: mouse IgG2b heavy chain constant region]

ATGAAAGTGTTGAGTCTGTTGTACCTGTTGACAGCCATTCCTGGTATCCT
GTCTGATGTACAGCTTCAGGAGTCAGGACCTGGCCTCGTGAAACCTTCTC
AATCTCTGTCTCTCACCTGCTCTGTCACTGGCTACTCCATCACCAGTCAT
TATTACTGGACCTGGATCCGGCAGTTTCCAGGAAACAAACTGGAATGGAT
GGGCTACATAAGCTACGACGGTAGCAATAACTACAACCCATCTCTCAAAA
ATCGAATCTCCATCACTCGTGACACATCTAAGAACCAGTTTTTCCTGAAG
TTGAATTCTGTGACTACTGAGGACACAGCTACATATAACTGTGCAAGAGA
GGGCCCGCTCTACTATGGTAACCCCTACTGGTATTTCGATGTCTGGGGCG
CAGGGACCACGGTCACCGTCTCCTCAgccaaaacaacacccccatcagtc
tatccactggcccctaagggcg

The amino acid sequence of the heavy chain variable region of the mouse 13D4 antibody (157 a. a.) [capital letters: mouse 13D4 VH variable region, small letters: mouse IgG2b heavy chain constant region] The underlined sequence shows its signal sequence and the double underline shows its CDR regions (CDR1, CDR2 and CDR3).

MKVLSLLYLLTAIPGILSDVQLQESGPGLVKPSQSLSLTCSVTGYSITSH
YYWTWIRQFPGNKLEWMGYISYDGSNNYNPSLKNRISITRDTSKNQFFLK
LNSVTTEDTATYNCAREGPLYYGNPYWYFDVWGAGTTVTVSSakttppsv
yplapkg

CDR1 in the heavy chain variable region of the 13D4 antibody

### SHYYWT

CDR2 in the heavy chain variable region of the 13D4 antibody

### YISYDGSNNYNPSLKN

CDR3 in the heavy chain variable region of the 13D4 antibody

### EGPLYYGNPYWYFDV

The nucleic acid sequence of the light chain variable region of the obtained anti-PLD4 mouse 13D4 antibody is SEQ ID NO: 108, and the amino acid sequence is SEQ ID NO: 109. The amino acid sequences of CDR1, CDR2 and CDR3 within the light chain variable region of the mouse 13D4 antibody are SEQ ID NO: 35, SEQ ID NO: 36 and SEQ ID NO: 37, respectively.

The nucleic acid sequence of the light chain variable region of the anti-PLD4 mouse 13D4 antibody (404 bp) [capital letters: mouse 13D4 VL variable region, small letters: mouse Ig [symbol] light chain constant region]

ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGGGACAGAGTCACCATCAGTTGCAGGGCAAGTCAGGACATTGAC
AATTATTTAAACTGGTATCAGCAGAAACCAGATGGAACTGTTAAACTCCT
GATCTACTACACATCAAGATTACACTCAGGAGTCCCATCAAGGTTCAGTG
GCAGTGGGTCTGGAACAGATTATTCTCTCACCATTAGCAACCTGGAGCAA
GAAGATGTTGCCACTTACTTTTGCCAGCAGTTTAATACGCTTCCTCGGAC
GTTCGGTGGAGGCACCAAACTGGAAATCAAAcgggctgatgctgcaccaa
ctgt

The amino acid sequence of the light chain variable region of the mouse 13D4 antibody (134 a. a.) [capital letters: mouse 13D4 VL variable region, small letters: mouse Ig [symbol] light chain constant region] The underlined sequence shows its signal sequence and the double underline shows its CDR regions (CDR1, CDR2 and CDR3).

MMSSAQFLGLLLLCFQGTRCDIQMTQTTSSLSASLGDRVTISCRASQDID
NYLNWYQQKPDGTVKLLIYYTSRLHSGVPSRFSGSGSGTDYSLTISNLEQ
EDVATYFCQQFNTLPRTFGGGTKLEIKradaapt

CDR1 in the light chain variable region of the 13D4 antibody

### RASQDIDNYLN

CDR2 in the light chain variable region of the 13D4 antibody

### YTSRLHS

CDR3 in the light chain variable region of the 13D4 antibody

### QQFNTLP

### 9. Anti-PLD4 Mouse 13H11 Antibody

The nucleic acid sequence of the heavy chain variable region of the obtained anti-PLD4 mouse 13H11 antibody is SEQ ID NO: 90, and the amino acid sequence is SEQ ID NO: 91. The amino acid sequences of CDR1, CDR2 and CDR3 within the heavy chain variable region of the mouse 13H11 antibody are SEQ ID NO: 38, SEQ ID NO: 39 and SEQ ID NO: 40, respectively.

The nucleic acid sequence of the heavy chain variable region of the anti-PLD4 mouse 13H11 antibody (471 bp) [capital letters: mouse 13H11 VH variable region, small letters: mouse IgG2b heavy chain constant region]

ATGAAAGTGTTGAGTCTGTTGTACCTGTTGACAGCCATTCCTGGTATCCT
GTCTGATGTACAGCTTCAGGAGTCAGGACCTGGCCTCGTGAAACCTTCTC
AGTCTCTGTCTCTCACCTGCTCTGTCACTGGCTACTCCATCTCCAGTCAT
TATTACTGGAGTTGGATCCGGCAGTTTCCAGGAAACAGACTGGAATGGAT
GGGCTACATAAGCTACGACGGTAGCAATAACTACAACCCATCTCTCAAAA
ATCGAATCTCCATCACTCGTGACACATCTAAGAACCAGTTTTTCCTGAAG
TTGAATTCTGTGACTACTGAGGACACAGCTACATATAACTGTGCAAGAGA
GGGCCCGCTCTACTATGGTAACCCCTACTGGTATTTCGATGTCTGGGGCG
CAGGGACCACGGTCACCGTCTCCTCAgccaaaacaacacccccatcagtc
tatccactggcccctaagggc

The amino acid sequence of the heavy chain variable region of the mouse 13H11 antibody (157 a. a.) [capital letters: mouse 13H11 VH variable region, small letters: mouse IgG2b heavy chain constant region] The underlined sequence shows its signal sequence and the double underline shows its CDR regions (CDR1, CDR2 and CDR3).

MKVLSLLYLLTAIPGILSDVQLQESGPGLVKPSQSLSLTCSVTGYSISSH
YYWSWIRQFPGNRLEWMGYISYDGSNNYNPSLKNRISITRDTSKNQFFLK
LNSVTTEDTATYNCAREGPLYYGNPYWYFDVWGAGTTVTVSSakttppsv
yplapkg

CDR1 in the heavy chain variable region of the 13H11 antibody

### SHYYWS

CDR2 in the heavy chain variable region of the 13H11 antibody

### YISYDGSNNYNPSLKN

CDR3 in the heavy chain variable region of the 13H11 antibody

### EGPLYYGNPYWYFDV

The nucleic acid sequence of the light chain variable region of the obtained anti-PLD4 mouse 13H11 antibody is SEQ ID NO: 110, and the amino acid sequence is SEQ ID NO: 111. The amino acid sequences of CDR1, CDR2 and CDR3 within the light chain variable region of the mouse 13H11 antibody are SEQ ID NO: 41, SEQ ID NO: 42 and SEQ ID NO: 43, respectively.

The nucleic acid sequence of the light chain variable region of the anti-PLD4 mouse 13H11 antibody (414 bp) [capital letters: mouse 13H11 VL variable region, small letters: mouse Ig [symbol] light chain constant region]

ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGGGGCAGCGTCACCATCAGTTGCAGGGCAAGTCAGGACATTGAC
AATTATTTAAACTGGTATCAGCAAAAACCAGATGGAACTGTTAAACTCCT
GATCTACTACACATCAAGATTACACTCAGGAGTCCCATCAAGGTTCAGTG
GCAGTGGGTCTGGAACAGATTATTCTCTCACCATTAGCAACCTGGAACAA
GAAGATATTGCCACTTACTTTTGCCAACAGTTTAATACGCTTCCTCGGAC
GTTCGGTGGAGGCACCAAGCTGGAAATCAAAcgggctgatgctgcaccaa
ctgtatccatcttc

The amino acid sequence of the light chain variable region of the mouse 13H11 antibody (138 a. a.) [capital letters: mouse 13H11 VL variable region, small letters: mouse Ig [symbol] light chain constant region] The underlined sequence shows its signal sequence and the double underline shows its CDR regions (CDR1, CDR2 and CDR3).

MMSSAQFLGLLLLCFQGTRCDIQMTQTTSSLSASLGGSVTISCRASQDID
NYLNWYQQKPDGTVKLLIYYTSRLHSGVPSRFSGSGSGTDYSLTISNLEQ
EDIATYFCQQFNTLPRTFGGGTKLEIKradaaptvsif

CDR1 in the light chain variable region of the 13H11 antibody

### RASQDIDNYLN

CDR2 in the light chain variable region of the 13H11 antibody

### YTSRLHS

CDR3 in the light chain variable region of the 13H11 antibody

### QQFNTLP

### 10. Anti-PLD4 Mouse 14C1 Antibody

The nucleic acid sequence of the heavy chain variable region of the obtained anti-PLD4 mouse 14C1 antibody is SEQ ID NO: 92, and the amino acid sequence is SEQ ID NO: 93. The amino acid sequences of CDR1, CDR2 and CDR3 within the heavy chain variable region of the mouse 14C1 antibody are SEQ ID NO: 38, SEQ ID NO: 39 and SEQ ID NO: 40, respectively.

The nucleic acid sequence of the heavy chain variable region of the anti-PLD4 mouse 14C1 antibody (470 bp) [capital letters: mouse 14C1 VH variable region, small letters: mouse IgG1 heavy chain constant region]

ATGAAAGTGTTGAGTCTGTTGTACCTGTTGACAGCCATTCCTGGTATCCT
GTCTGATGTACAGCTTCAGGAGTCAGGACCTGGCCTCGTGAAACCTTCTC
AGTCTCTGTCTCTCACCTGCTCTGTCACTGGCTACTCCATCTCCAGTCAT
TATTACTGGAGTTGGATCCGGCAGTTTCCAGGAAACAGACTGGAATGGAT
GGGCTACATAAGCTACGACGGTAGCAATAACTACAACCCATCTCTCAAAA
ATCGAATCTCCATCACTCGTGACACATCTAAGAACCAGTTTTTCCTGAAG
TTGAATTCTGTGACTACTGAGGACACAGCTACATATAACTGTGCAAGAGA
GGGCCCGCTCTACTATGGTAACCCCTACTGGTATTTCGATGTCTGGGGCG
CAGGGACCACGGTCACCGTCTCCTCAgccaaaacgacacccccatctgtc
tatccactggcccctaaggg

The amino acid sequence of the heavy chain variable region of the mouse 14C1 antibody (156 a. a.) [capital letters: mouse 14C1 VH variable region, small letters: mouse IgG1 heavy chain constant region] The underlined sequence shows its signal sequence and the double underline shows its CDR regions (CDR1, CDR2 and CDR3).

MKVLSLLYLLTAIPGILSDVQLQESGPGLVKPSQSLSLTCSVTGYSISSH
YYWSWIRQFPGNRLEWMGYISYDGSNNYNPSLKNRISITRDTSKNQFFLK
INSVTTEDTATYNCAREGPLYYGNPYWYFDVWGAGTTVTVSS
akttppsvyplapk

CDR1 in the heavy chain variable region of the 14C1 antibody

### SHYYWS

CDR2 in the heavy chain variable region of the 14C1 antibody

### YISYDGSNNYNPSLKN

CDR3 in the heavy chain variable region of the 14C1 antibody

### EGPLYYGNPYWYFDV

The nucleic acid sequence of the light chain variable region of the obtained anti-PLD4 mouse 14C1 antibody is SEQ ID NO: 112, and the amino acid sequence is SEQ ID NO: 113. The amino acid sequences of CDR1, CDR2 and CDR3 within the light chain variable region of the mouse 14C1 antibody are SEQ ID NO: 41, SEQ ID NO: 42 and SEQ ID NO: 43, respectively.

The nucleic acid sequence of the light chain variable region of the anti-PLD4 mouse 14C1 antibody (465 bp) [capital letters: mouse 14C1 VL variable region, small letters: mouse Ig [symbol] light chain constant region]

ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGGGGCAGCGTCACCATCAGTTGCAGGGCAAGTCAGGACATTGAC
AATTATTTAAACTGGTATCAGCAAAAACCAGATGGAACTGTTAAACTCCT
GATCTACTACACATCAAGATTACACTCAGGAGTCCCATCAAGGTTCAGTG
GCAGTGGGTCTGGAACAGATTATTCTCTCACCATTAGCAACCTGGAACAA
GAAGATATTGCCACTTACTTTTGCCAACAGTTTAATACGCTTCCTCGGAC
GTTCGGTGGAGGCACCAAGCTGGAAATCAAAcgggctgatgctgcaccaa
ctgtatccatcttcccaccatccagtgagcagttaacatctggaggtgcc
tcagtcgtgtgcttc

The amino acid sequence of the light chain variable region of the mouse 14C1 antibody (155 a. a.) [capital letters: mouse 14C1 VL variable region, small letters: mouse Ig [symbol] light chain constant region] The underlined sequence shows its signal sequence and the double underline shows its CDR regions (CDR1, CDR2 and CDR3).

MMSSAQFLGLLLLCFQGTRCDIQMTQTTSSLSASLGGSVTISCRASQDID
NYLNWYQQKPDGTVKLLIYYTSRLHSGVPSRFSGSGSGTDYSLTISNLEQ
EDIATYFCQQFNTLPRTFGGGTKLEIKradaaptvsifppsseqltsgga
swcf

CDR1 in the light chain variable region of the 14C1 antibody

### RASQDIDNYLN

CDR2 in the light chain variable region of the 14C1 antibody

### YTSRLHS

CDR3 in the light chain variable region of the 14C1 antibody

### QQFNTLP

The base sequences and the amino acid sequences of the heavy chain and the light chain of the created chimeric 11G9.6 antibody are as the sequence numbers given below.

### Heavy Chain

SEQ ID NO: 120 (base sequence)
SEQ ID NO: 121 (amino acid sequence)

### Light Chain

SEQ ID NO: 122 (base sequence)
SEQ ID NO: 123 (amino acid sequence)
11. The nucleic acid sequence of the heavy chain of the anti-PLD4 chimeric 11G9.6 antibody (1401 bp) [capital letters: chimeric 11G9 VH variable region, small letters: human IgG1 heavy chain constant region] (SEQ ID NO: 120)

ATGAAAGTGTTGAGTCTGTTGTACCTGTTGACAGCCATTCCTGGTATCC
TGTCTcagGTCCAACTGCAGCAGCCTGGGGCTGAACTGGTGAAGCCTGG
GACTTCAGTGAAAATGTCCTGCAAGGCTTCTGGCTACACCTTCACCAGC
TACTGGATGCACTGGGTGAAGCAGAGGCCGGGACAAGGCCTTGAGTGGA
TTGGAGATATTTATCCTGGTAGTGATAGTACTAACTACAATGAGAAGTT
CAAGAGCAAGGCCACACTGACTGTAGACACATCCTCCAGCACAGCCTAC
ATGCAACTCAGCAGCCTGACATCTGAGGACTCTGCGGTCTATTACTGTG
CAAGAGGAGGGTGGTTGGATGCTATGGACTACTGGGGTCAAGGAACCTC
AGTCACCGTCTCCTCAgctagcaccaagggcccatcggtcttccccctg
gcaccctcctccaagagcacctctgggggcacagcggccctgggctgcc
tggtcaaggactacttccccgaaccggtgacggtgtcgtggaactcagg
cgccctgaccagcggcgtgcacaccttcccggctgtcctacagtcctca
ggactctactccctcagcagcgtggtgaccgtgccctccagcagcttgg
gcacccagacctacatctgcaacgtgaatcacaagcccagcaacaccaa
ggtggacaagaaagttgagcccaaatcttgtgacaaaactcacacatgc
ccaccgtgcccagcacctgaactcctggggggaccgtcagtcttcctct
tccccccaaaacccaaggacaccctcatgatctcccggacccctgaggt
cacatgcgtggtggtggacgtgagccacgaagaccctgaggtcaagttc
aactggtacgtggacggcgtggaggtgcataatgccaagacaaagccgc
gggaggagcagtacaacagcacgtaccgtgtggtcagcgtcctcaccgt
cctgcaccaggactggctgaatggcaaggagtacaagtgcaaggtctcc
aacaaagccctcccagcccccatcgagaaaaccatctccaaagccaaag
ggcagccccgagaaccacaggtgtacaccctgcccccatcccgggatga
gctgaccaagaaccaggtcagcctgacctgcctggtcaaaggcttctat
cccagcgacatcgccgtggagtgggagagcaatgggcagccggagaaca
actacaagaccacgcctcccgtgctggactccgacggctccttcttcct
ctacagcaagctcaccgtggacaagagcaggtggcagcaggggaacgtc
ttctcatgctccgtgatgcatgaggctctgcacaaccactacacgcaga
agagcctctccctgtctccgggtaaatga

12. The amino acid sequence of the heavy chain of the anti-PLD4 chimeric 11G9.6 antibody (466 a. a.) [capital letters: chimeric 11G9 VH variable region, small letters: human IgG1 heavy chain constant region] (SEQ ID NO: 121)

MKVLSLLYLLTAIPGILSQVQLQQPGAELVKPGTSVKMSCKASGYTFTSY
WMHWVKQRPGQGLEWIGDIYPGSDSTNYNEKFKSKATLTVDTSSSTAYMQ
LSSLTSEDSAVYYCARGGWLDAMDYWGQGTSVTVSSastkgpsvfplaps
skstsggtaalgclykdyfpepvtvswnsgaltsgvhtfpavlqssglys
lssvvtvpssslgtqtyicnvnhkpsntkvdkkvepkscdkthtcppcpa
pellggpsvflfppkpkdtlmisrtpevtcvvvdvshedpevkfnwyvdg
vevhnaktkpreeqynstyrvvsyltvlhqdwlngkeykckvsnkalpap
iektiskakgqprepqvytlppsrdeltknqvsltclvkgfypsdiavew
esngqpennykttppvldsdgsfflyskltvdksrwqqgnvfscsvmhea
lhnhytqkslslspgk

13. The nucleic acid sequence of the light chain of the anti-PLD4 chimeric 11G9.6 antibody (705 bp) [capital letters: chimeric 11G9 VL variable region, small letters: human Ig [symbol] light chain constant region] (SEQ ID NO: 122)

ATGATGTCCTCTGCTCAGTTCCTTGGTCTCCTGTTGCTCTGTTTTCAAGG
TACCAGATGTGATATCCAGATGACACAGACTACATCCTCCCTGTCTGCCT
CTCTGGGAGACAGAGTCACCATCAGTTGCAGGGCAAGTCAGGACATTAGC
AATTATTTAAACTGGTATCAGCAGAAACCAGATGGAACTGTTAAACTCCT
GATCTACTACACATCAAGATTACACTCAGGAGTCCCATCAAGGTTCAGTG
GCAGTGGGTCTGGAACAGATTATTCTCTCACCATTAGCAACCTGGAGCAA
GAAGATATTGCCACTTACTTTTGCCAACAGGGTAATACGCTTCCGTGGAC
GTTCGGTGGAGGCACCAAGCTGGAAATCAAAcgaactgtggctgcaccat
ctgtcttcatcttcccgccatctgatgagcagttgaaatctggaactgcc
tctgttgtgtgcctgctgaataacttctatcccagagaggccaaagtaca
gtggaaggtggataacgccctccaatcgggtaactcccaggagagtgtca
cagagcaggacagcaaggacagcacctacagcctcagcagcaccctgacg
ctgagcaaagcagactacgagaaacacaaagtctacgcctgcgaagtcac
ccatcagggcctgagctcgcccgtcacaaagagcttcaacaggggagagt
gctag

14. The amino acid sequence of the light chain of the anti-PLD4 chimeric 11G9.6 antibody (234 a. a.) [capital letters: chimeric 11G9 VL variable region, small letters: human Ig [symbol] light chain constant region] (SEQ ID NO: 123)

MMSSAQFLGLLLLCFQGTRCDIQMTQTTSSLSASLGDRVTISCRASQDIS
NYLNWYQQKPDGTVKLLIYYTSRLHSGVPSRFSGSGSGTDYSLTISNLEQ
EDIATYFCQQGNTLPWTFGGGTKLEIKrtvaapsvfifppsdeqlksgta
svvcllnnfypreakvqwkvdnalqsgnsqesvteqdskdstyslsstlt
lskadyekhkvyacevthqglsspvtksfnrgec

<cDNA and Protein Sequences of PLD4-Related Molecules>

> Human PLD4 cDNA (1521 bp)
(SEQ ID NO: 44)
ATGCTGAAGCCTCTTTGGAAAGCAGCAGTGGCCCCCACATGGCCATGCTCCATGCCGCCCCGCCGCCCGTGG
GACAGAGAGGCTGGCACGTTGCAGGTCCTGGGAGCGCTGGCTGTGCTGTGGCTGGGCTCCGTGGCTCTTATC
TGCCTCCTGTGGCAAGTGCCCCGTCCTCCCACCTGGGGCCAGGTGCAGCCCAAGGACGTGCCCAGGTCCTGG
GAGCATGGCTCCAGCCCAGCTTGGGAGCCCCTGGAAGCAGAGGCCAGGCAGCAGAGGGACTCCTGCCAGCTT
GTCCTTGTGGAAAGCATCCCCCAGGACCTGCCATCTGCAGCCGGCAGCCCCTCTGCCCAGCCTCTGGGCCAG
GCCTGGCTGCAGCTGCTGGACACTGCCCAGGAGAGCGTCCACGTGGCTTCATACTACTGGTCCCTCACAGGG
CCTGACATCGGGGTCAACGACTCGTCTTCCCAGCTGGGAGAGGCTCTTCTGCAGAAGCTGCAGCAGCTGCTG
GGCAGGAACATTTCCCTGGCTGTGGCCACCAGCAGCCCGACACTGGCCAGGACATCCACCGACCTGCAGGTT
CTGGCTGCCCGAGGTGCCCATGTACGACAGGTGCCCATGGGGCGGCTCACCAGGGGTGTTTTGCACTCCAAA
TTCTGGGTTGTGGATGGACGGCACATATACATGGGCAGTGCCAACATGGACTGGCGGTCTCTGACGCAGGTG
AAGGAGCTTGGCGCTGTCATCTATAACTGCAGCCACCTGGCCCAAGACCTGGAGAAGACCTTCCAGACCTAC
TGGGTACTGGGGGTGCCCAAGGCTGTCCTCCCCAAAACCTGGCCTCAGAACTTCTCATCTCACTTCAACCGT
TTCCAGCCCTTCCACGGCCTCTTTGATGGGGTGCCCACCACTGCCTACTTCTCAGCGTCGCCACCAGCACTC
TGTCCCCAGGGCCGCACCCGGGACCTGGAGGCGCTGCTGGCGGTGATGGGGAGCGCCCAGGAGTTCATCTAT
GCCTCCGTGATGGAGTATTTCCCCACCACGCGCTTCAGCCACCCCCCGAGGTACTGGCCGGTGCTGGACAAC
GCGCTGCGGGCGGCAGCCTTCGGCAAGGGCGTGCGCGTGCGCCTGCTGGTCGGCTGCGGACTCAACACGGAC
CCCACCATGTTCCCCTACCTGCGGTCCCTGCAGGCGCTCAGCAACCCCGCGGCCAACGTCTCTGTGGACGTG
AAAGTCTTCATCGTGCCGGTGGGGAACCATTCCAACATCCCATTCAGCAGGGTGAACCACAGCAAGTTCATG
GTCACGGAGAAGGCAGCCTACATAGGCACCTCCAACTGGTCGGAGGATTACTTCAGCAGCACGGCGGGGGTG
GGCTTGGTGGTCACCCAGAGCCCTGGCGCGCAGCCCGCGGGGGCCACGGTGCAGGAGCAGCTGCGGCAGCTC
TTTGAGCGGGACTGGAGTTCGCGCTACGCCGTCGGCCTGGACGGACAGGCTCCGGGCCAGGACTGCGTTTGG
CAGGGCTGA
> Human PLD4 protein (506 amino acids)
(SEQ ID NO: 1)
MLKPLWKAAVAPTWPCSMPPRRPWDREAGTLQVLGALAVLWLGSVALICLLWQVPRPPTWGQVQPKDVPRSW
EHGSSPAWEPLEAEARQQRDSCQLVLVESIPQDLPSAAGSPSAQPLGQAWLQLLDTAQESVHVASYYWSLTG
PDIGVNDSSSQLGEALLQKLQQLLGRNISLAVATSSPTLARTSTDLQVLAARGAHVRQVPMGRLTRGVLHSK
FWVVDGRHIYMGSANMDWRSLTQVKELGAVIYNCSHLAQDLEKTFQTYWVLGVPKAVLPKTWPQNFSSHFNR
FQPFHGLFDGVPTTAYFSASPPALCPQGRTRDLEALLAVMGSAQEFIYASVMEYFPTTRFSHPPRYWPVLDN
ALRAAAFGKGVRVRLLVGCGLNTDPTMFPYLRSLQALSNPAANVSVDVKVFIVPVGNHSNIPFSRVNHSKFM
VTEKAAYIGTSNWSEDYFSSTAGVGLVVTQSPGAQPAGATVQEQLRQLFERDWSSRYAVGLDGQAPGQDCVW
QG
> Cynomolgus monkey PLD4 cDNA (1521 bp)
(SEQ ID NO: 63)
ATGCTGAAGCCTCTTCGGAGAGCgGCAGTGACCCCCATGTGGCCGTGCTCCATGCTGCCCCGCCGCCTGTGG
GACAGAGAGGCTGGCACGTTGCAGGTCCTGGGAGTGCTGGCTATGCTGTGGCTGGGCTCCATGGCTCTTACC
TACCTCCTGTGGCAAGTGCGCCGTCCTCCCACCTGGGGCCAGGTGCAGCCCAAGGACGTGCCCAGGTCCTGG
GGGCATGGTTCCAGCCCAGCTCTGGAGCCCCTGGAAGCGGAGGTCAGGAAGCAGAGGGACTCCTGCCAGCTT
GTCCTTGTGGAAAGCATCCCCCAGGACCTGCCATTTGCAGCCGGCAGCCTCTCCGCCCAGCCTCTGGGCCAG
GCCTGGCTGCAGCTGCTGGACACTGCCCAGGAGAGCGTCCACGTGGCTTCATACTACTGGTCCCTCACAGGG
CCCGACATTGGGGTCAACGACTCATCTTCCCAGCTGGGAGAGGCCCTTCTGCAGAAGCTGCAGCAGCTGCTG
GGCAGGAACATTTCCTTGGCTGTGGCCACCAGCAGTCCAACACTGGCCAGGAAGTCCACCGACCTGCAGGTC
CTGGCTGCCCGAGGTGCCCAGGTACGACGGGTGCCCATGGGGCGGCTCACCAGGGGCGTTTTGCACTCCAAA
TTCTGGGTTGTGGATGGACgGCACATATACATGGGCAGTGCcAACATGGACTGGCGGTCCCTGACGCAGGTG
AAGGAGCTTGGCGCTGTCATCTATAACTGCAGCCACCTGGCCCAAGACCTGGAGAAGACCTTCCAGACCTAC
TGGGTGCTGGGGGTGCCCAAGGCTGTCCTCCCCAAAACCTGGCCTCAGAACTTCTCATCTCACATCAACCGT
TTCCAGCCCTTCCAGGGCCTCTTTGATGGGGTGCCCACCACTGCCTACTTCTCAGCATCGCCACCcGCACTC
TGTCCCCAGGGCCGCACCCCTGACCTGGAGGCGCTGTTGGCGGTGATGGGGAGCGCCCAGGAGTTCATCTAT
GCCTCCGTGATGGAGTATTTCCCTACCACgCGCTTCAGCCACCCCCGCAGGTACTGGCCGGTGCTGGACAAC
GCGCTGCGGGCGGCAGCCTTCAGCAAGGGTGTGCGCGTGCGCCTGCTGGTCAGCTGCGGACTCAACACGGAC
CCCACCATGTTCCCCTATCTGCGGTCCCTGCAGGCGCTCAGCAACCCCGCGGCCAACGTCTCTGTGGACGTG
AAAGTCTTCATCGTGCCGGTGGGGAATCATTCCAACATCCCGTTCAGCAGGGTGAACCACAGCAAGTTCATG
GTCACGGAGAAGGCAGCCTACATAGGCACCTCCAACTGGTCGGAGGATTACTTCAGCAGCACGACGGGGGTG
GGCCTGGTGGTCACCCAGAGCCCCGGCGCGCAGCCCGCGGGGGCCACGGTACAGGAGCAGCTGCGGCAGCTC
TTTGAGCGGGACTGGAGTTCGCGCTACGCCGTCGGCCTGGACGGACAGGCTCCGGGCCAGGACTGCGTTTGG
CAGGGCTGA
> Cynomolgus monkey PLD4 protein (506 amino acids)
(SEQ ID NO: 129)
MLKPLRRAAVTPMWPCSMLPRRLWDREAGTLQVLGVLAMLWLGSMALTYLLWQVRRPPTWGQVQPKDVPRSW
GHGSSPALEPLEAEVRKQRDSCQLVLVESIPQDLPFAAGSLSAQPLGQAWLQLLDTAQESVHVASYYWSLTG
PDIGVNDSSSQLGEALLQKLQQLLGRNISLAVATSSPTLARKSTDLQVLAARGAQVRRVPMGRLTRGVLHSK
FWVVDGRHIYMGSANMDWRSLTQVKELGAVIYNCSHLAQDLEKTFQTYWVLGVPKAVLPKTWPQNFSSHINR
FQPFQGLFDGVPTTAYFSASPPALCPQGRTPDLEALLAVMGSAQEFIYASVMEYFPTTRFSHPRRYWPVLDN
ALRAAAFSKGVRVRLLVSCGLNTDPTMFPYLRSLQALSNPAANVSVDVKVFIVPVGNHSNIPFSRVNHSKFM
VTEKAAYIGTSNWSEDYFSSTTGVGLVVTQSPGAQPAGATVQEQLRQLFERDWSSRYAVGLDGQAPGQDCVW
QG
> Rhesus monkey PLD4 cDNA (1521 bp)
(SEQ ID NO: 124)
ATGCTGAAGCCTCTTCGGAGAGCGGCAGTGACCCCCATGTGGCCGTGCTCCATGCTGCCCCGCCGCCTGTGG
GACAGAGAGGCTGGCACGTTGCAGGTCCTGGGAGTGCTGGCTATGCTGTGGCTGGGCTCCATGGCTCTTACC
TACCTCCTGTGGCAAGTGCGCTGTCCTCCCACCTGGGGCCAGGTGCAGCCCAGGGACGTGCCCAGGTCCTGG
GGGCATGGTTCCAGCCTAGCTCTGGAGCCCCTGGAAGCGGAGGTCAGGAAGCAGAGGGACTCCTGCCAGCTT
GTCCTTGTGGAAAGCATCCCCCAGGACCTGCCATTTGCAGCCGGCAGCCTCTCCGCCCAGCCTCTGGGCCAG
GCCTGGCTGCAGCTGCTGGACACTGCCCAGGAGAGCGTCCACGTGGCTTCATACTACTGGTCCCTCACAGGG
CCCGACATTGGGGTCAACGACTCATCTTCCCAGCTGGGAGAGGCCCTTCTGCAGAAGCTGCAGCAGCTGCTG
GGCAGGAACATTTCCTTGGCTGTGGCCACCAGCAGTCCAACACTGGCCAGGAAGTCCACCGACCTGCAGGTC
CTGGCTGCCCGAGGTGCCCAGGTACGACGGGTGCCCATGGGGCGGCTCACCAGGGGCGTTTTGCACTCCAAA
TTCTGGGTTGTGGATGGACGGCACATATACATGGGCAGTGCCAACATGGACTGGCGGTCCCTGACGCAGGTG
AAGGAGCTTGGCGCTGTCATCTATAACTGCAGCCACCTGGCCCAAGACCTGGAGAAGACCTTCCAGACCTAC
TGGGTGCTGGGGGTGCCCAAGGCTGTCCTCCCCAAAACCTGGCCTCAGAACTTCTCATCTCACATCAACCGT
TTCCAGCCCTTCCAGGGCCTCTTTGATGGGGTGCCCACCACTGCCTACTTCTCAGCATCGCCACCCGCACTC
TGTCCCCAGGGCCGCACCCCTGACCTGGAGGCGCTGTTGGCGGTGATGGGGAGCGCCCAGGAGTTCATCTAT
GCCTCCGTGATGGAGTATTTCCCTACCACGCGCTTCAGCCACCCCCGCAGGTACTGGCCGGTGCTGGACAAC
GCGCTGCGGGCGGCAGCCTTCAGCAAGGGTGTGCGCGTGCGCCTGCTGGTCAGCTGCGGACTCAACACGGAC
CCCACCATGTTCCCCTATCTGCGGTCCCTGCAGGCGCTCAGCAACCCCGCGGCCAACGTCTCTGTGGACGTG
AAAGTCTTCATCGTGCCGGTGGGGAATCATTCCAACATCCCGTTCAGCAGGGTGAACCACAGCAAGTTCATG
GTCACGGAGAAGGCAGCCTACATAGGCACCTCCAACTGGTCGGAGGATTACTTCAGCAGCACGACGGGGGTG
GGCCTGGTGGTCACCCAGAGCCCCGGCGCGCAGCCCGCGGGGGCCACGGTACAGGAGCAGCTGCGGCAGCTC
TTTGAGCGGGACTGGAGTTCGCGCTACGCCGTCGGCCTGGACGGACAGGCTCCGGGCCAGGACTGCGTTTGG
CAGGGCTGA
> Rhesus monkey PLD4 protein (506 amino acids)
(SEQ ID NO: 130)
MLKPLRRAAVTPMWPCSMLPRRLWDREAGTLQVLGVLAMLWLGSMALTYLLWQVRCPPTWGQVQPRDVPRSW
GHGSSLALEPLEAEVRKQRDSCQLVLVESIPQDLPFAAGSLSAQPLGQAWLQLLDTAQESVHVASYYWSLTG
PDIGVNDSSSQLGEALLQKLQQLLGRNISLAVATSSPTLARKSTDLQVLAARGAQVRRVPMGRLTRGVLHSK
FWVVDGRHIYMGSANMDWRSLTQVKELGAVIYNCSHLAQDLEKTFQTYWVLGVPKAVLPKTWPQNFSSHINR
FQPFQGLFDGVPTTAYFSASPPALCPQGRTPDLEALLAVMGSAQEFIYASVMEYFPTTRFSHPRRYWPVLDN
ALRAAAFSKGVRVRLLVSCGLNTDPTMFPYLRSLQALSNPAANVSVDVKVFIVPVGNHSNIPFSRVNHSKFM
VTEKAAYIGTSNWSEDYFSSTTGVGLVVTQSPGAQPAGATVQEQLRQLFERDWSSRYAVGLDGQAPGQDCVW
QG
> Mouse PLD4 cDNA (1512 base pairs)
(SEQ ID NO: 131)
ATGGACAAGAAGAAAGAGCACCCAGAGATGCGGATACCACTCCAGACAGCAGTGGAGGTCTCTGATTGGCCC
TGCTCCACATCTCATGATCCACATAGCGGACTTGGCATGGTACTGGGGATGCTAGCTGTACTGGGACTCAGC
TCTGTGACTCTCATCTTGTTCCTGTGGCAAGGGGCCACTTCTTTCACCAGTCATCGGATGTTCCCTGAGGAA
GTGCCCTCCTGGTCCTGGGAGACCCTGAAAGGAGACGCTGAGCAGCAGAATAACTCCTGTCAGCTCATCCTT
GTGGAAAGCATCCCCGAGGACTTGCCATTTGCAGCTGGCAGCCCCACTGCCCAGCCCCTGGCCCAGGCTTGG
CTGCAGCTTCTTGACACTGCTCGGGAGAGCGTCCACATTGCCTCGTACTACTGGTCCCTCACTGGACTGGAC
ATTGGAGTCAATGACTCGTCTTCTCGGCAGGGAGAGGCCCTTCTACAGAAGTTCCAACAGCTTCTTCTCAGG
AACATCTCTGTGGTGGTGGCCACCCACAGCCCAACATTGGCCAAGACATCCACTGACCTCCAGGTCTTGGCT
GCCCATGGTGCCCAGATACGACAAGTGCCCATGAAACAGCTTACTGGGGGTGTTCTACACTCCAAATTCTGG
GTTGTGGATGGGCGACACGTCTACGTGGGCAGCGCCAACATGGACTGGCGGTCCCTGACTCAGGTGAAGGAA
CTTGGTGCAATCATCTACAACTGCAGCAACCTGGCTCAAGACCTTGAGAAAACATTCCAGACCTACTGGGTG
CTAGGGACTCCCCAAGCTGTTCTCCCTAAAACCTGGCCTCGGAACTTCTCATCCCACATCAACCGCTTCCAT
CCCTTGCGGGGTCCCTTTGATGGGGTTCCCACCACGGCCTATTTCTCGGCCTCCCCTCCCTCCCTCTGCCCG
CATGGCCGGACCCGGGATCTGGACGCAGTGTTGGGAGTGATGGAGGGTGCTCGCCAGTTCATCTATGTCTCG
GTGATGGAGTATTTCCCTACCACGCGCTTCACCCACCATGCCAGGTACTGGCCCGTGCTGGACAATGCGCTA
CGGGCAGCGGCCCTCAATAAGGGTGTGCATGTGCGCTTACTGGTCAGCTGCTGGTTCAACACAGACCCCACC
ATGTTCGCTTATCTGAGGTCCCTGCAGGCTTTCAGTAACCCCTCGGCTGGCATCTCAGTGGATGTGAAAGTC
TTCATCGTGCCTGTGGGAAATCATTCCAACATCCCGTTCAGCCGCGTGAACCACAGCAAGTTCATGGTCACA
GACAAGACAGCCTATGTAGGCACCTCTAACTGGTCAGAAGACTACTTCAGCCACACCGCTGGTGTGGGCCTG
ATTGTCAGCCAGAAGACCCCCAGAGCCCAGCCAGGCGCAACCACCGTGCAGGAGCAGCTGAGGCAACTCTTT
GAACGAGACTGGAGTTCCCACTATGCTATGGACCTAGACAGACAAGTCCCGAGCCAGGACTGTGTCTGGTAG
> Mouse PLD4 protein (503 amino acids)
(SEQ ID NO: 132)
MDKKKEHPEMRIPLQTAVEVSDWPCSTSHDPHSGLGMVLGMLAVLGLSSVTLILFLWQGATSFTSHRMFPEE
VPSWSWETLKGDAEQQNNSCQLILVESIPEDLPFAAGSPTAQPLAQAWLQLLDTARESVHIASYYWSLTGLD
IGVNDSSSRQGEALLQKFQQLLLRNISVVVATHSPTLAKTSTDLQVLAAHGAQIRQVPMKQLTGGVLHSKFW
VVDGRHVYVGSANMDWRSLTQVKELGAIIYNCSNLAQDLEKTFQTYWVLGTPQAVLPKTWPRNFSSHINRFH
PLRGPFDGVPTTAYFSASPPSLCPHGRTRDLDAVLGVMEGARQFIYVSVMEYFPTTRFTHHARYWPVLDNAL
RAAALNKGVHVRLLVSCWFNTDPTMFAYLRSLQAFSNPSAGISVDVKVFIVPVGNHSNIPFSRVNHSKFMVT
DKTAYVGTSNWSEDYFSHTAGVGLIVSQKTPRAQPGATTVQEQLRQLFERDWSSHYAMDLDRQVPSQDCVW
> Human PLD3 cDNA sequence
(SEQ ID NO: 55)
ATGAAGCCTAAACTGATGTACCAGGAGCTGAAGGTGCCTGCAGAGGAGCCCGCCAATGAGCTGCCCATGAAT
GAGATTGAGGCGTGGAAGGCTGCGGAAAAGAAAGCCCGCTGGGTCCTGCTGGTCCTCATTCTGGCGGTTGTG
GGCTTCGGAGCCCTGATGACTCAGCTGTTTCTATGGGAATACGGCGACTTGCATCTCTTTGGGCCCAACCAG
CGCCCAGCCCCCTGCTATGACCCTTGCGAAGCAGTGCTGGTGGAAAGCATTCCTGAGGGCCTGGACTTCCCC
AATGCCTCCACGGGGAACCCTTCCACCAGCCAGGCCTGGCTGGGCCTGCTCGCCGGTGCGCACAGCAGCCTG
GACATCGCCTCCTTCTACTGGACCCTCACCAACAATGACACCCACACGCAGGAGCCCTCTGCCCAGCAGGGT
GAGGAGGTCCTCCGGCAGCTGCAGACCCTGGCACCAAAGGGCGTGAACGTCCGCATCGCTGTGAGCAAGCCC
AGCGGGCCCCAGCCACAGGCGGACCTGCAGGCTCTGCTGCAGAGCGGTGCCCAGGTCCGCATGGTGGACATG
CAGAAGCTGACCCATGGCGTCCTGCATACCAAGTTCTGGGTGGTGGACCAGACCCACTTCTACCTGGGCAGT
GCCAACATGGACTGGCGTTCACTGACCCAGGTCAAGGAGCTGGGCGTGGTCATGTACAACTGCAGCTGCCTG
GCTCGAGACCTGACCAAGATCTTTGAGGCCTACTGGTTCCTGGGCCAGGCAGGCAGCTCCATCCCATCAACT
TGGCCCCGGTTCTATGACACCCGCTACAACCAAGAGACACCAATGGAGATCTGCCTCAATGGAACCCCTGCT
CTGGCCTACCTGGCGAGTGCGCCCCCACCCCTGTGTCCAAGTGGCCGCACTCCAGACCTGAAGGCTCTACTC
AACGTGGTGGACAATGCCCGGAGTTTCATCTACGTCGCTGTCATGAACTACCTGCCCACTCTGGAGTTCTCC
CACCCTCACAGGTTCTGGCCTGCCATTGACGATGGGCTGCGGCGGGCCACCTACGAGCGTGGCGTCAAGGTG
CGCCTGCTCATCAGCTGCTGGGGACACTCGGAGCCATCCATGCGGGCCTTCCTGCTCTCTCTGGCTGCCCTG
CGTGACAACCATACCCACTCTGACATCCAGGTGAAACTCTTTGTGGTCCCCGCGGATGAGGCCCAGGCTCGA
ATCCCATATGCCCGTGTCAACCACAACAAGTACATGGTGACTGAACGCGCCACCTACATCGGAACCTCCAAC
TGGTCTGGCAACTACTTCACGGAGACGGCGGGCACCTCGCTGCTGGTGACGCAGAATGGGAGGGGCGGCCTG
CGGAGCCAGCTGGAGGCCATTTTCCTGAGGGACTGGGACTCCCCTTACAGCCATGACCTTGACACCTCAGCT
GACAGCGTGGGCAACGCCTGCCGCCTGCTCTGA
> Human PLD3 protein (490 amino acids)
(SEQ ID NO: 127)
MKPKLMYQELKVPAEEPANELPMNEIEAWKAAEKKARWVLLVLILAVVGFGALMTQLFLWEYGDLHLFGPNQ
RPAPCYDPCEAVLVESIPEGLDFPNASTGNPSTSQAWLGLLAGAHSSLDIASFYWTLTNNDTHTQEPSAQQG
EEVLRQLQTLAPKGVNVRIAVSKPSGPQPQADLQALLQSGAQVRMVDMQKLTHGVLHTKFWVVDQTHFYLGS
ANMDWRSLTQVKELGVVMYNCSCLARDLTKIFEAYWFLGQAGSSIPSTWPRFYDTRYNQETPMEICLNGTPA
LAYLASAPPPLCPSGRTPDLKALLNVVDNARSFIYVAVMNYLPTLEFSHPHRFWPAIDDGLRRATYERGVKV
RLLISCWGHSEPSMRAFLLSLAALRDNHTHSDIQVKLFVVPADEAQARIPYARVNHNKYMVTERATYIGTSN
WSGNYFTETAGTSLLVTQNGRGGLRSQLEAIFLRDWDSPYSHDLDTSADSVGNACRLL
> Human PLD5 cDNA (1338 base pairs)
(SEQ ID NO: 56)
ATGGGAGAGGATGAGGATGGACTCTCAGAAAAAAATTGCCAAAATAAATGTCGAATTGCCCTGGTGGAAAAT
ATTCCTGAAGGCCTTAACTATTCAGAAAATGCACCATTTCACTTATCACTTTTCCAAGGCTGGATGAATTTA
CTCAACATGGCCAAAAAGTCTGTTGACATAGTGTCTTCCCATTGGGATCTCAACCACACTCATCCATCAGCA
TGTCAGGGTCAACGTCTTTTTGAAAAGTTGCTCCAGCTGACTTCGCAAAATATTGAAATCAAGCTAGTGAGT
GATGTAACAGCTGATTCAAAGGTATTAGAAGCCTTGAAATTAAAGGGAGCCGAGGTGACGTACATGAACATG
ACCGCTTACAACAAGGGCCGGCTGCAGTCCTCCTTCTGGATCGTGGACAAACAGCACGTGTATATCGGCAGT
GCCGGTTTGGACTGGCAATCCCTGGGACAGATGAAAGAACTCGGTGTCATCTTCTACAACTGCAGCTGCCTG
GTCCTAGATTTACAAAGGATATTTGCTCTATATAGTTCATTAAAATTCAAAAGCAGAGTGCCTCAAACCTGG
TCCAAAAGACTCTATGGAGTCTATGACAATGAAAAGAAATTGCAACTTCAGTTGAATGAAACCAAATCTCAA
GCATTTGTATCGAATTCTCCAAAACTCTTTTGCCCTAAAAACAGAAGTTTTGACATAGATGCCATCTACAGT
GTGATAGATGATGCCAAGCAGTATGTGTACATCGCTGTCATGGACTACCTGCCTATCTCCAGCACAAGCACC
AAAAGGACTTACTGGCCAGACTTGGATGCAAAAATAAGAGAAGCATTAGTTTTACGAAGCGTTAGAGTTCGA
CTCCTTTTAAGCTTCTGGAAGGAAACTGATCCCCTTACGTTTAACTTTATTTCATCTCTTAAAGCGATTTGC
ACTGAAATAGCCAACTGCAGTTTGAAAGTTAAATTTTTTGATCTGGAAAGAGAGAATGCTTGTGCTACAAAA
GAACAAAAGAATCACACCTTTCCTAGGTTAAATCGCAACAAGTACATGGTGACAGATGGAGCAGCTTATATT
GGAAATTTTGATTGGGTAGGGAATGATTTCACTCAGAATGCTGGCACGGGCCTTGTTATCAACCAGGCAGAT
GTGAGGAACAACAGAAGCATCATTAAGCAACTTAAAGATGTGTTTGAAAGGGACTGGTATTCACCGTATGCC
AAAACCTTACAGCCAACCAAACAGCCGAACTGCTCAAGCCTGTTCAAACTCAAACCCCTCTCCAACAAAACT
GCCACAGACGACACAGGCGGAAAGGATCCCCGGAACGTATGA
> Human PLD5 protein (445 amino acids)
(SEQ ID NO: 128)
MGEDEDGLSEKNCQNKCRIALVENIPEGLNYSENAPFHLSLFQGWMNLLNMAKKSVDIVSSHWDLNHTHPSA
CQGQRLFEKLLQLTSQNIEIKLVSDVTADSKVLEALKLKGAEVTYMNMTAYNKGRLQSSFWIVDKQHVYIGS
AGLDWQSLGQMKELGVIFYNCSCLVLDLQRIFALYSSLKFKSRVPQTWSKRLYGVYDNEKKLQLQLNETKSQ
AFVSNSPKLFCPKNRSFDIDAIYSVIDDAKQYVYIAVMDYLPISSTSTKRTYWPDLDAKIREALVLRSVRVR
LLLSFWKETDPLTFNFISSLKAICTEIANCSLKVKFFDLERENACATKEQKNHTFPRLNRNKYMVTDGAAYI
GNFDWVGNDFTQNAGTGLVINQADVRNNRSIIKQLKDVFERDWYSPYAKTLQPTKQPNCSSLFKLKPLSNKT
ATDDTGGKDPRNV
> Human PLD4-Ig fusion protein cDNA (2142 bp)
(SEQ ID NO: 125)
ATGGAGTTTCAGACCCAGGTCTTTGTATTCGTGTTGCTCTGGTTGTCTGGTGTTGATGGAgattacaaggat
gacgacgataaaGGATCCcccagagggcccacaatcaagccctgtcctccatgcaaatgcccagcacctaac
ctcttgggtggaccatccgtcttcatcttccctccaaagatcaaggatgtactcatgatctccctgagcccc
atagtcacatgtgtggtggtggatgtgagcgaggatgacccagatgtccagatcagctggtttgtgaacaac
gtggaagtacacacagctcagacacaaacccatagagaggattacaacagtactctccgggtggtcagtgcc
ctccccatccagcaccaggactggatgagtggcaaggagttcaaatgcaaggtcaacaacaaagacctccca
gcgcccatcgagagaaccatctcaaaacccaaagggtcagtaagagctccacaggtatatgtcttgcctcca
ccagaagaagagatgactaagaaacaggtcactctgacctgcatggtcacagacttcatgcctgaagacatt
tacgtggagtggaccaacaacgggaaaacagagctaaactacaagaacactgaaccagtcctggactctgat
ggttcttacttcatgtacagcaagctgagagtggaaaagaagaactgggtggaaagaaatagctactcctgt
tcagtggtccacgagggtctgcacaatcaccacacgactaagagcttctcccggactccgggtaaaCGTCCT
CCCACCTGGGGCCAGGTGCAGCCCAAGGACGTGCCCAGGTCCTGGGAGCATGGCTCCAGCCCAGCTTGGGAG
CCCCTGGAAGCAGAGGCCAGGCAGCAGAGGGACTCCTGCCAGCTTGTCCTTGTGGAAAGCATCCCCCAGGAC
CTGCCATCTGCAGCCGGCAGCCCCTCTGCCCAGCCTCTGGGCCAGGCCTGGCTGCAGCTGCTGGACACTGCC
CAGGAGAGCGTCCACGTGGCTTCATACTACTGGTCCCTCACAGGGCCTGACATCGGGGTCAACGACTCGTCT
TCCCAGCTGGGAGAGGCTCTTCTGCAGAAGCTGCAGCAGCTGCTGGGCAGGAACATTTCCCTGGCTGTGGCC
ACCAGCAGCCCGACACTGGCCAGGACATCCACCGACCTGCAGGTTCTGGCTGCCCGAGGTGCCCATGTACGA
CAGGTGCCCATGGGGCGGCTCACCAGGGGTGTTTTGCACTCCAAATTCTGGGTTGTGGATGGACGGCACATA
TACATGGGCAGTGCCAACATGGACTGGCGGTCTCTGACGCAGGTGAAGGAGCTTGGCGCTGTCATCTATAAC
TGCAGCCACCTGGCCCAAGACCTGGAGAAGACCTTCCAGACCTACTGGGTACTGGGGGTGCCCAAGGCTGTC
CTCCCCAAAACCTGGCCTCAGAACTTCTCATCTCACTTCAACCGTTTCCAGCCCTTCCACGGCCTCTTTGAT
GGGGTGCCCACCACTGCCTACTTCTCAGCGTCGCCACCAGCACTCTGTCCCCAGGGCCGCACCCGGGACCTG
GAGGCGCTGCTGGCGGTGATGGGGAGCGCCCAGGAGTTCATCTATGCCTCCGTGATGGAGTATTTCCCCACC
ACGCGCTTCAGCCACCCCCCGAGGTACTGGCCGGTGCTGGACAACGCGCTGCGGGCGGCAGCCTTCGGCAAG
GGCGTGCGCGTGCGCCTGCTGGTCGGCTGCGGACTCAACACGGACCCCACCATGTTCCCCTACCTGCGGTCC
CTGCAGGCGCTCAGCAACCCCGCGGCCAACGTCTCTGTGGACGTGAAAGTCTTCATCGTGCCGGTGGGGAAC
CATTCCAACATCCCATTCAGCAGGGTGAACCACAGCAAGTTCATGGTCACGGAGAAGGCAGCCTACATAGGC
ACCTCCAACTGGTCGGAGGATTACTTCAGCAGCACGGCGGGGGTGGGCTTGGTGGTCACCCAGAGCCCTGGC
GCGCAGCCCGCGGGGGCCACGGTGCAGGAGCAGCTGCGGCAGCTCTTTGAGCGGGACTGGAGTTCGCGCTAC
GCCGTCGGCCTGGACGGACAGGCTCCGGGCCAGGACTGCGTTTGGCAGGGCTGA
> Human PLD4-Ig fusion protein (713 amino acids)
(SEQ ID NO: 126)
MEFQTQVFVFVLLWLSGVDGDYKDDDDKGSPRGPTIKPCPPCKCPAPNLLGGPSVFIFPPKIKDVLMISLSP
IVTCVVVDVSEDDPDVQISWFVNNVEVHTAQTQTHREDYNSTLRVVSALPIQHQDWMSGKEFKCKVNNKDLP
APIERTISKPKGSVRAPQVYVLPPPEEEMTKKQVTLTCMVTDFMPEDIYVEWTNNGKTELNYKNTEPVLDSD
GSYFMYSKLRVEKKNWVERNSYSCSVVHEGLHNHHTTKSFSRTPGKRPPTWGQVQPKDVPRSWEHGSSPAWE
PLEAEARQQRDSCQLVLVESIPQDLPSAAGSPSAQPLGQAWLQLLDTAQESVHVASYYWSLTGPDIGVNDSS
SQLGEALLQKLQQLLGRNISLAVATSSPTLARTSTDLQVLAARGAHVRQVPMGRLTRGVLHSKFWVVDGRHI
YMGSANMDWRSLTQVKELGAVIYNCSHLAQDLEKTFQTYWVLGVPKAVLPKTWPQNFSSHFNRFQPFHGLFD
GVPTTAYFSASPPALCPQGRTRDLEALLAVMGSAQEFIYASVMEYFPTTRFSHPPRYWPVLDNALRAAAFGK
GVRVRLLVGCGLNTDPTMFPYLRSLQALSNPAANVSVDVKVFIVPVGNHSNIPFSRVNHSKFMVTEKAAYIG
TSNWSEDYFSSTAGVGLVVTQSPGAQPAGATVQEQLRQLFERDWSSRYAVGLDGQAPGQDCVWQG

### Accession Numbers

### NITE ABP-1211

### NITE ABP-1212

### NITE ABP-1213

### NITE ABP-1214

### Sequence Listing Free Text

SEQ ID NO: 45: Forward primer
SEQ ID NO: 46: Reverse primer
SEQ ID NO: 47: Forward primer
SEQ ID NO: 48: Reverse primer
SEQ ID NO: 49: Forward primer
SEQ ID NO: 50: Reverse primer
SEQ ID NO: 51: Forward primer
SEQ ID NO: 52: Reverse primer
SEQ ID NO: 53: Forward primer
SEQ ID NO: 54: Reverse primer
SEQ ID NO: 70: Anchor primer
SEQ ID NO: 70: n is deoxyinosine
SEQ ID NO: 71: AUAP primer

### SEQ ID NO: 72: Primer

### SEQ ID NO: 73: Primer

### SEQ ID NO: 114: Primer

### SEQ ID NO: 115: Primer

### SEQ ID NO: 116: Primer

### SEQ ID NO: 117: Primer

### SEQ ID NO: 118: Primer

### SEQ ID NO: 119: Primer

### [Sequence Listing]

1.-32. (canceled)

33. A method for detecting activated B cells, the method comprising:
(a) contacting cells to be tested with an antibody, or an antibody fragment containing an antigen-binding region thereof, that binds to human phospholipase D4, wherein the antibody or antibody fragment containing the antigen-binding region thereof comprises:
a heavy chain CDR1 set forth in SEQ ID NO: 2, a heavy chain CDR2 set forth in SEQ ID NO: 3, a heavy chain CDR3 set forth in SEQ ID NO: 4, a light chain CDR1 set forth in SEQ ID NO: 5, a light chain CDR2 as set forth in SEQ ID NO: 6, and a light chain CDR3 set forth in SEQ ID NO: 7;
a heavy chain CDR1 set forth in SEQ ID NO: 8, a heavy chain CDR2 set forth in SEQ ID NO: 9, a heavy chain CDR3 set forth in SEQ ID NO: 10, a light chain CDR1 set forth in SEQ ID NO: 11, a light chain CDR2 set forth in SEQ ID NO: 12 and a light chain CDR3 set forth in SEQ ID NO: 13;
a heavy chain CDR1 set forth in SEQ ID NO: 14, a heavy chain CDR2 set forth in SEQ ID NO: 15, a heavy chain CDR3 set forth in SEQ ID NO: 16, a light chain CDR1 set forth in SEQ ID NO: 17, a light chain CDR2 set forth in SEQ ID NO: 18 and a light chain CDR3 set forth in SEQ ID NO: 19;
a heavy chain CDR1 set forth in SEQ ID NO: 20, a heavy chain CDR2 set forth in SEQ ID NO: 21, a heavy chain CDR3 set forth in SEQ ID NO: 22, a light chain CDR1 set forth in SEQ ID NO: 23, a light chain CDR2 set forth in SEQ ID NO: 24 and a light chain CDR3 set forth in SEQ ID NO: 25;
a heavy chain CDR1 set forth in SEQ ID NO: 26, a heavy chain CDR2 set forth in SEQ ID NO: 27, a heavy chain CDR3 set forth in SEQ ID NO: 28, a light chain CDR1 set forth in SEQ ID NO: 29, a light chain CDR2 set forth in SEQ ID NO: 30 and a light chain CDR3 set forth in SEQ ID NO: 31;
a heavy chain CDR1 set forth in SEQ ID NO: 32, a heavy chain CDR2 set forth in SEQ ID NO: 33, a heavy chain CDR3 set forth in SEQ ID NO: 34, a light chain CDR1 set forth in SEQ ID NO: 35, a light chain CDR2 set forth in SEQ ID NO: 36 and a light chain CDR3 set forth in SEQ ID NO: 37; or
a heavy chain CDR1 set forth in SEQ ID NO: 38, a heavy chain CDR2 set forth in SEQ ID NO: 39, a heavy chain CDR3 set forth in SEQ ID NO: 40, a light chain CDR1 set forth in SEQ ID NO: 41, a light chain CDR2 set forth in SEQ ID NO: 42 and a light chain CDR3 set forth in SEQ ID NO: 43; and
(b) detecting binding of the antibody or antibody fragment containing the antigen-binding region thereof to the cells.

34. The method of claim 33, wherein the antibody or antibody fragment containing the antigen-binding region thereof is chimeric or humanized.

35. A method for detecting activated B cells, the method comprising:
(a) contacting cells to be tested with an antibody, or an antibody fragment containing an antigen-binding region thereof, that binds to human phospholipase D4, wherein the antibody is a monoclonal antibody produced by any one of hybridomas mp5B7, mp7B4, mp13D4, or mp13H11 of Deposit Nos. NITE BP-1211, NITE BP-1212, NITE BP-1213, or NITE BP-1214 or a chimeric or humanized version thereof; and
(b) detecting binding of the antibody or antibody fragment containing the antigen-binding region thereof to the cells.


SEQUENCE LISTING (132 sequences)

SEQ ID NO: 1 - protein, 506 aa, Homo sapiens
  PEPTIDE (1)..(506)
MLKPLWKAAVAPTWPCSMPPRRPWDREAGTLQVLGALAVLWLGSVALICLLWQVPRPPTWGQVQPKDVPRSWEHGSSPAWEPLEAEARQQRDSCQLVLVESIPQDLPSAAGSPSAQPLGQAWLQLLDTAQESVHVASYYWSLTGPDIGVNDSSSQLGEALLQKLQQLLGRNISLAVATSSPTLARTSTDLQVLAARGAHVRQVPMGRLTRGVLHSKFWVVDGRHIYMGSANMDWRSLTQVKELGAVIYNCSHLAQDLEKTFQTYWVLGVPKAVLPKTWPQNFSSHFNRFQPFHGLFDGVPTTAYFSASPPALCPQGRTRDLEALLAVMGSAQEFIYASVMEYFPTTRFSHPPRYWPVLDNALRAAAFGKGVRVRLLVGCGLNTDPTMFPYLRSLQALSNPAANVSVDVKVFIVPVGNHSNIPFSRVNHSKFMVTEKAAYIGTSNWSEDYFSSTAGVGLVVTQSPGAQPAGATVQEQLRQLFERDWSSRYAVGLDGQAPGQDCVWQG

SEQ ID NO: 2 - protein, 5 aa, Homo sapiens
SYWMH

SEQ ID NO: 3 - protein, 17 aa, Homo sapiens
DIYPGSDSTNYNEKFKS

SEQ ID NO: 4 - protein, 9 aa, Homo sapiens
GGWLDAMDY

SEQ ID NO: 5 - protein, 11 aa, Homo sapiens
RASQDISNYLN

SEQ ID NO: 6 - protein, 7 aa, Homo sapiens
YTSRLHS

SEQ ID NO: 7 - protein, 8 aa, Homo sapiens
QQGNTLPW

SEQ ID NO: 8 - protein, 5 aa, Homo sapiens
TYWMH

SEQ ID NO: 9 - protein, 17 aa, Homo sapiens
AIYPGNSETSYNQKFKG

SEQ ID NO: 10 - protein, 7 aa, Homo sapiens
GYSDFDY

SEQ ID NO: 11 - protein, 11 aa, Homo sapiens
HASQGIRSNIG

SEQ ID NO: 12 - protein, 7 aa, Homo sapiens
HGTNLED

SEQ ID NO: 13 - protein, 7 aa, Homo sapiens
VQYVQFP

SEQ ID NO: 14 - protein, 5 aa, Homo sapiens
DYNLH

SEQ ID NO: 15 - protein, 17 aa, Homo sapiens
YIYPYNGNTGYNQKFKR

SEQ ID NO: 16 - protein, 14 aa, Homo sapiens
GGIYDDYYDYAIDY

SEQ ID NO: 17 - protein, 11 aa, Homo sapiens
RASENIYSHIA

SEQ ID NO: 18 - protein, 7 aa, Homo sapiens
GATNLAH

SEQ ID NO: 19 - protein, 7 aa, Homo sapiens
QHFWGTP

SEQ ID NO: 20 - protein, 5 aa, Homo sapiens
SYYLY

SEQ ID NO: 21 - protein, 17 aa, Homo sapiens
LINPTNSDTIFNEKFKS

SEQ ID NO: 22 - protein, 11 aa, Homo sapiens
EGGYGYGPFAY

SEQ ID NO: 23 - protein, 16 aa, Homo sapiens
TSSQTLVHSNGNTYLH

SEQ ID NO: 24 - protein, 7 aa, Homo sapiens
KVSNRFS

SEQ ID NO: 25 - protein, 6 aa, Homo sapiens
HSTHVP

SEQ ID NO: 26 - protein, 5 aa, Homo sapiens
SYGMS

SEQ ID NO: 27 - protein, 17 aa, Homo sapiens
TISSGGSYIYYPESVKG

SEQ ID NO: 28 - protein, 12 aa, Homo sapiens
LYGGRRGYGLDY

SEQ ID NO: 29 - protein, 16 aa, Homo sapiens
RSSKSLLHSDGITYLY

SEQ ID NO: 30 - protein, 7 aa, Homo sapiens
QMSNLAS

SEQ ID NO: 31 - protein, 6 aa, Homo sapiens
AQNLEL

SEQ ID NO: 32 - protein, 6 aa, Homo sapiens
SHYYWT

SEQ ID NO: 33 - protein, 16 aa, Homo sapiens
YISYDGSNNYNPSLKN

SEQ ID NO: 34 - protein, 15 aa, Homo sapiens
EGPLYYGNPYWYFDV

SEQ ID NO: 35 - protein, 11 aa, Homo sapiens
RASQDIDNYLN

SEQ ID NO: 36 - protein, 7 aa, Homo sapiens
YTSRLHS

SEQ ID NO: 37 - protein, 7 aa, Homo sapiens
QQFNTLP

SEQ ID NO: 38 - protein, 6 aa, Homo sapiens
SHYYWS

SEQ ID NO: 39 - protein, 16 aa, Homo sapiens
YISYDGSNNYNPSLKN

SEQ ID NO: 40 - protein, 15 aa, Homo sapiens
EGPLYYGNPYWYFDV

SEQ ID NO: 41 - protein, 11 aa, Homo sapiens
RASQDIDNYLN

SEQ ID NO: 42 - protein, 7 aa, Homo sapiens
YTSRLHS

SEQ ID NO: 43 - protein, 7 aa, Homo sapiens
QQFNTLP

SEQ ID NO: 44 - DNA, 1521 bp, Homo sapiens
atgctgaagcctctttggaaagcagcagtggcccccacatggccatgctccatgccgccccgccgcccgtgggacagagaggctggcacgttgcaggtcctgggagcgctggctgtgctgtggctgggctccgtggctcttatctgcctcctgtggcaagtgccccgtcctcccacctggggccaggtgcagcccaaggacgtgcccaggtcctgggagcatggctccagcccagcttgggagcccctggaagcagaggccaggcagcagagggactcctgccagcttgtccttgtggaaagcatcccccaggacctgccatctgcagccggcagcccctctgcccagcctctgggccaggcctggctgcagctgctggacactgcccaggagagcgtccacgtggcttcatactactggtccctcacagggcctgacatcggggtcaacgactcgtcttcccagctgggagaggctcttctgcagaagctgcagcagctgctgggcaggaacatttccctggctgtggccaccagcagcccgacactggccaggacatccaccgacctgcaggttctggctgcccgaggtgcccatgtacgacaggtgcccatggggcggctcaccaggggtgttttgcactccaaattctgggttgtggatggacggcacatatacatgggcagtgccaacatggactggcggtctctgacgcaggtgaaggagcttggcgctgtcatctataactgcagccacctggcccaagacctggagaagaccttccagacctactgggtactgggggtgcccaaggctgtcctccccaaaacctggcctcagaacttctcatctcacttcaaccgtttccagcccttccacggcctctttgatggggtgcccaccactgcctacttctcagcgtcgccaccagcactctgtccccagggccgcacccgggacctggaggcgctgctggcggtgatggggagcgcccaggagttcatctatgcctccgtgatggagtatttccccaccacgcgcttcagccaccccccgaggtactggccggtgctggacaacgcgctgcgggcggcagccttcggcaagggcgtgcgcgtgcgcctgctggtcggctgcggactcaacacggaccccaccatgttcccctacctgcggtccctgcaggcgctcagcaaccccgcggccaacgtctctgtggacgtgaaagtcttcatcgtgccggtggggaaccattccaacatcccattcagcagggtgaaccacagcaagttcatggtcacggagaaggcagcctacataggcacctccaactggtcggaggattacttcagcagcacggcgggggtgggcttggtggtcacccagagccctggcgcgcagcccgcgggggccacggtgcaggagcagctgcggcagctctttgagcgggactggagttcgcgctacgccgtcggcctggacggacaggctccgggccaggactgcgtttggcagggctga

SEQ ID NO: 45 - DNA, 18 bp, Artificial
  forward primer
atggactggcggtctctg

SEQ ID NO: 46 - DNA, 20 bp, Artificial
  reverse primer
tggaaggtcttctccaggtc

SEQ ID NO: 47 - DNA, 19 bp, Artificial
  forward primer
agccacatcgctcagacac

SEQ ID NO: 48 - DNA, 19 bp, Artificial
  reverse primer
gcccaatacgaccaaatcc

SEQ ID NO: 49 - DNA, 18 bp, Artificial
  forward primer
atggactggcggtctctg

SEQ ID NO: 50 - DNA, 20 bp, Artificial
  reverse primer
tggaaggtcttctccaggtc

SEQ ID NO: 51 - DNA, 19 bp, Artificial
  forward primer
agccacatcgctcagacac

SEQ ID NO: 52 - DNA, 19 bp, Artificial
  reverse primer
gcccaatacgaccaaatcc

SEQ ID NO: 53 - DNA, 30 bp, Artificial
  forward primer
tttgaattcgccgccaccatgctgaagcct

SEQ ID NO: 54 - DNA, 34 bp, Artificial
  reverse primer
aaagcggccgctcagccctgccaaacgcagtcct

SEQ ID NO: 55 - DNA, 1473 bp, Homo sapiens
  gene (1)..(1473)
atgaagcctaaactgatgtaccaggagctgaaggtgcctgcagaggagcccgccaatgagctgcccatgaatgagattgaggcgtggaaggctgcggaaaagaaagcccgctgggtcctgctggtcctcattctggcggttgtgggcttcggagccctgatgactcagctgtttctatgggaatacggcgacttgcatctctttgggcccaaccagcgcccagccccctgctatgacccttgcgaagcagtgctggtggaaagcattcctgagggcctggacttccccaatgcctccacggggaacccttccaccagccaggcctggctgggcctgctcgccggtgcgcacagcagcctggacatcgcctccttctactggaccctcaccaacaatgacacccacacgcaggagccctctgcccagcagggtgaggaggtcctccggcagctgcagaccctggcaccaaagggcgtgaacgtccgcatcgctgtgagcaagcccagcgggccccagccacaggcggacctgcaggctctgctgcagagcggtgcccaggtccgcatggtggacatgcagaagctgacccatggcgtcctgcataccaagttctgggtggtggaccagacccacttctacctgggcagtgccaacatggactggcgttcactgacccaggtcaaggagctgggcgtggtcatgtacaactgcagctgcctggctcgagacctgaccaagatctttgaggcctactggttcctgggccaggcaggcagctccatcccatcaacttggccccggttctatgacacccgctacaaccaagagacaccaatggagatctgcctcaatggaacccctgctctggcctacctggcgagtgcgcccccacccctgtgtccaagtggccgcactccagacctgaaggctctactcaacgtggtggacaatgcccggagtttcatctacgtcgctgtcatgaactacctgcccactctggagttctcccaccctcacaggttctggcctgccattgacgatgggctgcggcgggccacctacgagcgtggcgtcaaggtgcgcctgctcatcagctgctggggacactcggagccatccatgcgggccttcctgctctctctggctgccctgcgtgacaaccatacccactctgacatccaggtgaaactctttgtggtccccgcggatgaggcccaggctcgaatcccatatgcccgtgtcaaccacaacaagtacatggtgactgaacgcgccacctacatcggaacctccaactggtctggcaactacttcacggagacggcgggcacctcgctgctggtgacgcagaatgggaggggcggcctgcggagccagctggaggccattttcctgagggactgggactccccttacagccatgaccttgacacctcagctgacagcgtgggcaacgcctgccgcctgctctga

SEQ ID NO: 56 - DNA, 1338 bp, Homo sapiens
  gene (1)..(1338)
atgggagaggatgaggatggactctcagaaaaaaattgccaaaataaatgtcgaattgccctggtggaaaatattcctgaaggccttaactattcagaaaatgcaccatttcacttatcacttttccaaggctggatgaatttactcaacatggccaaaaagtctgttgacatagtgtcttcccattgggatctcaaccacactcatccatcagcatgtcagggtcaacgtctttttgaaaagttgctccagctgacttcgcaaaatattgaaatcaagctagtgagtgatgtaacagctgattcaaaggtattagaagccttgaaattaaagggagccgaggtgacgtacatgaacatgaccgcttacaacaagggccggctgcagtcctccttctggatcgtggacaaacagcacgtgtatatcggcagtgccggtttggactggcaatccctgggacagatgaaagaactcggtgtcatcttctacaactgcagctgcctggtcctagatttacaaaggatatttgctctatatagttcattaaaattcaaaagcagagtgcctcaaacctggtccaaaagactctatggagtctatgacaatgaaaagaaattgcaacttcagttgaatgaaaccaaatctcaagcatttgtatcgaattctccaaaactcttttgccctaaaaacagaagttttgacatagatgccatctacagtgtgatagatgatgccaagcagtatgtgtacatcgctgtcatggactacctgcctatctccagcacaagcaccaaaaggacttactggccagacttggatgcaaaaataagagaagcattagttttacgaagcgttagagttcgactccttttaagcttctggaaggaaactgatccccttacgtttaactttatttcatctcttaaagcgatttgcactgaaatagccaactgcagtttgaaagttaaattttttgatctggaaagagagaatgcttgtgctacaaaagaacaaaagaatcacacctttcctaggttaaatcgcaacaagtacatggtgacagatggagcagcttatattggaaattttgattgggtagggaatgatttcactcagaatgctggcacgggccttgttatcaaccaggcagatgtgaggaacaacagaagcatcattaagcaacttaaagatgtgtttgaaagggactggtattcaccgtatgccaaaaccttacagccaaccaaacagccgaactgctcaagcctgttcaaactcaaacccctctccaacaaaactgccacagacgacacaggcggaaaggatccccggaacgtatga

SEQ ID NO: 57 - DNA, 39 bp, Artificial
  forward primer
tttaagcttgccgccaccatgaagcctaaactgatgtac

SEQ ID NO: 58 - DNA, 57 bp, Artificial
  reverse primer
tttgaattctcacttatcgtcgtcatccttgtaatcgagcaggcggcaggcgttgcc

SEQ ID NO: 59 - DNA, 39 bp, Artificial
  forward primer
tttaagcttgccgccaccatgggagaggatgaggatgga

SEQ ID NO: 60 - DNA, 57 bp, Artificial
  reverse primer
tttgaattctcacttatcgtcgtcatccttgtaatctacgttccggggatcctttcc

SEQ ID NO: 61 - DNA, 26 bp, Artificial
  forward primer
agatgctgaagcctcttcggagagcg

SEQ ID NO: 62 - DNA, 25 bp, Artificial
  reverse primer
tcagccctgccaaacgcagtcctgg

SEQ ID NO: 63 - DNA, 1521 bp, Macaca fascicularis
  gene (1)..(1521)
atgctgaagcctcttcggagagcggcagtgacccccatgtggccgtgctccatgctgccccgccgcctgtgggacagagaggctggcacgttgcaggtcctgggagtgctggctatgctgtggctgggctccatggctcttacctacctcctgtggcaagtgcgccgtcctcccacctggggccaggtgcagcccaaggacgtgcccaggtcctgggggcatggttccagcccagctctggagcccctggaagcggaggtcaggaagcagagggactcctgccagcttgtccttgtggaaagcatcccccaggacctgccatttgcagccggcagcctctccgcccagcctctgggccaggcctggctgcagctgctggacactgcccaggagagcgtccacgtggcttcatactactggtccctcacagggcccgacattggggtcaacgactcatcttcccagctgggagaggcccttctgcagaagctgcagcagctgctgggcaggaacatttccttggctgtggccaccagcagtccaacactggccaggaagtccaccgacctgcaggtcctggctgcccgaggtgcccaggtacgacgggtgcccatggggcggctcaccaggggcgttttgcactccaaattctgggttgtggatggacggcacatatacatgggcagtgccaacatggactggcggtccctgacgcaggtgaaggagcttggcgctgtcatctataactgcagccacctggcccaagacctggagaagaccttccagacctactgggtgctgggggtgcccaaggctgtcctccccaaaacctggcctcagaacttctcatctcacatcaaccgtttccagcccttccagggcctctttgatggggtgcccaccactgcctacttctcagcatcgccacccgcactctgtccccagggccgcacccctgacctggaggcgctgttggcggtgatggggagcgcccaggagttcatctatgcctccgtgatggagtatttccctaccacgcgcttcagccacccccgcaggtactggccggtgctggacaacgcgctgcgggcggcagccttcagcaagggtgtgcgcgtgcgcctgctggtcagctgcggactcaacacggaccccaccatgttcccctatctgcggtccctgcaggcgctcagcaaccccgcggccaacgtctctgtggacgtgaaagtcttcatcgtgccggtggggaatcattccaacatcccgttcagcagggtgaaccacagcaagttcatggtcacggagaaggcagcctacataggcacctccaactggtcggaggattacttcagcagcacgacgggggtgggcctggtggtcacccagagccccggcgcgcagcccgcgggggccacggtacaggagcagctgcggcagctctttgagcgggactggagttcgcgctacgccgtcggcctggacggacaggctccgggccaggactgcgtttggcagggctga

SEQ ID NO: 64 - DNA, 36 bp, Artificial
  primer
ccaggagagtgggagaggctcttctcagtatggtgg

SEQ ID NO: 65 - DNA, 32 bp, Artificial
  primer
ggctcagggaaatagcccttgaccaggcatcc

SEQ ID NO: 66 - DNA, 24 bp, Artificial
  primer
tccagagttccaggtcactgtcac

SEQ ID NO: 67 - DNA, 24 bp, Artificial
  primer
aggggccagtggatagacagatgg

SEQ ID NO: 68 - DNA, 24 bp, Artificial
  primer
tccagagttccaagtcacagtcac

SEQ ID NO: 69 - DNA, 24 bp, Artificial
  primer
aggggccagtggatagactgatgg

SEQ ID NO: 70 - DNA, 36 bp, Artificial
  anchor primer
  misc_feature (24)..(25): n is deoxyisosine.
  misc_feature (29)..(30): n is deoxyisosine.
  misc_feature (34)..(35): n is deoxyisosine.
ggccacgcgtcgactagtacgggnngggnngggnng

SEQ ID NO: 71 - DNA, 20 bp, Artificial
  AUAP primer
ggccacgcgtcgactagtac

SEQ ID NO: 72 - DNA, 31 bp, Artificial
  primer
cactacttcctgttgaagctcttgacgatgg

SEQ ID NO: 73 - DNA, 23 bp, Artificial
  primer
gtgagtggcctcacaggtatagc

SEQ ID NO: 74 - DNA, 504 bp, Mus musculus
atgagatcacagttctctatacagttactgagcacacagaacctcaccttgggatggagctgtatcatcctcttcttggtagcaacagctacaggtgtccactcccaggtccaactgcagcagcctggggctgaactggtgaagcctgggacttcagtgaaaatgtcctgcaaggcttctggctacaccttcaccagctactggatgcactgggtgaagcagaggccgggacaaggccttgagtggattggagatatttatcctggtagtgatagtactaactacaatgagaagttcaagagcaaggccacactgactgtagacacatcctccagcacagcctacatgcaactcagcagcctgacatctgaggactctgcggtctattactgtgcaagaggagggtggttggatgctatggactactggggtcaaggaacctcagtcaccgtctcctcagccaaaacaacacccccatcagtctatccactggcccctaagggc

SEQ ID NO: 75 - protein, 168 aa, Mus musculus
MRSQFSIQLLSTQNLTLGWSCIILFLVATATGVHSQVQLQQPGAELVKPGTSVKMSCKASGYTFTSYWMHWVKQRPGQGLEWIGDIYPGSDSTNYNEKFKSKATLTVDTSSSTAYMQLSSLTSEDSAVYYCARGGWLDAMDYWGQGTSVTVSSAKTTPPSVYPLAPKG

SEQ ID NO: 76 - DNA, 437 bp, Mus musculus
atggaatgtaactggatacttccttttattctgtcggtaatttcaggggtctcctcagaggttcagctccagcagtctgggactgtgctgtcaaggcctggggcttccgtgacgatgtcctgcaaggcttctggcgacagctttaccacctactggatgcactgggtaaaacagaggcctggacagggtctagaatggattggtgctatctatcctggaaatagtgaaactagctacaaccagaagttcaagggcaaggccaaactgactgcagtcacatccgccagcactgcctatatggagttcactagcctgacaaatgaggactctgcggtctattactgtacggggggttattccgactttgactactggggccaaggcaccactctcacagtctcctcagccaaaacgacacccccatctgtctatccact

SEQ ID NO: 77 - protein, 145 aa, Mus musculus
MECNWILPFILSVISGVSSEVQLQQSGTVLSRPGASVTMSCKASGDSFTTYWMHWVKQRPGQGLEWIGAIYPGNSETSYNQKFKGKAKLTAVTSASTAYMEFTSLTNEDSAVYYCTGGYSDFDYWGQGTTLTVSSAKTTPPSVYP

SEQ ID NO: 78 - DNA, 475 bp, Mus musculus
atgggatggagctggatctttctcttcctcctgtcaggaactgcaggcgtccactctgaggtccagcttcagcagtcaggacctgaactggtgaaacctggggcctcagtgaagatatcctgcaaggcttctggatacacattcactgactacaacttgcactgggtgaagcagagccatggaaagagccttgagtggattggatatatttatccttacaatggtaatactggctacaaccagaagttcaagaggaaggccacattgactgtagacaattcctccggcacagtctacatggagctccgcagcctgacatctgaggactctgcagtctattactgtgcaagaggagggatctatgatgattactacgactatgctatcgactattggggtcaaggaacctcagtcaccgtctcctcagccaaaacaacacccccatcagtctatccactggcccctaagggcgaat

SEQ ID NO: 79 - protein, 158 aa, Mus musculus
MGWSWIFLFLLSGTAGVHSEVQLQQSGPELVKPGASVKISCKASGYTFTDYNLHWVKQSHGKSLEWIGYIYPYNGNTGYNQKFKRKATLTVDNSSGTVYMELRSLTSEDSAVYYCARGGIYDDYYDYAIDYWGQGTSVTVSSAKTTPPSVYPLAPKGE

SEQ ID NO: 80 - DNA, 470 bp, Mus musculus
atgggatggagctggatctttctcttcctcctgtcaggaactgcaggcgtccactctgaggtccagcttcagcagtcaggacctgaactggtgaaacctggggcctcagtgaagatatcctgcaaggcttctggatacacattcactgactacaacttgcactgggtgaagcagagccatggaaagagccttgagtggattggatatatttatccttacaatggtaatactggctacaaccagaagttcaagaggaaggccacattgactgtagacaattcctccggcacagtctacatggagctccgcagcctgacatctgaggactctgcagtctattactgtgcaagaggagggatctatgatgattactacgactatgctatcgactattggggtcaaggaacctcagtcaccgtctcctcagccaaaacaacacccccatcagtctatccactggcccctaaggg

SEQ ID NO: 81 - protein, 156 aa, Mus musculus
MGWSWIFLFLLSGTAGVHSEVQLQQSGPELVKPGASVKISCKASGYTFTDYNLHWVKQSHGKSLEWIGYIYPYNGNTGYNQKFKRKATLTVDNSSGTVYMELRSLTSEDSAVYYCARGGIYDDYYDYAIDYWGQGTSVTVSSAKTTPPSVYPLAPK

SEQ ID NO: 82 - DNA, 462 bp, Mus musculus
atgggatggagctatatcatcctctttttggtagcaacagcaacaggggtccactcccaggtccaactgcagcagtcgggggctgaactggtgaagcctggggcttcagtgaagttgtcctgcaaggcttctggctacaccttcaccagctactatttgtactgggtgaggcagaggcctggacaaggccttgagtggattggactgattaatcctaccaatagtgatactatcttcaatgagaagttcaagagcaaggccacactgactgtagacaaatcctccagcacagcatacatgcaactcagcagcctgacatctgaggactctgcggtctattactgtacacgagaggggggatatggttacggcccgtttgcttactggggccaagggactctggtcactgtctctgcagccaaaacaacacccccatcagtctatccactggcccctaagggc

SEQ ID NO: 83 - protein, 154 aa, Mus musculus
MGWSYIILFLVATATGVHSQVQLQQSGAELVKPGASVKLSCKASGYTFTSYYLYWVRQRPGQGLEWIGLINPTNSDTIFNEKFKSKATLTVDKSSSTAYMQLSSLTSEDSAVYYCTREGGYGYGPFAYWGQGTLVTVSAAKTTPPSVYPLAPKG

SEQ ID NO: 84 - DNA, 450 bp, Mus musculus
atgaacttcgggctcagcttgattttccttgccctcattttaaaaggtgtccagtgtgaggtgcagctggtggagtctgggggagacttagtgaggcctggagggtccctgaaactctcctgtgcagcctctggattcagtttcagtagctatggcatgtcttggtttcgccagactccagacaagaggctggagtgggtcgcaaccattagtagtggtggtagttacatctactatccagaaagtgtgaaggggcgattcaccatctccagagacaatgccaggaacatcctgtacctgcaaatgagcagtctgaagtctgaggacacagccatgtattattgtgtaagactctacggtggtaggagaggctatggtttggactactggggtcaaggaacctcagtcaccgtctcctcagccaaaacaacagccccatcggtctatcca

SEQ ID NO: 85 - protein, 150 aa, Mus musculus
MNFGLSLIFLALILKGVQCEVQLVESGGDLVRPGGSLKLSCAASGFSFSSYGMSWFRQTPDKRLEWVATISSGGSYIYYPESVKGRFTISRDNARNILYLQMSSLKSEDTAMYYCVRLYGGRRGYGLDYWGQGTSVTVSSAKTTAPSVYP

SEQ ID NO: 86 - DNA, 450 bp, Mus musculus
atgaacttcgggctcagcttgattttccttgccctcattttaaaaggtgtccagtgtgaggtgcagctggtggagtctgggggagacttagtgaggcctggagggtccctgaaactctcctgtgcagcctctggattcagtttcagtagctatggcatgtcttggtttcgccagactccagacaagaggctggagtgggtcgcaaccattagtagtggtggtagttacatctactatccagaaagtgtgaaggggcgattcaccatctccagagacaatgccaggaacatcctgtacctgcaaatgagcagtctgaagtctgaggacacagccatgtattattgtgtaagactctacggtggtaggagaggctatggtttggactactggggtcaaggaacctcagtcaccgtctcctcagccaaaacaacacccccatcagtctatcca

SEQ ID NO: 87 - protein, 150 aa, Mus musculus
MNFGLSLIFLALILKGVQCEVQLVESGGDLVRPGGSLKLSCAASGFSFSSYGMSWFRQTPDKRLEWVATISSGGSYIYYPESVKGRFTISRDNARNILYLQMSSLKSEDTAMYYCVRLYGGRRGYGLDYWGQGTSVTVSSAKTTPPSVYP

SEQ ID NO: 88 - DNA, 472 bp, Mus musculus
atgaaagtgttgagtctgttgtacctgttgacagccattcctggtatcctgtctgatgtacagcttcaggagtcaggacctggcctcgtgaaaccttctcaatctctgtctctcacctgctctgtcactggctactccatcaccagtcattattactggacctggatccggcagtttccaggaaacaaactggaatggatgggctacataagctacgacggtagcaataactacaacccatctctcaaaaatcgaatctccatcactcgtgacacatctaagaaccagtttttcctgaagttgaattctgtgactactgaggacacagctacatataactgtgcaagagagggcccgctctactatggtaacccctactggtatttcgatgtctggggcgcagggaccacggtcaccgtctcctcagccaaaacaacacccccatcagtctatccactggcccctaagggcg

SEQ ID NO: 89 - protein, 157 aa, Mus musculus
MKVLSLLYLLTAIPGILSDVQLQESGPGLVKPSQSLSLTCSVTGYSITSHYYWTWIRQFPGNKLEWMGYISYDGSNNYNPSLKNRISITRDTSKNQFFLKLNSVTTEDTATYNCAREGPLYYGNPYWYFDVWGAGTTVTVSSAKTTPPSVYPLAPKG

SEQ ID NO: 90 - DNA, 471 bp, Mus musculus
atgaaagtgttgagtctgttgtacctgttgacagccattcctggtatcctgtctgatgtacagcttcaggagtcaggacctggcctcgtgaaaccttctcagtctctgtctctcacctgctctgtcactggctactccatctccagtcattattactggagttggatccggcagtttccaggaaacagactggaatggatgggctacataagctacgacggtagcaataactacaacccatctctcaaaaatcgaatctccatcactcgtgacacatctaagaaccagtttttcctgaagttgaattctgtgactactgaggacacagctacatataactgtgcaagagagggcccgctctactatggtaacccctactggtatttcgatgtctggggcgcagggaccacggtcaccgtctcctcagccaaaacaacacccccatcagtctatccactggcccctaagggc

SEQ ID NO: 91 - protein, 157 aa, Mus musculus
MKVLSLLYLLTAIPGILSDVQLQESGPGLVKPSQSLSLTCSVTGYSISSHYYWSWIRQFPGNRLEWMGYISYDGSNNYNPSLKNRISITRDTSKNQFFLKLNSVTTEDTATYNCAREGPLYYGNPYWYFDVWGAGTTVTVSSAKTTPPSVYPLAPKG

SEQ ID NO: 92 - DNA, 470 bp, Mus musculus
atgaaagtgttgagtctgttgtacctgttgacagccattcctggtatcctgtctgatgtacagcttcaggagtcaggacctggcctcgtgaaaccttctcagtctctgtctctcacctgctctgtcactggctactccatctccagtcattattactggagttggatccggcagtttccaggaaacagactggaatggatgggctacataagctacgacggtagcaataactacaacccatctctcaaaaatcgaatctccatcactcgtgacacatctaagaaccagtttttcctgaagttgaattctgtgactactgaggacacagctacatataactgtgcaagagagggcccgctctactatggtaacccctactggtatttcgatgtctggggcgcagggaccacggtcaccgtctcctcagccaaaacgacacccccatctgtctatccactggcccctaaggg

SEQ ID NO: 93 - protein, 156 aa, Mus musculus
MKVLSLLYLLTAIPGILSDVQLQESGPGLVKPSQSLSLTCSVTGYSISSHYYWSWIRQFPGNRLEWMGYISYDGSNNYNPSLKNRISITRDTSKNQFFLKLNSVTTEDTATYNCAREGPLYYGNPYWYFDVWGAGTTVTVSSAKTTPPSVYPLAPK

SEQ ID NO: 94 - DNA, 421 bp, Mus musculus
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaacgggctgatgctgcaccaactgtatccatcaagggcgaat

SEQ ID NO: 95 - protein, 140 aa, Mus musculus
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRADAAPTVSIKGE

SEQ ID NO: 96 - DNA, 459 bp, Mus musculus
atgatggtccttgctcagtttcttgcattcttgttgctttggtttccaggtgcaggatgtgacatcctgatgacccaatctccatcctccatgtctgtatctctgggagacacagtcagcatcacttgccatgcaagtcagggcattagaagtaatatagggtggttgcagcagaaaccagggaaatcatttaagggcctgatctttcatggaaccaacttggaagatggagttccatcaaggttcagtggcagaggatctggagcagattattctctcaccatcaacagcctggaatctgaagattttgcagactattactgtgtacagtatgttcagtttcctccaacgttcggctcggggacaaagttggaaataagacgggctgatgctgcaccaactgtatccatcttcccaccatccagtgagcagttaacatctggaggtgcctcagtcgtg

SEQ ID NO: 97 - protein, 153 aa, Mus musculus
MMVLAQFLAFLLLWFPGAGCDILMTQSPSSMSVSLGDTVSITCHASQGIRSNIGWLQQKPGKSFKGLIFHGTNLEDGVPSRFSGRGSGADYSLTINSLESEDFADYYCVQYVQFPPTFGSGTKLEIRRADAAPTVSIFPPSSEQLTSGGASVV

SEQ ID NO: 98 - DNA, 467 bp, Mus musculus
atgagtgtgcccactcaggtcctggggttgctgctgctgtggcttacagatgccagatgtgacatccagatgactcagtctccagcctccctatctgtatctgtgggagaaactgtcgccatcacatgtcgagcaagtgagaatatttacagtcatatagcatggtatcagcagaaagagggaaaatctcctcagcgcctggtctatggtgcaacaaacttagcacatggtgtgccatcaaggttcagtggcagtggatcaggcacacagtattccctcaagatcaacagccttcagtctgaagattttgggagttattactgtcaacatttttggggtactccgtggacgttcggtggaggcaccaagctggaaatcaaacgggctgatgctgcaccaactgtatccatcttcccaccatccagtgagcagttaacatctggaggtgcctcagtcgtgtgcttctt

SEQ ID NO: 99 - protein, 155 aa, Mus musculus
MSVPTQVLGLLLLWLTDARCDIQMTQSPASLSVSVGETVAITCRASENIYSHIAWYQQKEGKSPQRLVYGATNLAHGVPSRFSGSGSGTQYSLKINSLQSEDFGSYYCQHFWGTPWTFGGGTKLEIKRADAAPTVSIFPPSSEQLTSGGASVVCF

SEQ ID NO: 100 - DNA, 454 bp, Mus musculus
atgagtgtgcccactcaggtcctggggttgctgctgctgtggcttacagatgccagatgtgacatccagatgactcagtctccagcctccctatctgtatctgtgggagaaactgtcgccatcacatgtcgagcaagtgagaatatttacagtcatatagcatggtatcagcagaaagagggaaaatctcctcagcgcctggtctatggtgcaacaaacttagcacatggtgtgccatcaaggttcagtggcagtggatcaggcacacagtattccctcaagatcaacagccttcagtctgaagattttgggagttattactgtcaacatttttggggtactccgtggacgttcggtggaggcaccaagctggaaatcaaacgggctgatgctgcaccaactgtatccatcttcccaccatccagtgagcagttaacatctggaggtgcctcag

SEQ ID NO: 101 - protein, 151 aa, Mus musculus
MSVPTQVLGLLLLWLTDARCDIQMTQSPASLSVSVGETVAITCRASENIYSHIAWYQQKEGKSPQRLVYGATNLAHGVPSRFSGSGSGTQYSLKINSLQSEDFGSYYCQHFWGTPWTFGGGTKLEIKRADAAPTVSIFPPSSEQLTSGGAS

SEQ ID NO: 102 - DNA, 457 bp, Mus musculus
atgaagttgcctgttaggctgttggtgctgatgttctggattcctgcttccagcagtgatgttgtgatgacccaaactccactctccctgcctgtcagtcttggagatcaagcctccatctcttgcacatctagtcagacccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcacagtacacatgttccattcacgttcggctcggggacaaagttggaaataaaacgggctgatgctgcaccaactgtatccatcttcccaccatccagtgagcagttaacatctggag

SEQ ID NO: 103 - protein, 152 aa, Mus musculus
MKLPVRLLVLMFWIPASSSDVVMTQTPLSLPVSLGDQASISCTSSQTLVHSNGNTYLHWYLQKPGQSPKLLIYKVSNRFSGVPDRFSGSGSGTDFTLKISRVEAEDLGVYFCSHSTHVPFTFGSGTKLEIKRADAAPTVSIFPPSSEQLTSG

SEQ ID NO: 104 - DNA, 423 bp, Mus musculus
atgaggttctctgctcagcttctggggctgcttgtgctctggatccctggatccactgcggaaattgtgatgacgcaggctgcattctccaatccagtcactcttggaacatcagcttccatctcctgcaggtctagtaagagtctcctacatagtgatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaactttacacgttcggaggggggaccaagctggaaataaaacgggctgatgctgcaccaactgtatccatc

SEQ ID NO: 105 - protein, 141 aa, Mus musculus
MRFSAQLLGLLVLWIPGSTAEIVMTQAAFSNPVTLGTSASISCRSSKSLLHSDGITYLYWYLQKPGQSPQLLIYQMSNLASGVPDRFSSSGSGTDFTLRISRVEAEDVGVYYCAQNLELYTFGGGTKLEIKRADAAPTVSI

SEQ ID NO: 106 - DNA, 423 bp, Mus musculus; identical to SEQ ID NO: 104

SEQ ID NO: 107 - protein, 141 aa, Mus musculus; identical to SEQ ID NO: 105

SEQ ID NO: 108 - DNA, 404 bp, Mus musculus
atgatgtcctctgctcagttccttggtctcctgttgctctgttttcaaggtaccagatgtgatatccagatgacacagactacatcctccctgtctgcctctctgggggacagagtcaccatcagttgcagggcaagtcaggacattgacaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatgttgccacttacttttgccagcagtttaatacgcttcctcggacgttcggtggaggcaccaaactggaaatcaaacgggctgatgctgcaccaactgt

SEQ ID NO: 109 - protein, 134 aa, Mus musculus
MMSSAQFLGLLLLCFQGTRCDIQMTQTTSSLSASLGDRVTISCRASQDIDNYLNWYQQKPDGTVKLLIYYTSRLHSGVPSRFSGSGSGTDYSLTISNLEQEDVATYFCQQFNTLPRTFGGGTKLEIKRADAAPT

SEQ ID NO: 110 - DNA, 414 bp, Mus musculus
atgatgtcctctgctcagttccttggtctcctgttgctctgttttcaaggtaccagatgtgatatccagatgacacagactacatcctccctgtctgcctctctggggggcagcgtcaccatcagttgcagggcaagtcaggacattgacaattatttaaactggtatcagcaaaaaccagatggaactgttaaactcctgatctactacacatcaagattacactcaggagtcccatcaaggttcagtggcagtgggtctggaacagattattctctcaccattagcaacctggaacaagaagatattgccacttacttttgccaacagtttaatacgcttcctcggacgttcggtggaggcaccaagctggaaatcaaacgggctgatgctgcaccaactgtatccatcttc

SEQ ID NO: 111 - protein, 138 aa, Mus musculus
MMSSAQFLGLLLLCFQGTRCDIQMTQTTSSLSASLGGSVTISCRASQDIDNYLNWYQQKPDGTVKLLIYYTSRLHSGVPSRFSGSGSGTDYSLTISNLEQEDIATYFCQQFNTLPRTFGGGTKLEIKRADAAPTVSIF

SEQ ID NO: 112 - DNA, 465 bp, Mus musculus
atgatgtcctctgctcagttccttggtctcctgttgctctgttttcaaggtaccagatgtgatatccagatgacacagactacatcctccctgtctgcctctctggggggcagcgtcaccatcagttgcagggcaagtcaggacattgacaattatttaaactggtatcagcaaaaaccagatggaactgttaaactcctgatctactacacatcaagattacactcaggagtcccatcaaggttcagtggcagtgggtctggaacagattattctctcaccattagcaacctggaacaagaagatattgccacttacttttgccaacagtttaatacgcttcctcggacgttcggtggaggcaccaagctggaaatcaaacgggctgatgctgcaccaactgtatccatcttcccaccatccagtgagcagttaacatctggaggtgcctcagtcgtgtgcttc

SEQ ID NO: 113 - protein, 155 aa, Mus musculus
MMSSAQFLGLLLLCFQGTRCDIQMTQTTSSLSASLGGSVTISCRASQDIDNYLNWYQQKPDGTVKLLIYYTSRLHSGVPSRFSGSGSGTDYSLTISNLEQEDIATYFCQQFNTLPRTFGGGTKLEIKRADAAPTVSIFPPSSEQLTSGGASVVCF

SEQ ID NO: 114 - DNA, 93 bp, Artificial
  primer
accaagcttgccgccaccatgaaagtgttgagtctgttgtacctgttgacagccattcctggtatcctgtctcaggtccaactgcagcagcct

SEQ ID NO: 115 - DNA, 42 bp, Artificial
  primer
cgatgggcccttggtgctagctgaggagacggtgactgaggt

SEQ ID NO: 116 - DNA, 39 bp, Artificial
  primer
accaagcttgccgccaccatgatgtcctctgctcagttc

SEQ ID NO: 117 - DNA, 33 bp, Artificial
  primer
agccacagttcgtttgatttccagcttggtgcc

SEQ ID NO: 118 - DNA, 33 bp, Artificial
  primer
ctggaaatcaaacgaactgtggctgcaccatct

SEQ ID NO: 119 - DNA, 30 bp, Artificial
  primer
aaagaattcctagcactctcccctgttgaa

SEQ ID NO: 120 - DNA, 1401 bp, Chimaera sp.
atgaaagtgttgagtctgttgtacctgttgacagccattcctggtatcctgtctcaggtccaactgcagcagcctggggctgaactggtgaagcctgggacttcagtgaaaatgtcctgcaaggcttctggctacaccttcaccagctactggatgcactgggtgaagcagaggccgggacaaggccttgagtggattggagatatttatcctggtagtgatagtactaactacaatgagaagttcaagagcaaggccacactgactgtagacacatcctccagcacagcctacatgcaactcagcagcctgacatctgaggactctgcggtctattactgtgcaagaggagggtggttggatgctatggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 121 - protein, 466 aa, Chimaera sp.
MKVLSLLYLLTAIPGILSQVQLQQPGAELVKPGTSVKMSCKASGYTFTSYWMHWVKQRPGQGLEWIGDIYPGSDSTNYNEKFKSKATLTVDTSSSTAYMQLSSLTSEDSAVYYCARGGWLDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - DNA, 705 bp, Chimaera sp.
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctag

SEQ ID NO: 123 - protein, 234 aa, Chimaera sp.
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - DNA, 1521 bp, Macaca mulatta
  gene (1)..(1521)
atgctgaagcctcttcggagagcggcagtgacccccatgtggccgtgctccatgctgccccgccgcctgtgggacagagaggctggcacgttgcaggtcctgggagtgctggctatgctgtggctgggctccatggctcttacctacctcctgtggcaagtgcgctgtcctcccacctggggccaggtgcagcccagggacgtgcccaggtcctgggggcatggttccagcctagctctggagcccctggaagcggaggtcaggaagcagagggactcctgccagcttgtccttgtggaaagcatcccccaggacctgccatttgcagccggcagcctctccgcccagcctctgggccaggcctggctgcagctgctggacactgcccaggagagcgtccacgtggcttcatactactggtccctcacagggcccgacattggggtcaacgactcatcttcccagctgggagaggcccttctgcagaagctgcagcagctgctgggcaggaacatttccttggctgtggccaccagcagtccaacactggccaggaagtccaccgacctgcaggtcctggctgcccgaggtgcccaggtacgacgggtgcccatggggcggctcaccaggggcgttttgcactccaaattctgggttgtggatggacggcacatatacatgggcagtgccaacatggactggcggtccctgacgcaggtgaaggagcttggcgctgtcatctataactgcagccacctggcccaagacctggagaagaccttccagacctactgggtgctgggggtgcccaaggctgtcctccccaaaacctggcctcagaacttctcatctcacatcaaccgtttccagcccttccagggcctctttgatggggtgcccaccactgcctacttctcagcatcgccacccgcactctgtccccagggccgcacccctgacctggaggcgctgttggcggtgatggggagcgcccaggagttcatctatgcctccgtgatggagtatttccctaccacgcgcttcagccacccccgcaggtactggccggtgctggacaacgcgctgcgggcggcagccttcagcaagggtgtgcgcgtgcgcctgctggtcagctgcggactcaacacggaccccaccatgttcccctatctgcggtccctgcaggcgctcagcaaccccgcggccaacgtctctgtggacgtgaaagtcttcatcgtgccggtggggaatcattccaacatcccgttcagcagggtgaaccacagcaagttcatggtcacggagaaggcagcctacataggcacctccaactggtcggaggattacttcagcagcacgacgggggtgggcctggtggtcacccagagccccggcgcgcagcccgcgggggccacggtacaggagcagctgcggcagctctttgagcgggactggagttcgcgctacgccgtcggcctggacggacaggctccgggccaggactgcgtttggcagggctga

SEQ ID NO: 125 - DNA, 2142 bp, Homo sapiens
atggagtttcagacccaggtctttgtattcgtgttgctctggttgtctggtgttgatggagattacaaggatgacgacgataaaggatcccccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaacgtcctcccacctggggccaggtgcagcccaaggacgtgcccaggtcctgggagcatggctccagcccagcttgggagcccctggaagcagaggccaggcagcagagggactcctgccagcttgtccttgtggaaagcatcccccaggacctgccatctgcagccggcagcccctctgcccagcctctgggccaggcctggctgcagctgctggacactgcccaggagagcgtccacgtggcttcatactactggtccctcacagggcctgacatcggggtcaacgactcgtcttcccagctgggagaggctcttctgcagaagctgcagcagctgctgggcaggaacatttccctggctgtggccaccagcagcccgacactggccaggacatccaccgacctgcaggttctggctgcccgaggtgcccatgtacgacaggtgcccatggggcggctcaccaggggtgttttgcactccaaattctgggttgtggatggacggcacatatacatgggcagtgccaacatggactggcggtctctgacgcaggtgaaggagcttggcgctgtcatctataactgcagccacctggcccaagacctggagaagaccttccagacctactgggtactgggggtgcccaaggctgtcctccccaaaacctggcctcagaacttctcatctcacttcaaccgtttccagcccttccacggcctctttgatggggtgcccaccactgcctacttctcagcgtcgccaccagcactctgtccccagggccgcacccgggacctggaggcgctgctggcggtgatggggagcgcccaggagttcatctatgcctccgtgatggagtatttccccaccacgcgcttcagccaccccccgaggtactggccggtgctggacaacgcgctgcgggcggcagccttcggcaagggcgtgcgcgtgcgcctgctggtcggctgcggactcaacacggaccccaccatgttcccctacctgcggtccctgcaggcgctcagcaaccccgcggccaacgtctctgtggacgtgaaagtcttcatcgtgccggtggggaaccattccaacatcccattcagcagggtgaaccacagcaagttcatggtcacggagaaggcagcctacataggcacctccaactggtcggaggattacttcagcagcacggcgggggtgggcttggtggtcacccagagccctggcgcgcagcccgcgggggccacggtgcaggagcagctgcggcagctctttgagcgggactggagttcgcgctacgccgtcggcctggacggacaggctccgggccaggactgcgtttggcagggctga

SEQ ID NO: 126 - protein, 713 aa, Homo sapiens
MEFQTQVFVFVLLWLSGVDGDYKDDDDKGSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKRPPTWGQVQPKDVPRSWEHGSSPAWEPLEAEARQQRDSCQLVLVESIPQDLPSAAGSPSAQPLGQAWLQLLDTAQESVHVASYYWSLTGPDIGVNDSSSQLGEALLQKLQQLLGRNISLAVATSSPTLARTSTDLQVLAARGAHVRQVPMGRLTRGVLHSKFWVVDGRHIYMGSANMDWRSLTQVKELGAVIYNCSHLAQDLEKTFQTYWVLGVPKAVLPKTWPQNFSSHFNRFQPFHGLFDGVPTTAYFSASPPALCPQGRTRDLEALLAVMGSAQEFIYASVMEYFPTTRFSHPPRYWPVLDNALRAAAFGKGVRVRLLVGCGLNTDPTMFPYLRSLQALSNPAANVSVDVKVFIVPVGNHSNIPFSRVNHSKFMVTEKAAYIGTSNWSEDYFSSTAGVGLVVTQSPGAQPAGATVQEQLRQLFERDWSSRYAVGLDGQAPGQDCVWQG

SEQ ID NO: 127 - protein, 490 aa, Homo sapiens
MKPKLMYQELKVPAEEPANELPMNEIEAWKAAEKKARWVLLVLILAVVGFGALMTQLFLWEYGDLHLFGPNQRPAPCYDPCEAVLVESIPEGLDFPNASTGNPSTSQAWLGLLAGAHSSLDIASFYWTLTNNDTHTQEPSAQQGEEVLRQLQTLAPKGVNVRIAVSKPSGPQPQADLQALLQSGAQVRMVDMQKLTHGVLHTKFWVVDQTHFYLGSANMDWRSLTQVKELGVVMYNCSCLARDLTKIFEAYWFLGQAGSSIPSTWPRFYDTRYNQETPMEICLNGTPALAYLASAPPPLCPSGRTPDLKALLNVVDNARSFIYVAVMNYLPTLEFSHPHRFWPAIDDGLRRATYERGVKVRLLISCWGHSEPSMRAFLLSLAALRDNHTHSDIQVKLFVVPADEAQARIPYARVNHNKYMVTERATYIGTSNWSGNYFTETAGTSLLVTQNGRGGLRSQLEAIFLRDWDSPYSHDLDTSADSVGNACRLL

SEQ ID NO: 128 - protein, 445 aa, Homo sapiens
MGEDEDGLSEKNCQNKCRIALVENIPEGLNYSENAPFHLSLFQGWMNLLNMAKKSVDIVSSHWDLNHTHPSACQGQRLFEKLLQLTSQNIEIKLVSDVTADSKVLEALKLKGAEVTYMNMTAYNKGRLQSSFWIVDKQHVYIGSAGLDWQSLGQMKELGVIFYNCSCLVLDLQRIFALYSSLKFKSRVPQTWSKRLYGVYDNEKKLQLQLNETKSQAFVSNSPKLFCPKNRSFDIDAIYSVIDDAKQYVYIAVMDYLPISSTSTKRTYWPDLDAKIREALVLRSVRVRLLLSFWKETDPLTFNFISSLKAICTEIANCSLKVKFFDLERENACATKEQKNHTFPRLNRNKYMVTDGAAYIGNFDWVGNDFTQNAGTGLVINQADVRNNRSIIKQLKDVFERDWYSPYAKTLQPTKQPNCSSLFKLKPLSNKTATDDTGGKDPRNV

SEQ ID NO: 129 - protein, 506 aa, Macaca fascicularis
MLKPLRRAAVTPMWPCSMLPRRLWDREAGTLQVLGVLAMLWLGSMALTYLLWQVRRPPTWGQVQPKDVPRSWGHGSSPALEPLEAEVRKQRDSCQLVLVESIPQDLPFAAGSLSAQPLGQAWLQLLDTAQESVHVASYYWSLTGPDIGVNDSSSQLGEALLQKLQQLLGRNISLAVATSSPTLARKSTDLQVLAARGAQVRRVPMGRLTRGVLHSKFWVVDGRHIYMGSANMDWRSLTQVKELGAVIYNCSHLAQDLEKTFQTYWVLGVPKAVLPKTWPQNFSSHINRFQPFQGLFDGVPTTAYFSASPPALCPQGRTPDLEALLAVMGSAQEFIYASVMEYFPTTRFSHPRRYWPVLDNALRAAAFSKGVRVRLLVSCGLNTDPTMFPYLRSLQALSNPAANVSVDVKVFIVPVGNHSNIPFSRVNHSKFMVTEKAAYIGTSNWSEDYFSSTTGVGLVVTQSPGAQPAGATVQEQLRQLFERDWSSRYAVGLDGQAPGQDCVWQG

SEQ ID NO: 130 - protein, 506 aa, Macaca mulatta
MLKPLRRAAVTPMWPCSMLPRRLWDREAGTLQVLGVLAMLWLGSMALTYLLWQVRCPPTWGQVQPRDVPRSWGHGSSLALEPLEAEVRKQRDSCQLVLVESIPQDLPFAAGSLSAQPLGQAWLQLLDTAQESVHVASYYWSLTGPDIGVNDSSSQLGEALLQKLQQLLGRNISLAVATSSPTLARKSTDLQVLAARGAQVRRVPMGRLTRGVLHSKFWVVDGRHIYMGSANMDWRSLTQVKELGAVIYNCSHLAQDLEKTFQTYWVLGVPKAVLPKTWPQNFSSHINRFQPFQGLFDGVPTTAYFSASPPALCPQGRTPDLEALLAVMGSAQEFIYASVMEYFPTTRFSHPRRYWPVLDNALRAAAFSKGVRVRLLVSCGLNTDPTMFPYLRSLQALSNPAANVSVDVKVFIVPVGNHSNIPFSRVNHSKFMVTEKAAYIGTSNWSEDYFSSTTGVGLVVTQSPGAQPAGATVQEQLRQLFERDWSSRYAVGLDGQAPGQDCVWQG

SEQ ID NO: 131 - DNA, 1512 bp, Mus musculus
atggacaagaagaaagagcacccagagatgcggataccactccagacagcagtggaggtctctgattggccctgctccacatctcatgatccacatagcggacttggcatggtactggggatgctagctgtactgggactcagctctgtgactctcatcttgttcctgtggcaaggggccacttctttcaccagtcatcggatgttccctgaggaagtgccctcctggtcctgggagaccctgaaaggagacgctgagcagcagaataactcctgtcagctcatccttgtggaaagcatccccgaggacttgccatttgcagctggcagccccactgcccagcccctggcccaggcttggctgcagcttcttgacactgctcgggagagcgtccacattgcctcgtactactggtccctcactggactggacattggagtcaatgactcgtcttctcggcagggagaggcccttctacagaagttccaacagcttcttctcaggaacatctctgtggtggtggccacccacagcccaacattggccaagacatccactgacctccaggtcttggctgcccatggtgcccagatacgacaagtgcccatgaaacagcttactgggggtgttctacactccaaattctgggttgtggatgggcgacacgtctacgtgggcagcgccaacatggactggcggtccctgactcaggtgaaggaacttggtgcaatcatctacaactgcagcaacctggctcaagaccttgagaaaacattccagacctactgggtgctagggactccccaagctgttctccctaaaacctggcctcggaacttctcatcccacatcaaccgcttccatcccttgcggggtccctttgatggggttcccaccacggcctatttctcggcctcccctccctccctctgcccgcatggccggacccgggatctggacgcagtgttgggagtgatggagggtgctcgccagttcatctatgtctcggtgatggagtatttccctaccacgcgcttcacccaccatgccaggtactggcccgtgctggacaatgcgctacgggcagcggccctcaataagggtgtgcatgtgcgcttactggtcagctgctggttcaacacagaccccaccatgttcgcttatctgaggtccctgcaggctttcagtaacccctcggctggcatctcagtggatgtgaaagtcttcatcgtgcctgtgggaaatcattccaacatcccgttcagccgcgtgaaccacagcaagttcatggtcacagacaagacagcctatgtaggcacctctaactggtcagaagactacttcagccacaccgctggtgtgggcctgattgtcagccagaagacccccagagcccagccaggcgcaaccaccgtgcaggagcagctgaggcaactctttgaacgagactggagttcccactatgctatggacctagacagacaagtcccgagccaggactgtgtctggtag

SEQ ID NO: 132 - protein, 503 aa, Mus musculus
MDKKKEHPEMRIPLQTAVEVSDWPCSTSHDPHSGLGMVLGMLAVLGLSSVTLILFLWQGATSFTSHRMFPEEVPSWSWETLKGDAEQQNNSCQLILVESIPEDLPFAAGSPTAQPLAQAWLQLLDTARESVHIASYYWSLTGLDIGVNDSSSRQGEALLQKFQQLLLRNISVVVATHSPTLAKTSTDLQVLAAHGAQIRQVPMKQLTGGVLHSKFWVVDGRHVYVGSANMDWRSLTQVKELGAIIYNCSNLAQDLEKTFQTYWVLGTPQAVLPKTWPRNFSSHINRFHPLRGPFDGVPTTAYFSASPPSLCPHGRTRDLDAVLGVMEGARQFIYVSVMEYFPTTRFTHHARYWPVLDNALRAAALNKGVHVRLLVSCWFNTDPTMFAYLRSLQAFSNPSAGISVDVKVFIVPVGNHSNIPFSRVNHSKFMVTDKTAYVGTSNWSEDYFSHTAGVGLIVSQKTPRAQPGATTVQEQLRQLFERDWSSHYAMDLDRQVPSQDCVW